(19) United States
(12) Patent Application Publication
(10) Pub. No.: US 2006/0025338 A1
(43) Pub. Date: Feb. 2, 2006

(54) Compositions and methods for treatment of lymphatic and venous vessel arterialization

(75) Inventors: Kari Alitalo, Helsinki (FI); Tatiana Petrova, Helsinki (FI); Terhi Karpanen, Helsinki (FI); Camilla Norrmen, Helsinki (FI)

Correspondence Address: MARSHALL, GERSTEIN & BORUN LLP, 233 S. WACKER DRIVE, SUITE 6300, SEARS TOWER, CHICAGO, IL, 60606, US

(73) Assignees: LUDWIG INSTITUTE FOR CANCER RESEARCH, New York, NY (US); LICENTIA, LTD., Helsinki (FI)

(21) Appl. No.: 11/076,427

(22) Filed: Mar. 8, 2005

Related U.S. Application Data
(60) Provisional application No. 60/551,581, filed on Mar. 8, 2004.

(51) Int. Cl. A61K 38/17; A61K 48/00

(52) U.S. Cl. 514/12; 514/44

(57) ABSTRACT

The present invention is directed to methods and compositions that may be used in disrupting the association of smooth muscle cells with lymphatic endothelial cells and in correcting the valvular dysfunction in veins and lymphatic vessels. Such compositions are useful for therapeutic and prophylactic treatment of impaired lymphatic and venous function, particularly for the treatment of lymphedema distichiasis or chronic venous insufficiency.

The present application claims the benefit of priority of U.S. Patent Application Ser. No. 60/551,581 was filed on Mar. 8, 2004. The entire text of the priority application is incorporated herein by reference in its entirety.

### FIELD OF THE INVENTION

The present invention is generally directed to methods and compositions for the treatment of lymphatic vessel and venous arterialization, including diseases characterized by the same, e.g., lymphedema and chronic venous insufficiency. The invention also is directed to materials and methods for improving lymphatic and venous valves.

### BACKGROUND OF THE INVENTION

A healthy lymphatic system continuously drains lymphatic fluid, consisting of a mixture of lymph, water, proteins and other matter, away from various interstitial areas of the body through a complex network of lymphatic vessels and back into the arterio-venous circulatory system that carries the blood.

Lymph fluid is pumped through the lymphatic system and away from various body areas by both the action of adjacent muscle tissue and the contraction of the larger lymphatic vessels. Foreign matter is filtered out of the lymph fluid as the fluid passes through bundles of lymph nodes during its progress through the lymphatic system. The lymph nodes also monitor the contents of the lymph fluid to determine if any appropriate immune reactions should be initiated by the host's immune system. The lymphatic vessels form a set of coordinated structures including the initial lymphatic sinuses which drain into the lymphatic capillaries and subsequently to the collecting lymphatics which drain into the lymphatic trunks and the thoracic duct which ultimately drains into the blood system after the filtration.

“Lymphedema” describes deficiencies or dysfunctions of the lymphatic system that limit the flow of lymph fluid from a body area. Any sustained accumulation of proteins delivered to the body tissue by the blood capillaries, and not removed by the lymphatic system, will cause an accumulation of high protein fluid in the interstitial areas of the body tissues resulting in lymphedema.

Lymphedema is a disabling and disfiguring condition characterized by swelling of the extremities as a result of lymphatic vessel hypoplasia or obstruction. Patients with lymphedema suffer from recurrent local infections, physical impairment and social anxiety, and may be at increased risk for developing cancers such as lymphangiosarcoma. Hereditary lymphedema may occur as an isolated condition, examples of which include Milroy disease (OMIM 153100) and lymphedema praecox (OMIM 153200), or as a component of a complex syndrome. Several types of hereditary lymphedema have been described so far.

Lymphedema arises after injuries and a variety of surgeries, such as mastectomy, that result in removal of or damage to lymph nodes and vessels. The discovery of a lymphatic growth factor receptor, Flt4 receptor tyrosine kinase (VEGFR-3) and its growth factor ligands VEGF-C and VEGF-D has provided therapeutic tools to promote re-growth of lymphatic vessels in these contents. See U.S. Pat. No. 5,776,755 (VEGFR-3); International Patent Publication Nos. WO 97/05250 and WO 98/33917 (VEGF-C); and WO 98/07832 (VEGF-D).

Mutations in the vascular endothelial growth factor-C (VEGF-C) and VEGF-D receptor VEGFR-3 have been implicated in a subset of primary congenital lymphedemas (Milroy's disease; OMIM 153100; Irrthurm et al. Am J Hum Genet 67, 295-301, 2000; Karkkainen et al., Nat. Genet. 25, 153-159, 2000). Impaired signaling via VEGFR-3, which is important for lymphatic endothelial cell survival, proliferation and migration, results in hypoplasia of the cutaneous lymphatic network and leads to the symptoms of the disease. Treatment with VEGF-C or -D has been described as a therapy for patients with VEGFR-3 hereditary lymphedema. See International Patent Publication No. WO 00/58511.

In the case of lymphedema-distichiasis (LD, OMIM 153400) mutations have been described in the forkhead transcription factor FOXC2 gene (Fang et al., Am. J. Hum. Gen. 67, 1382-1388, 2000; Finegold et al., Hum. Mol. Gen. 10, 1185-1189, 2001; Bell et al., Hum. Gen. 108, 546-551, 2001). In addition to lymphedema, LD patients frequently suffer from chronic venous insufficiency (CVI) and region of chromosome 16 including FOXC2 gene is linked to the development of varicose veins (Brice, G., J Med Genet 39, 478-83, 2002). CVI can lead to chronic, potentially life-threatening infections of the lower extremities. CVI is frequently accompanied by pain, especially after ambulating, and it causes lipodermatosclerosis to the skin of the lower extremities, which lead to eventual skin ulceration. The mechanism underlying the pathology of LD is not well understood. Although LD is hereditary, the average age of onset is about 13 years, leaving a potential window for preventative treatment, if such treatments become available. Targeted inactivation of Foxc2 results in aortic arch, skeletal, genitourinary and cardiac malformations, and leads to embryonic and perinatal lethality. In addition, a similar range of defects occurs in mice double heterozygous for Foxc2 and Foxc1 deletions. (Iida, K. et al., Development 124, 4627-38, 1997; Winnier et al., Genes Dev. 11, 926-940, 1997; Winnier et al., Dev. Biol. 213, 418-431, 1999). The latter encodes a transcription factor with a DNA binding domain almost identical to that of FOXC2. Unlike in congenital lymphedema, lymphatic vessel density is normal or even hyperplastic in LD (Johnson et al., Arch Dermatol 135, 347-8, 1999; Brice, G. et al., J Med Genet 39, 478-83, 2002). A need exists for improved diagnosis of LD and for materials and methods of therapeutic and prophylactic LD treatment.

Pericytes (PCs) are solitary vascular smooth muscle cells that are associated with the finest diameter vessels, and the cells include e.g., mesangial cells in kidney glomeruli and the perisinusoidal fat storing cells in the liver. Vascular smooth muscle cells (SMCs) form concentric layers around larger vessels. In the mature vasculature, SMCs provide structural support to the large vessels, and are important in the regulation of the blood pressure and flow due to their contractility.

Studies from PDGF-B/PDGFRβ-deficient mice, as well as tissue co-culture experiments, have demonstrated that PCs associated with blood capillaries may be necessary for the control of endothelial cell differentiation and proliferation, as well as for the regulation of blood capillary shape and diameter (reviewed in Gerhardt & Betsholtz, Cell Tissue Res., 314, 15-23, 2003). PCs have recently been shown to associate with the angiogenic sprouts of blood vessels (Gerhardt et al., J. Cell Biol. 161, 1163-77, 2003).

While the interactions of SMCs/PCs and blood endothelial cells are the subject of intense research efforts, practically nothing is known about the mechanisms underlying the lack of SMCs in lymphatic capillaries. An impaired PC/SMC recruitment to blood vessels has been reported upon targeted mutagenesis of several genes, such as those encoding PDGF-B/PDGFR-β, Tie-2 and its ligand Ang-1, activin like kinase Alk-1, endoglin, SMAD5, tissue factor and G-protein coupled receptor Edg-1 (Lindahl et al., Science 277, 242-5, 1997; Hellstrom et al., Development 126, 3047-55, 1999; Carmeliet et al., Nature 383, 73-5, 1996; Suri et al., Cell 87, 1161-1169 (1997; Li et al., Science 284, 1534-7, 1999; Yang et al., Development 126, 1571-80, 1999; Liu, Y. et al., J Clin Invest 106, 951-61, 2000; Oh et al. Proc Natl Acad Sci USA 97, 2626-31, 2000)). The impaired recruitment of SMCs to the collecting lymphatic vessels in Ang-2 deficient mice is the only published example of abnormal SMC—lymphatic vessel interaction. (Gale et al., Dev Cell 3, 411-23, 2002). Unlike blood vascular endothelial cells, lymphatic vascular endothelial cells do not produce many basal lamina components and they express distinct sets of cell adhesion molecules, which may account for their differential interaction with PC/SMCs (Petrova et al., EMBO J. 21, 4593-9, 2002; Hirakawa et al., Am J Pathol 162, 575-86 (2003).

### SUMMARY OF THE INVENTION

The present invention provides materials (molecules, compositions, kits, unit doses, etc.) and methods for therapeutic or prophylactic treatment of impaired lymphatic and venous function that can result in conditions where lymphatic vessels or veins become arterialized, a phenomenon that was observed when studying Foxc2 mutant mice and human subjects with LD.

One aspect of the invention is a therapeutic or prophylactic method of improving lymphatic function comprising: administering to a mammalian subject a composition comprising an inhibitor of arterialization of lymphatic vessels, wherein the subject is selected from: subjects with impaired lymphatic function due to arterialization of lymphatic vessels, and subjects with a genetic risk for developing said impaired function. Preferred subjects are human subjects. The description provided herein provides detailed guidance for selecting subjects for therapeutic or prophylactic treatment.

In addition to lymphedema, LD patients frequently suffer from chronic venous insufficiency (CVI, also known as postphlebitic syndrome and postthrombotic syndrome). One apparent cause or frequent characteristic of CVI is the congenital absence of venous valves. Additionally, a region of chromosome 16 that includes the FOXC2 gene has been linked to the development of varicose veins (Brice, G., J Med Genet 39, 478-83, 2002, Ng et al., J Med Genet.: 235-239, 2005). Without being limited to any particular theory of the invention, therapy and prophylaxis described herein for improving lymphatic function in subjects in need thereof also are contemplated as therapy or phrophylaxis for CVI and possibly related conditions of varicose veins or hemorrhoids. Likewise, screening materials and methods of the invention that are useful for diagnosing LD are also useful for diagnosing CVI, varicose veins, or hemorrhoids. For brevity, many aspects of the invention are described below with particular reference to predicting, diagnosing, preventing, or treating lymphedema, LD, or lymphatic function. It should be understood that all such aspects are also intended for predicting, diagnosing, preventing, or treating CVI, varicose veins, or hemorrhoids, especially in patients that suffer from these conditions and also suffer from LD or carry a Foxc2 mutation placing them at risk for LD.

Thus, for example, another aspect of the invention is a therapeutic or prophylactic method of improving venous function comprising: administering to a mammalian subject a composition comprising an inhibitor of arterialization of venous vessels, wherein the subject is selected from: subjects with impaired lymphatic function due to arterialization of lymphatic vessels, subjects with impaired venous function due to arterialization of venous vessels, and subjects with a genetic risk for developing either of said impaired functions.

In related aspects, the invention includes use of an inhibitor of arterialization to treat or prevent impaired lymphatic or venous function; and use of an inhibitor of arterialization in the manufacture of a medicament to treat or prevent impaired lymphatic or venous function.

For the purposes of this invention “arterialization of lymphatic vessels” refers to the phenomenon, details of which are described herein, where lymphatic vessels acquire structural characteristics of blood vessels, including but not limited to: elevated investment of pericytes and/or smooth muscle cells with the lymphatic capillaries (compared to the low levels observed in normal healthy lymphatic vessels); expression of a smooth muscle cell marker in lymphatic capillaries from the subject; expression of (elevated levels of) growth factors such as PDGF-B by lymphatic endothelia, which normal do not express significant amounts of PDGF-B; expression in lymphatic vessels of blood vessel basal lamina components (e.g., proteins) such as collagen IV; tortuous and distended dermal lymphatic capillaries; smooth muscle cells associated with lymphatic vessels; pericytes associated with lymphatic vessels; lymphatic hyperplasia; and/or expression of other genetic markers or affiliation of other cells normally associated with blood vessels. The phenomenon is described herein in at least two contexts. First, laboratory animals carrying mutations in the forkhead trasnscription factor Foxc2 gene (and possibly analogous mutations in the foxc1 gene) are described, and the phenomenon is evaluated pathologically as well as at the molecular level. Arterialization of lymphatic vessels also is described herein in human subjects with lymphedema distichiasis (LD). Even if the phenomenon arises from other genetic causes, infection, or other events, therapies described herein are contemplated. In both the animals and the humans where this phenomenon is observed, impaired lymphatic function also is observed.

A variety of criteria are available for identifying impaired lymphatic function, including edema, swelling, abnormal lymphatic drainage, increased number of lymph nodes, lymph flowback, a lymphatic capillary network that comprises smooth muscle cells associated with lymphatic cells.

The fact that LD symptoms in human subjects do not arise until, on average, age thirteen, a window of time exists during childhood during which individuals at risk for heredity lymphedema can receive prophylactic (preventative) treatment. In a preferred situation, individual subjects are screened for a genetic mutation (e.g., a Foxc2 mutation) that correlates with arterialization of lymphatic vessels or with LD, and subjects that are identified as carrying a mutation or other genetic marker for the disease receive prophylactic treatment to inhibit or prevent edema, arterialization of lymphatic vessels, and/or other indicia (e.g., molecular indicia) of onset of the disease. Subjects may be screened any time after conception, as an embryo or fetus, as an infant, or as a child.

Genetic and other screening described herein also are useful and informative for subjects that are already exhibiting impaired lymphatic function, such as a lymphedema phenotype, in order to determine whether an arterialization of lymphatic endothelia is occurring, or whether other causes are at work, such as a VEGFR-3 mutation.

By “screening for an increased risk” is meant a determination of whether a genetic variation exists in the human subject that correlates with a greater likelihood of developing LD or other arterialization of the lymphatics than exists for the human population as a whole, or for a relevant racial or ethnic human sub-population to which the individual belongs. Both positive and negative determinations (i.e., determinations that a genetic predisposition marker is present or is absent) are intended to fall within the scope of screening according to the invention. As described herein, the presence of a mutation altering the sequence or expression of at least one Foxc2 allele in nucleic acid from a subject is correlated with an increased risk of developing such a lymphatic disorder. The term “Foxc2 mutation genotype” is intended to describe individuals with a Foxc2 mutation in at least one Foxc2 allele that alters FOXC2 expression or function to result in the arterialization or LD phenotype described herein. Criteria for assessing genetic risk for LD include: one or more of the following: (A) at least one genetic relative with hereditary lymphedema; (B) a Foxc2 mutation genotype; (C) a Foxc1 mutation genotype; (D) distichiasis; (E) smooth muscle cells covering lymphatic capillaries; (F) hyperplastic cutaneous lymphatic vessel density; (G) PDGFR-beta-expressing cells associated with lymphatic vessels; (H) PDGF-B overexpression in lymphatic vessels (I) smooth muscle cell association with lymphatic capillaries; (J) pericyte association with lymphatic capillaries; (K) absent or dysfunctional lymphatic valves and (L) expression of blood vessel basal lamina components in lymphatic vessels.

Similarly, the invention includes screening subjects for increased risk of developing CVI based on the same criteria.

A subject that has at least one genetic relative with hereditary lymphedema and has a Foxc2 mutation genotype is diagnosed as high risk for developing LD and chronic venous insufficiency and is an especially good candidate for prophylactic treatment, and/or monitoring for early therapeutic intervention.

A variety of criteria are available for adjusting and optimizing the dose and dosing regimen. For example, for therapeutic administration, the inhibitor of arterialization is preferably administered in an amount effective to reduce edema, reduce pain from edema, increase limb function, increase FOXC2 expression in lymphatic endothelia, reduce PDGF-B expression in lymphatic endothelia, reduce SMC association with lymphatic capillaries, reduce pericyte association with lymphatic capillaries, or combinations of these benchmarks. Reduction of lymphatic is another criteria, and can be judged by lymphoscintigraphy.

For treatment of CVI, varicose veins, or hemorrhoids, the therapeutic is administered in amounts effective to reduce edema, reduce varicose veins or hemorrhoids, reduce pain, pressure, burning, ache or other reported symptoms, and/or improve healing of ulcers.

Any substance that inhibits one or more of the lymphatic “arterialization” phenomena described herein is an inhibitor of arterialization for use according to the invention.

One preferred class of inhibitors is inhibitors of PDGFR-beta activity. Such inhibitors include any substance that directly acts on PDGFR-beta to inhibit its activity; substances that act on PDGFR-beta ligands to inhibit PDGFR-beta activation; substances that act on downstream signaling pathways; and substances that inhibit any of these components of the PDGFR-beta signaling cascade by inhibiting expression of any of the components.

Preferred groups of inhibitors of PDGFR-beta activity include (A) inhibitors of PDGFR-beta expression; (B) inhibitors of PDGF-B stimulation of PDGFR-beta; (C) inhibitors of PDGF-B expression; (D) inhibitors of PDGFR-beta signaling; and (E) combinations thereof.

Preferred inhibitors of PDGFR-beta expression include (A) an antisense molecule directed to PDGFR-beta; (B) an interfering RNA (RNAi) directed to PDGFR-beta; (C) an aptamer that binds PDGFR-beta RNA; (D) a ribozyme directed to PDGFR-beta; and (E) combinations thereof.

Preferred inhibitors of PDGF-B stimulation of PDGFR-beta include: (A) an antibody substance that binds to the extracellular domain of PDGFR-beta and inhibits PDGF binding; (B) an antibody substance that binds to PDGF-B and inhibits the PDGF-B from binding or activating PDGFR-beta; (C) a polypeptide comprising a soluble fragment of PDGFR-beta, wherein the polypeptide and fragment bind PDGF-B; (D) a fragment of PDGF-B that binds and fails to stimulate PDGFR-beta; (E) a polypeptide comprising a soluble fragment of PDGFR-alpha, wherein the polypeptide and fragment bind PDGF-B; and (F) combinations thereof.

Preferred inhibitors of PDGF-B expression include (A) an antisense molecule directed to PDGF-B; (B) interfering RNA (RNAi) directed to PDGF-B; (C) an aptamer that binds PDGF-B RNA; (D) a ribozyme directed to PDGF-B; and (E) combinations thereof.

A first step in PDGFR-beta signaling is PDGFR-beta phosphorylation, and a preferred class of inhibitors is tyrosine kinase inhibitors, such as imatinib mesylate and other molecules described herein or elsewhere.

Any route of administration can be suitable for therapeutics described herein. In some variations, oral, intravenous, intraarterial, and other systemic administrations are used. In some variations, local delivery to an edematous limb or other portion of the body, such as administered subcutaneously at a site of edema, is contemplated.

In addition to administration of inhibitors of arterialization, the subjects described herein are expected to benefit from co-therapy with agents that cause growth of new lymphatic vessels. In preferred embodiments, the inhibitor prevents the new vessels from impairment via arterialization. Preferred compositions for stimulating lymphatic vessel growth include growth factor products such as vascular endothelial growth factor C (VEGF-C) protein products, vascular endothelial growth factor D (VEGF-D) protein products, VEGF-C gene therapy products, and VEGF-D gene therapy protein products, as these terms are used in International Patent Publication No. WO 00/58511, “SCREENING AND THERAPY FOR LYMPHATIC DISORDERS INVOLVING THE FLT4 RECEPTOR TYROSINE KINASE (VEGFR-3),” incorporated herein by reference.

Likewise, CVI co-therapy is contemplated. Such co-therapy includes leg elevation, compression stockings, unna boots, and surgery.

Another aspect of the invention is ex vivo gene therapy to accomplish the therapy described herein. For example, the invention includes a therapeutic or prophylactic method of treating arterialization of lymphatic vessels in a mammalian subject, comprising: providing isolated lymphatic endothelial cells or lymphatic endothelial progenitor cells; transforming or transfecting the cells ex vivo with a polynucleotide comprising a nucleotide sequence that encodes an inhibitor of PDGF expression; and administering the transformed or transfected cells to the mammalian subject. The re-introducted cells, which optionally are expanded ex vivo, grow new lymphatic vessels that show less arterialization and therefore less impairment than the existing vessels in the subject. Re-introduction at a site of edema is specifically contemplated.

Analagous therapy for CVI, targeting vascular endothelial cells or endothelial progenitor cells, is contemplated.

Another aspect of the invention is therapy to reduce the impairment caused by arterialization of lymphatic vessels. For example, therapy to palliate the harmful effects of smooth muscle cell on lymphatic vessels is contemplated. In one variation, the invention is a therapeutic or prophylactic method of improving lymphatic function comprising: administering to a mammalian subject a composition comprising a smooth muscle relaxant; wherein the subject is identified as having arterialization of lymphatic vessels; and wherein the smooth muscle relaxant is administered in an amount effective to improve lymphatic function in the subject. Local administration at a site of edema in the subject is specifically contemplated.

Combination therapy with any two or more agents describe herein also is contemplated as an aspect of the invention. Similarly, every combination of agents described herein, packaged together as a new kit, or formulated together as a single composition, is considered an aspect of the invention. Compositions for use according to the invention preferably include the active agent formulated with a pharmaceutically acceptable carrier.

In another embodiment, active agents are delivered directly to the lymphatics using known lymphatic markers, such as liposomes with antibodies or ligands or other binding partners for the lymphatic endothelial cell markers described herein.

The invention also includes kits which comprise therapeutic compounds or compositions of the invention packaged in a manner which facilitates their use to practice methods of the invention. In a simplest embodiment, such a kit includes a compound or composition described herein as useful for practice of the invention packaged in a container such as a sealed bottle or vessel, with a label affixed to the container or included in the package that describes use of the compound or composition to practice the method of the invention. Preferably, the compound or composition is packaged in a unit dosage form. In another embodiment, a kit of the invention includes both a composition for therapy packaged together with a physical device useful for implementing methods of the invention, such as a dermal patch for delivery, or a syringe.

Another aspect of the invention is methods of screening to identify novel therapeutics. For example, the invention includes a method of screening for an agent to improve lymphatic function in mammalian subjects having arterialization of lymphatic vessels, comprising steps of: contacting arterialized lymphatic tissue with a test agent, wherein the arterialized lymphatic tissue comprises lymphatic endothelial cells associated with pericytes or smooth muscle cells; determining if the test agent causes dissociation of lymphatic endothelial cells from pericytes or smooth muscle cells, wherein a test agent that causes the dissociation is selected as an agent to improve lymphatic function. In one variation, the lymphatic tissue is obtained from an organism with a Foxc2 mutation. In another variation, the method further comprises a step of contacting blood vessels with the test agent, and selecting a test agent that preferentially causes smooth muscle dissociation form lymphatic vessels compared to dissociation of smooth muscle cells from blood vessels.

Analagous screening methods are contemplated using venous tissue from a human with CVI or animal model for CVI.

Another aspect of the invention is a vector comprising a polynucleotide comprising a nucleic acid sequence that encodes a polypeptide with FoxC2 transcription factor activity. In a preferred variation, the encoded polypeptide has FoxC2 transcription factor activity in mammalian cells, and highly preferably in human cells, such as human endothelial cells or endothelial precursor cells. In one variation, the encoded polypeptide comprises an amino acid sequence that is at least 90% identical to SEQ ID NO: 2 (human FoxC2) or to a fragment thereof, where the encoded polypeptide has FoxC2 transcription factor activity. Preferred embodiments are those which retain FoxC2 transcription factor activity in human endothelial cells that express the polypeptide. Encoded sequences that are at least 95%, 97%, 99% or 100% identical to SEQ ID NO: 2 are highly preferred. Vectors of the invention are useful for transforming or transfecting cells in vitro/ex vivo or in vivo as described herein. Vectors with suitable expression control sequences, such as promoters or enhancers, are specifically contemplated.

In preferred embodiments, polynucleotides of the invention further comprises additional sequences to facilitate the gene therapy. In one embodiment, a “naked” transgene encoding a polypeptide of the invention (i.e., a transgene without a viral, liposomal, or other vector to facilitate transfection) is employed for gene therapy. In this embodiment, the polynucleotide of the invention preferably comprises a suitable promoter and/or enhancer sequence (e.g., cytomegalovirus promoter/enhancer [Lehner et al., J. Clin. Microbiol., 29:2494 2502 (1991); Boshart et al., Cell, 41:521 530 (1985)]; Rous sarcoma virus promoter [Davis et al., Hum. Gene Ther., 4:151 (1993)]; Tie promoter [Korhonen et al., Blood, 86(5): 1828 1835 (1995)]; or simian virus 40 promoter) for expression in the target mammalian cells, the promoter being operatively linked upstream (i.e., 5′) of the polypeptide coding sequence. The polynucleotides of the invention also preferably further includes a suitable polyadenylation sequence (e.g., the SV40 or human growth hormone gene polyadenylation sequence) operably linked downstream (i.e., 3′) of the polypeptide coding sequence. The polynucleotides of the invention also preferably comprise a nucleotide sequence encoding a secretory signal peptide fused in frame with the polypeptide sequence. The secretory signal peptide directs secretion of the polypeptide of the invention by the cells that express the polynucleotide, and is cleaved by the cell from the secreted polypeptide. The signal peptide sequence can be that of another secreted protein, or can be a completely synthetic signal sequence effective to direct secretion in cells of the mammalian subject.

Such vectors are useful, e.g., for amplifying the polynucleotides in host cells to create useful quantities thereof. In preferred embodiments, the vector is an expression vector wherein the polynucleotide of the invention is operatively linked to a polynucleotide comprising an expression control sequence. Autonomously replicating recombinant expression constructs such as plasmid and viral DNA vectors incorporating polynucleotides of the invention are specifically contemplated. Expression control DNA sequences include promoters, enhancers, and operators, and are generally selected based on the expression systems in which the expression construct is to be utilized. Preferred promoter and enhancer sequences are generally selected for the ability to increase gene expression, while operator sequences are generally selected for the ability to regulate gene expression. Polyadenylation sequences also are contemplated. Expression vectors are useful for recombinant production of polypeptides of the invention. Expression constructs of the invention may also include sequences encoding one or more selectable markers that permit identification of host cells bearing the construct. Expression constructs may also include sequences that facilitate, and preferably promote, homologous recombination in a host cell. In some variations, vectors of the invention also include sequences necessary for replication in a host cell.

A preferred vector of the invention is a gene therapy vector that is useful for transforming or transfecting mammalian cells in vivo or ex vivo, to introduce the FoxC2 coding sequence in a manner that permits FoxC2 expression in the cells. Exemplary gene therapy vectors include replication deficient viral vectors described below in greater detail. Exemplary promoters for inclusion in FoxC2 gene therapy invention include constitutive promoters, such as CMV, or tissue-specific promoters, such as the Tie promoter. Preferred target cells are vessel cells, especially lymphatic endothelial and vascular endothelial cells.

Vectors also are useful for “gene therapy” treatment regimens, wherein a polynucleotide that encodes a polypeptide of the invention is introduced into a subject in need of treatment involving impaired lymphatic or arterial, or venous function, in a form that causes cells in the subject to express the polypeptide of the invention in vivo.

Another aspect of the invention is a host cell, including prokaryotic and eukaryotic cells, that is transformed or transfected (stably or transiently) with polynucleotides of the invention or vectors of the invention. Polynucleotides of the invention may be introduced into the host cell as part of a circular plasmid, or as linear DNA comprising an isolated protein coding region or a viral vector. Methods for introducing DNA into the host cell well known and routinely practiced in the art include transformation, transfection, electroporation, nuclear injection, or fusion with carriers such as liposomes, micelles, ghost cells, and protoplasts. As stated above, such host cells are useful for amplifying the polynucleotides and also for expressing the polypeptides of the invention encoded by the polynucleotide. Such host cells are useful in assays as described herein. For expression of polypeptides of the invention, any host cell is acceptable, including but not limited to bacterial, yeast, plant, invertebrate (e.g., insect), vertebrate, and mammalian host cells. For developing therapeutic preparations, expression in mammalian cell lines, especially human cell lines, is preferred. Use of mammalian host cells is expected to provide for such post-translational modifications (e.g., glycosylation, truncation, lipidation, and phosphorylation) as may be desirable to confer optimal biological activity on recombinant expression products of the invention. Glycosylated and non-glycosylated forms of polypeptides are embraced by the present invention. Similarly, the invention further embraces polypeptides described above that have been covalently modified to include one or more water soluble polymer attachments such as polyethylene glycol, polyoxyethylene glycol, or polypropylene glycol.

Another aspect of the invention is a mammalian (and more preferably a human) endothelial cell transformed or transfected with a FoxC2 expression construct. These cells are useful for ex vivo gene therapy. These cells are also useful for making synthetic valves of the invention.

In still another variation, endothelial cells or endothelial progenitor cells are transformed or transfected ex vivo with a transgene encoding a polypeptide with FoxC2 activity (e.g., transfected with a vector of the invention), and the transfected cells as administered to the mammalian subject. In some variations, the cells are administered directly, e.g., by intravenous or subcutaneous injection, preferably locally at a site in need of improved vessel function. In another variation, the cells are administered in conjunction with a medical device, such as an artificial valve, an endovascular stent, an extravascular collar, a vessel, or the like. Exemplary procedures for seeding a vascular graft with genetically modified endothelial cells are described in U.S. Pat. No. 5,785,965, incorporated herein by reference.

Aspects of the invention include materials and methods for transforming vascular or lymphatic cells (especially endothelial cells) with a FoxC2 and/or a Prox1 polynucleotide. The materials include vectors for transformation, and the transformed cells themselves. Cells may be transformed in vivo to improve in vivo valve formation or function. Cells also may be transformed ex vivo and (1) administered into a mammalian subject for the purpose of improving in vivo valve formation or function; (2) administered into a mammalian subject for the purpose of coating an implanted synthetic valve or other implantable device in the mammalian subject; or (3) used ex vivo to coat a synthetic valve or other implantable device prior to its implantation. Synthetic valves coated in this manner are themselves an aspect of the invention. In yet another variation, the synthetic valve is coated with the gene therapy vector to facilitate localized transfection of endothelial cells upon implantation. These aspects of the invention can be used to improve all synthetic valve technologies, including those in existence and later developed. A number of existing valve technologies that can be improved according to this invention are summarized below with reference to literature, all of which is incorporated by reference in its entirety.

Another aspect of the invention is a method of treating mammalian subjects with impaired lymphatic function comprising administering to a mammalian subject in need of treatment a composition comprising a gene therapy vector (such as a replication-deficient adenovirus) comprising a polynucleotide, wherein said composition is administered locally at the site in need of treatment to improve lymph flow, wherein said polynucleotide comprises a nucleotide sequence that encodes a polypeptide comprising an amino acid sequence at least 90% identical to a FoxC2 amino acid sequence, such as the human FoxC2 sequence of SEQ ID NO: 2, or fragment thereof sufficient to confer FoxC2 transcription factor activity, and wherein the coding sequence is operatively linked to a promoter to promote expression of the FoxC2 polypeptide in lymphatic vessel cells, wherein expression of said FoxC2 polypeptide in said lymphatic vessel cells improves the lymph flow of said lymphatic vessel.

Another aspect of the invention is an analogous gene therapy method for CVI, where expression of the transgene in venous endothelia improves venous function. Localized therapy is preferred.

Another aspect of the invention is a method of treating mammalian subjects with impaired valvular (lymphatic, arterial or venous) function comprising culturing the transfected FoxC2 endothelial cells with a synthetic valve for 1-3 days to ensure complete coverage of the synthetic valve with the transfected FoxC2 endothelial cells and implanting the synthetic valve into the vessel (lymphatic, venous, or arterial) with impaired valvular function.

In still another variation, the invention is a therapeutic or prophylactic method of improving lymphatic function comprising: isolating lymphatic endothelial cells or lymphatic endothelial progenitor cells from a subject selected from: subjects with impaired lymphatic function due to arterialization of lymphatic vessels, and subjects with a genetic risk for developing said impaired function; transforming or transfecting the cells ex vivo with a polynucleotide comprising a nucleic acid sequence that encodes a polypeptide comprising an amino acid sequence at least 90% identical to the Foxc2 amino acid sequence of SEQ ID NO: 2 or a fragment thereof, wherein the polypeptide is expressed in the cells and has Foxc2 transcription factor activity; and administering the transformed or transfected cells to the mammalian subject, in an amount effective to reduce or prevent lymphatic backflow in a lymphatic vessel, thereby improving lymphatic function. In preferred variations, the encoded polypeptide is more similar (e.g., 92, 94, 95, 96, 97, 98, 99, or 100% identical to the human FoxC2 sequence of SEQ ID NO: 2. In some variations, the method is practiced in animal models useful for studying human disease and animals of importance commercially or as pets (e.g., mice, rabbits, cats, dogs, pigs, primates, bovines). In other variations, the mammalian subject is human.

Similarly, another variation of the invention is a therapeutic or prophylactic method of improving venous function comprising: isolating vascular endothelial cells or vascular endothelial progenitor cells from a subject selected from: subjects with impaired vascular function, CVI, varicose veins, hemorrhoids, and subjects with a genetic risk for developing these problems; transforming or transfecting the cells ex vivo with a polynucleotide comprising a nucleic acid sequence that encodes a polypeptide comprising an amino acid sequence at least 90% identical to the Foxc2 amino acid sequence of SEQ ID NO: 2 or a fragment thereof, wherein the polypeptide is expressed in the cells and has Foxc2 transcription factor activity; and administering the transformed or transfected cells to the mammalian subject, in an amount effective to reduce or prevent venous backflow or improve venous flow, thereby improving venous function.

The cells may be administered by any route and device. For example, in some variations, a suspension of the cells is administered intravenously into the mammalian subject or injected subcutaneously, e.g., at or near a site of impaired lymphatic or venous function. In some variations, the cells are used to coat an implantable medical device. For example, the cells may be used to coat a synthetic valve, or an endovascular stent, and the administering step comprises implanting the synthetic valve or stent into a vessel in the mammalian subject. The cells may be used to coat a transplantable or synthetic section of vessel, where the administering comprises attaching the vessel (e.g., in a by-pass operation) in a subject.

In a related variation, the invention is a therapeutic or prophylactic method of improving lymphatic function comprising: administering a composition comprising a polynucleotide to a subject, wherein the subject is selected from: subjects with impaired lymphatic function due to arterialization of lymphatic vessels, and subjects with a genetic risk for developing said impaired function; and wherein the polynucleotide comprises a nucleic acid sequence that encodes a polypeptide comprising an amino acid sequence at least 90% identical to the Foxc2 amino acid sequence of SEQ ID NO: 2 or a fragment thereof, wherein the polypeptide has Foxc2 transcription factor activity. In some variations, the composition is administered locally at a site in need of treatment to improve lymph flow.

Similarly, the invention is a therapeutic or prophylactic method of improving venous function by administering such a composition to an individual in need thereof, such as an individual with CVI.

In some variations, the composition comprises an expression vector that comprises an expression control sequence operatively linked to the polynucleotide. By way of example, the expression vector comprises a member selected from the group consisting of: replication deficient adenoviral vectors, adeno-associated viral vectors, and lentivirus vectors. Preferably, the polynucleotide further comprises a promoter that promotes expression of the polynucleotide in a mammalian cell.

In still another aspect, the invention is an improvement to a synthetic valve for implantation in a lumen of a blood or lymphatic vessel, said improvement comprising coating a surface of the valve with endothelial cells, wherein the endothelial cells are transformed or transfected with a polynucleotide comprising a nucleotide sequence that encodes a polypeptide that comprises an amino acid sequence at least 90% identical to the Foxc2 amino acid sequence of SEQ ID NO: 2 or a fragment thereof, wherein the polypeptide is expressed in the cells and has Foxc2 transcription factor activity. The same improvement to other implantable synthetic, therapeutic, prosthetic, or other therapeutic devices for the vascular or lymphatic system is contemplated.

Another embodiment of the invention is an insertable or implantable medical device for the vascular system with is coated with, or contains, a gene therapy construct as described herein. For example, an extravascular collar or cuff, as described in International Patent Publication Nos. WO 98/20027 (PCT/GB97/03015) and WO 99/55315 (PCT/GB99/01310), is impregnated with, or containing a composition comprising the gene therapy construct of the invention is itself an aspect of the invention, as is a method of therapy involving implanting such a device around a vessel to improve lymphatic or venous function. These published patent applications are incorporated herein by reference in their entirety. In some embodiments, the device comprises a biodegradable matrix material, such as collagen.

In still another variation, the invention is an isolated endothelial cell or endothelial precursor cell transformed or transfected with a polynucleotide comprising a nucleotide sequence that encodes a polypeptide that comprises an amino acid sequence at least 90% identical to the Foxc2 amino acid sequence of SEQ ID NO: 2 or a fragment thereof, wherein the polypeptide is expressed in the cell and has Foxc2 transcription factor activity. Such cells can be administered for therapeutic purposes as described herein, either as a suspension or adhered to the surface of a medical implant. For example, the invention also is a medical device comprising: a synthetic valve or stent that is implantable in a mammalian vessel; and transformed or transfected endothelial cells on a surface of the synthetic valve or stent.

In still another variation, it is contemplated that the invention comprises the use of a gene therapy construct comprising other homeobox transcription factors, such as PROX1, in all materials and methods described herein. Thus variations of gene therapy methods described herein are contemplated, wherein the nucleotide sequence introduced encodes a polypeptides that is at least 90% identical to the PROX1 amino acid sequence of SEQ ID NO: 35 or a fragment thereof that retains PROX1 transcription factor activity.

Additional aspects of the invention are defined or summarized in the following numbered paragraphs:

1. A therapeutic or prophylactic method of improving lymphatic function comprising:
administering to a mammalian subject a composition comprising an inhibitor of arterialization of lymphatic vessels,
wherein the subject is selected from: subjects with impaired lymphatic function due to arterialization of lymphatic vessels, and subjects with a genetic risk for developing said impaired function.

2. The method of paragraph 1, wherein the subject is human.

3. The method of paragraph 1 or 2, wherein the subject has lymphedema from the impaired lymphatic function.

4. The method of paragraph 1 or 2, wherein the subject has lymphedema distichiasis.

5. The method of paragraph 1 or 2, wherein the impaired lymphatic function is diagnosed from abnormal lymphatic drainage, increased number of lymph nodes, lymph flowback, and a lymphatic capillary network that comprises smooth muscle cells associated with lymphatic cells.

6. The method of any one of paragraphs 1-5, wherein the subject has a Foxc2 mutation genotype.

7. The method of any one of paragraphs 1-6, wherein the subject has lymphatic vessels characterized by at least one arterialization indicator selected from the group consisting of:
(A) expression of a smooth muscle cell marker in lymphatic capillaries from the subject;
(B) tortuous and distended dermal lymphatic capillaries;
(C) smooth muscle cells associated with lymphatic vessels;
(D) pericytes associated with lymphatic vessels;
(E) lymphatic hyperplasia; and,
(F) expression in lymphatic vessels of a blood vessel basal lamina proteins.

8. The method of any one of paragraphs 1-7, wherein the subject has lymphatic vessels characterized by absent or dysfunctional lymphatic valves.

9. The method of any one of paragraphs 1-2 and 6-8, wherein the subject has a genetic risk for developing lymphedema, wherein said risk is diagnosed from determinations that the subject has one or more of the following: (A) at least one genetic relative with hereditary lymphedema; (B) a Foxc2 mutation genotype; (C) a Foxc1 mutation genotype; (D) distichiasis; (E) smooth muscle cells covering lymphatic capillaries; (F) hyperplastic cutaneous lymphatic vessel density; (G) PDGFR-beta-expressing cells associated with lymphatic vessels; (H) PDGF-B overexpression in lymphatic vessels (I) smooth muscle cell association with lymphatic capillaries; (J) pericyte association with lymphatic capillaries; and (K) expression of blood vessel basal lamina components in lymphatic vessels.

10. The method of paragraph 9, wherein said risk is further diagnosed from an absence of or dysfunctional lymphatic valves in lymphatic vessels of the subject.

11. The method of paragraph 9, wherein the subject has at least one genetic relative with hereditary lymphedema and has a Foxc2 mutation genotype.

12. The therapeutic method according to any one of paragraphs 1-11, wherein the inhibitor of arterialization is administered in an amount effective to reduce edema, reduce pain from edema, increase limb function, increase FOXC2 expression in lymphatic endothelia, reduce PDGF-B expression in lymphatic endothelia, or reduce SMC association with lymphatic capillaries, or combinations thereof.

13. The therapeutic method according to any one of paragraphs 1-11, wherein the inhibitor of arterialization is administered in an amount effective to reduce lymphatic backflow as judged by lymphoscintigraphy.

14. The prophylactic method according to any one of paragraphs 1-2 and 6-11, wherein the inhibitor of arterialization is administered in an amount effective to inhibit smooth muscle cell or pericyte association with lymphatic capillaries in the subject.

15. Use of an inhibitor of arterialization to treat or prevent impaired lymphatic function.

16. Use of an inhibitor of arterialization in the manufacture of a medicament to treat or prevent impaired lymphatic function.

17. The method or use of any one of paragraphs 1-16, wherein the inhibitor of arterialization is an inhibitor of PDGFR-beta activity.

18. The method or use of paragraph 17, wherein the inhibitor of PDGFR-beta activity is selected from the group consisting of:
(A) inhibitors of PDGFR-beta expression;
(B) inhibitors of PDGF-B stimulation of PDGFR-beta;
(C) inhibitors of PDGF-B expression;
(D) inhibitors of PDGFR-beta signaling; and
(E) combinations thereof.

19. The method or use of paragraph 18, wherein the inhibitor of PDGFR-beta activity is an inhibitor of PDGFR-beta expression selected from the group consisting of:
(A) an antisense molecule directed to PDGFR-beta;
(B) an interfering RNA (RNAi) directed to PDGFR-beta;
(C) an aptamer that binds PDGFR-beta RNA;
(D) a ribozyme directed to PDGFR-beta; and
(E) combinations thereof.

20. The method or use of paragraph 18, wherein the inhibitor of PDGFR-beta activity is an inhibitor of PDGF-B stimulation of PDGFR-beta selected from the group consisting of:
(A) an antibody substance that binds to the extracellular domain of PDGFR-beta and inhibits PDGF binding;
(B) an antibody substance that binds to PDGF-B and inhibits the PDGF-B from binding or activating PDGFR-beta;
(C) a polypeptide comprising a soluble fragment of PDGFR-beta, wherein the polypeptide and fragment bind PDGF-B;
(D) a fragment of PDGF-B that binds and fails to stimulate PDGFR-beta;
(E) a polypeptide comprising a soluble fragment of PDGFR-alpha, wherein the polypeptide and fragment bind PDGF-B; and
(F) combinations thereof.

21. The method or use of paragraph 18, wherein the inhibitor of PDGFR-beta activity is an inhibitor of PDGF-B expression selected from the group consisting of:
(A) an antisense molecule directed to PDGF-B;
(B) interfering RNA (RNAi) directed to PDGF-B;
(C) an aptamer that binds PDGF-B RNA;
(D) a ribozyme directed to PDGF-B; and
(E) combinations thereof.

22. The method or use of paragraph 18, wherein the inhibitor of PDGFR-beta activity is an inhibitor of PDGFR-beta signaling.

23. The method or use of paragraph 22, wherein the inhibitor of PDGFR-beta activity is a tyrosine kinase inhibitor.

24. The method or use of paragraph 23, wherein the tyrosine kinase inhibitor is imatinib mesylate.

25. The method or use of any one of paragraphs 1-24, wherein the inhibitor of arterialization is administered subcutaneously at a site of edema.

26. A method or use according to any one of paragraphs 1-25, further comprising administering to the subject a growth factor product selected from the group consisting of vascular endothelial growth factor C (VEGF-C) protein products, vascular endothelial growth factor D (VEGF-D) protein products, VEGF-C gene therapy products, and VEGF-D gene therapy protein products.

27. A method or use according to any one of paragraphs 1-26, further comprising administering to the subject a smooth muscle cell relaxant.

28. A therapeutic or prophylactic method of treating arterialization of lymphatic vessels in a mammalian subject, comprising:
providing isolated lymphatic endothelial cells or lymphatic endothelial progenitor cells;
transforming or transfecting the cells ex vivo with a polynucleotide comprising a nucleotide sequence that encodes an inhibitor of PDGF expression; and
administering the transformed or transfected cells to the mammalian subject.

29. A method according to paragraph 28, wherein the transformed or transfected cells are administered locally at a site of edema in the subject.

30. A therapeutic or prophylactic method of improving lymphatic function comprising:
administering to a mammalian subject a composition comprising a smooth muscle relaxant;
wherein the subject is identified as having arterialization of lymphatic vessels; and
wherein the smooth muscle relaxant is administered in an amount effective to improve lymphatic function in the subject.

31. The method of paragraph 30, wherein the composition is administered locally at a site of edema in the subject.

32. A method of screening for an agent to improve lymphatic function in mammalian subjects having arterialization of lymphatic vessels, comprising steps of:
contacting arterialized lymphatic tissue with a test agent, wherein the arterialized lymphatic tissue comprises lymphatic endothelial cells associated with pericytes or smooth muscle cells;
determining if the test agent causes dissociation of lymphatic endothelial cells from pericytes or smooth muscle cells, wherein a test agent that causes the dissociation is selected as an agent to improve lymphatic function.

33. The method of paragraph 32, wherein the lymphatic tissue is obtained from an organism with a Foxc2 mutation.

34. The method of paragraph 32, further comprising a step of contacting blood vessels with the test agent, and selecting a test agent that preferentially causes smooth muscle dissociation form lymphatic vessels compared to dissociation of smooth muscle cells from blood vessels.

35. A therapeutic or prophylactic method of improving lymphatic function comprising:
isolating lymphatic endothelial cells or lymphatic endothelial progenitor cells from a subject selected from: subjects with impaired lymphatic function due to arterialization of lymphatic vessels, and subjects with a genetic risk for developing said impaired function;
transforming or transfecting the cells ex vivo with a polynucleotide comprising a nucleic acid sequence that encodes a polypeptide comprising an amino acid sequence at least 90% identical to the Foxc2 amino acid sequence of SEQ ID NO: 2 or a fragment thereof, wherein the polypeptide is expressed in the cells and has Foxc2 transcription factor activity; and
administering the transformed or transfected cells to the mammalian subject, in an amount effective to reduce or prevent lymphatic backflow in a lymphatic vessel, thereby improving lymphatic function.

36. A method according to paragraph 35, wherein the encoded polypeptide comprises the amino acid sequence of SEQ ID NO: 2.

37. A method according to paragraph 35, wherein a suspension of the cells is administered intravenously into the mammalian subject.

38. A method according to paragraph 35, further comprising a step of coating a synthetic valve with the transformed or transfected cells, wherein the administering comprises implanting the synthetic valve into a vessel in the mammalian subject.

39. A therapeutic or prophylactic method of improving lymphatic function comprising:
administering a composition comprising a polynucleotide to a subject, wherein the subject is selected from: subjects with impaired lymphatic function due to arterialization of lymphatic vessels, and subjects with a genetic risk for developing said impaired function; and wherein the polynucleotide comprises a nucleic acid sequence that encodes a polypeptide comprising an amino acid sequence at least 90% identical to the Foxc2 amino acid sequence of SEQ ID NO: 2 or a fragment thereof, wherein the polypeptide has Foxc2 transcription factor activity.

40. The method of paragraph 39, wherein said composition is administered locally at a site in need of treatment to improve lymph flow.

41. The method of paragraph 39, wherein the composition comprises an expression vector that comprises an expression control sequence operatively linked to the polynucleotide.

42. The method of paragraph 41, wherein the expression vector comprises a member selected from the group consisting of: replication deficient adenoviral vectors, adeno-associated viral vectors, and lentivirus vectors.

43. The method of paragraph 39, wherein the polynucleotide further comprises a promoter that promotes expression of the polynucleotide in a mammalian cell.

44. An improvement to a synthetic valve for implantation in a lumen of a blood or lymphatic vessel; said improvement comprising coating a surface of the valve with endothelial cells, wherein the endothelial cells are transformed or transfected with a polynucleotide comprising a nucleotide sequence that encodes a polypeptide that comprises an amino acid sequence at least 90% identical to the Foxc2 amino acid sequence of SEQ ID NO: 2 or a fragment thereof, wherein the polypeptide is expressed in the cells and has Foxc2 transcription factor activity.

45. An isolated endothelial cell or endothelial precursor cell transformed or transfected with a polynucleotide comprising a nucleotide sequence that encodes a polypeptide that comprises an amino acid sequence at least 90% identical to the Foxc2 amino acid sequence of SEQ ID NO: 2 or a fragment thereof, wherein the polypeptide is expressed in the cell and has Foxc2 transcription factor activity.

46. A medical device comprising: a synthetic valve that is implantable in a mammalian vessel; and endothelial cells according to paragraph 45 on a surface of the synthetic valve.

47. A medical device comprising an endovascular stent that is implantable in a mammalian vessel, and endothelial cells according to paragraph 43 on a surface of the stent.

48. A therapeutic or prophylactic method of improving venous flow comprising:
isolating venous endothelial cells or venous endothelial progenitor cells from a mammalian subject selected from: subjects with impaired venous flow due to absent or dysfunctional venous valves, and subjects with a genetic risk for developing said impaired flow;
transforming or transfecting the cells ex vivo with a polynucleotide comprising a nucleic acid sequence that encodes a polypeptide comprising an amino acid sequence at least 90% identical to the FOXC2 amino acid sequence of SEQ ID NO: 2 or a fragment thereof, wherein the polypeptide is expressed in the cells and has FOXC2 transcription factor activity; and
administering the transformed or transfected cells to the mammalian subject, in an amount effective to reduce or prevent venous backflow in a blood vessel, thereby improving venous function.

49. A method according to paragraph 48, wherein the mammalian subject is human.

50. A method according to paragraph 48 or 49 wherein the subject has chronic venous insufficiency.

51. A method according to paragraph 48 or 49, wherein the impaired venous function is diagnosed from the existence of varicose veins and/or hemorrhoids.

52. A method according to any one of paragraphs 48-51, wherein the subject has a FoxC2 mutation genotype.

53. A method according to paragraph 48, wherein the encoded polypeptide comprises the amino acid sequence of SEQ ID NO: 2.

54. A method according to paragraph 48, wherein a suspension of the cells is administered intravenously into the mammalian subject.

55. A method according to paragraph 48, further comprising a step of coating a synthetic valve with the transformed or transfected cells, wherein the administering comprises implanting the synthetic valve into a vein in the mammalian subject.

56. A therapeutic or prophylactic method of improving venous flow comprising:
administering a composition comprising a polynucleotide to a subject, wherein the subject is selected from: subjects with impaired venous flow due to the absence of or dysfunctional venous valves, and subjects with a genetic risk for developing said impaired function; and wherein the polynucleotide comprises a nucleic acid sequence that encodes a polypeptide comprising an amino acid sequence at least 90% identical to the FOXC2 amino acid sequence of SEQ ID NO: 2 or a fragment thereof, wherein the polypeptide has Foxc2 transcription factor activity.

57. The method of paragraph 56, wherein said composition is administered locally at a site in need of treatment to improve venous flow.

58. The method of paragraph 56, wherein the composition comprises an expression vector that comprises an expression control sequence operatively linked to the polynucleotide.

59. The method of paragraph 58, wherein the expression vector comprises a member selected from the group consisting of: replication deficient adenoviral vectors, adeno-associated viral vectors, and lentivirus vectors.

60. The method of paragraph 56, wherein the polynucleotide further comprises a promoter that promotes expression of the polynucleotide in a mammalian cell. Additional features and variations of the invention will be apparent to those skilled in the art from the entirety of this application, including the detailed description, and all such features are intended as aspects of the invention. It should be understood, however, that the detailed description and the specific examples, while indicating preferred embodiments of the invention, are given by way of illustration only, because various changes and modifications within the spirit and scope of the invention will become apparent to those skilled in the art from this detailed description.

Moreover, features of the invention described herein can be recombined into additional embodiments that also are intended as aspects of the invention, irrespective of whether the combination of features is specifically mentioned above as an aspect or embodiment of the invention. Also, only those limitations that are described herein as critical to the invention should be viewed as such; variations of the invention lacking features that have not been described herein as critical are intended as aspects of the invention.

With respect to aspects of the invention that have been described as a set or genus, every individual member of the set or genus is intended, individually, as an aspect of the invention, even if, for brevity, every individual member has not been specifically mentioned herein. When aspects of the invention that are described herein as being selected from a genus, it should be understood that the selection can include mixtures of two or more members of the genus.

In addition to the foregoing, the invention includes, as an additional aspect, all embodiments of the invention narrower in scope in any way than the variations specifically described herein. Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE DRAWING

FIG. 1 is a graph depicting data indicating that the proportion of smooth muscle actin positive (SMA+) lymphatic vessels is significantly higher in the foot skin of in LD patients compared to individuals without FOXC2 mutations. A, affected, N, unaffected.

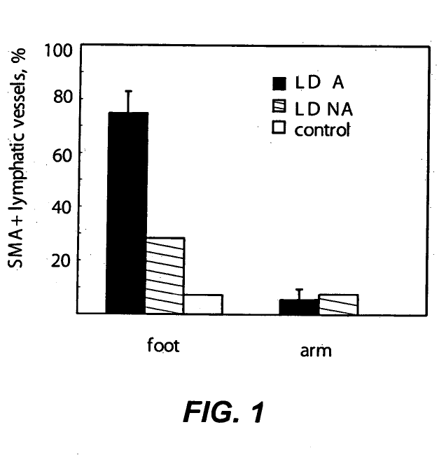

### DETAILED DESCRIPTION OF THE INVENTION

The present invention addresses heretofore unmet needs by providing materials and methods for diagnosis of, and prophylactic and therapeutic treatments for, certain heritable lymphedemas or acquired lymphedemas with similar characteristics. The present invention is based, in part, on the finding that FOXC2 is essential for establishing a smooth muscle cell-free lymphatic capillary network and that FOXC2 cooperates with Vegfr-3 in establishing a smooth muscle cell free lymphatic network. The present invention also is based, in part, on the discovery that Foxc2 is essential for the development of lymphatic valves and that persistent expression of Foxc2 suggests a role in the maintenance of their function. The present invention also is based, in part, on the discovery of biological pathway targets for intervention to alleviate this disorder.

More particularly, the data described herein show that FOXC2 is expressed in the lymphatic endothelium, and Foxc2^{-/-} mice and mice heterozygous for Foxc2 and the lymphatic endothelial receptor Vegfr-3 display abnormal lymphatic vascular patterning and increased pericyte/smooth muscle cell investment of lymphatic capillaries. Moreover, an abnormally high proportion of skin lymphatic vessels also are covered with smooth muscle cells in human subjects with lymphedema-distichiasis. It is also demonstrated herein that in the absence of Foxc2, expression of the pericyte/smooth muscle cell chemoattractant known as platelet-derived growth factor-B (PDGF-B) is induced in the lymphatic endothelium. These data indicate that an abnormal interaction between the lymphatic endothelial cells and pericytes/smooth muscle cells underlies the pathogenesis of lymphedema-distichiasis. Methods and compositions for exploiting these findings in the treatment of lymphedema in general and lymphedema-distichiasis in particular, are described here.

In contrast to blood vessels, healthy normal lymphatic vessels, e.g., skin lymphatic vessels, do not produce PDGF-B, a growth factor essential for the recruitment of PCs to blood vasculature. However, lymphatic production of PDGF-B was observed in mice where Foxc2 is inactivated. These results suggest that FOXC2 is required for establishing a SMC-free lymphatic capillary network through the suppression of PDGF-B expression in the lymphatics. The observations from studies performed in Foxc2 knockout mouse models have been supported by the similar findings in LD patients harboring Foxc2 mutations. Thus, the present invention describes both the discovery of an abnormal association between the lymphatic vessels and PC/SMCs, and a potential molecular mechanism underlying this association. The smooth muscle cells provide an explanation for abnormal lymphatic patterning and function that result in the clinical manifestations of this disease. These findings are useful in designing methods of treatment of not only LD, but other types of lymphedema or lymphatic arterialization, as well as for designing methods of identifying agents that will augment, enhance, stimulate, increase, or otherwise upregulate the expression or activity of FOXC2 and/or decrease, down-regulates or otherwise inhibit the activity or expression of PDGF-B or its interaction with its receptor PDGFR-B on PC/SMC. Such an increase in FOXC2 expression and/or activity or decrease in PDGF-B expression/activity has an indication in ameliorating lymphedemas that are caused by mutations in Foxc2 as well as more broadly, lymphedemas that have a similar phenotype characterized by abnormal PC/SMC investment of lymphatic capillaries.

I. Identification of Some Genes and Protein Sequences of Interest to the Invention

A number of genes and proteins are of particular relevance to the invention, including FOXC2, VEGF-C, VEGF-D, VEGFR-3, the PDGF growth factors (PDGF-A, -B, -C, and -D), and PDGF receptors (PDGFR-alpha and PDGFR-beta). For each of these, wildtype (normal) human sequences are known, as are sequences from other species. For convenience, a number of sequences are appended to the sequence listing of the application, including human FOXC2 cDNA and protein (SEQ ID NOs: 1-2); murine FOXC2 cDNA and protein (SEQ ID NOs: 3-4); human VEGF-C cDNA and protein (SEQ ID NOs: 5-6); human VEGF-D cDNA and protein (SEQ ID NOs: 7-8); human VEGFR-3 cDNA and protein, long form (SEQ ID NOs: 9-10) and short form (SEQ ID NOs: 11-12); human PDGFR-alpha (SEQ ID NOs:13-14); human PDGFR-beta (SEQ ID NOs: 15-16); human PDGF-A (SEQ ID NOs: 17-18); human PDGF-B (SEQ ID NOs: 19-20); human PDGF-C (SEQ ID NOs: 21-22); human PDGF-D (SEQ ID NOs: 23-24) and human PROX1 (SEQ ID NOs: 34-35). For at least some of these genes, including Foxc2 and VEGFR-3, mutant alleles associated with lymphedema have been reported. Exemplary sequences also are reported in sequence databases, including Genbank.

| | SEQ ID | |
|---|---|---|
| Protein/Gene | NOS: | Related Database Sequences |
| human FOXC2 | 1 and 2 | GenBank Acc. No. NM_005251 |
| murine FOXC2 | 3 and 4 | |
| human VEGF-C | 5 and 6 | GenBank Acc. Nos. AF466147; |
| | | BC063685; NM_174488; |
| | | NM_053653; NM_005429; |
| | | NM_009506; NT_039460; NW_047473; |
| | | BC035212; AF432867; AF432866; |
| | | AF432865; and AH011187. |
| human VEGF-D | 7 and 8 | |
| human VEGFR-3 | 9 and 10 | GenBank Acc. Nos. NM_182925; |
| (long form) | | NM_002020; NM_053652; |
| human VEGFR-3 | 11 and 12 | NM_008029; AY233383; |
| (short form) | | AY233382; AF402786; |
| | | AF402785; AF041795; and AF030379 |
| human PDGFR-α | 13 and 14 | |
| human PDGFR-β | 15 and 16 | GenBank Acc. No. NM_008809 |
| human PDGF-A | 17 and 18 | |
| human PDGF-B | 19 and 20 | GenBank Acc. Nos. NM_033016; |
| | | NM_002608; NM_011057; M12783, |
| | | NM_008809; and BC023427 |
| human PDGF-C | 21 and 22 | |
| human PDGF-D | 23 and 24 | |
| human PROX-1 | 34 and 35 | Genbank Acc. No. NM_002763 |

The platelet-derived growth factor (PDGF) subfamily comprises thus far four family members: PDGF-A, PDGF-B, PDGF-C, and PDGF-D. These ligands bind and activate, with distinct selectivity, dimeric complexes of the receptor tyrosine kinases PDGFR-α and PDGFR-β. [Heldin, C. H. & Westermark, B. Physiol Rev 79, 1283-1316 (1999).] PDGF-A and PDGF-B can homodimerize or heterodimerize to produce three different isoforms: PDGF-AA, PDGF-AB, or PDGF-BB. PDGF-A is only able to bind the PDGF α-receptor (PDGFR-α including PDGR-α/α homodimers). PDGF-B can bind both the PDGFR-α and a second PDGF receptor (PDGFR-β). More specifically, PDGF-B can bind to PDGFR-α/α and PDGFR-β/β homodimers, as well as PDGFR-α/β heterodimers. PDGF-C and PDGF-D exist as homodimers. PDGF-C binds PDGR-α/α homodimers and PDGF-D binds PDGFR-β/β homodimers, and both have been reported to bind PDGFR-α/β heterodimers.

The PDGFs regulate cell proliferation, cell survival and chemotaxis of many cell types in vitro (reviewed in [Heldin et al., Biochimica et Biophysica Acta 1378:F79-113 (1998); Carmeliet P et al. Nature 380, 435-9 (1996); Hellström, M. et al. J Cell Biol 153, 543-53. (2001).]. In vivo, the PDGF proteins exert their effects in a paracrine manner since they often are expressed in epithelial (PDGF-A) or endothelial (PDGF-B) cells in close apposition to the PDGF receptor-expressing mesenchyme [reviewed in Alitalo et al., Int Rev Cytology 172:95-127 (1997)]. Overexpression of the PDGFs has been observed in several pathological conditions, including malignancies, atherosclerosis, and fibroproliferative diseases. In tumor cells and cell lines grown in vitro, co-expression of the PDGFs and PDGF receptors generates autocrine loops, which are important for cellular transformation [Betsholtz et al., Cell 39:447-57 (1984); Keating et al., Science 239:914-6 (1988)].

The importance of the PDGFs as regulators of cell proliferation and cell survival is well illustrated by recent gene targeting studies in mice. Homozygous null mutations for either PDGF-A or PDGF-B are lethal in mice. Approximately 50% of the homozygous PDGF-A deficient mice have an early lethal phenotype, while the surviving animals have a complex postnatal phenotype with lung emphysema due to improper alveolar septum formation, and a dermal phenotype characterized by thin dermis, misshapen hair follicles, and thin hair. PDGF-A is also required for normal development of oligodendrocytes and subsequent myelination of the central nervous system. The PDGF-B deficient mice develop renal, hematological and cardiovascular abnormalities; where the renal and cardiovascular defects, at least in part, are due to the lack of proper recruitment of mural cells (vascular smooth muscle cells, pericytes or mesangial cells) to blood vessels.

PDGF-AA and -BB are the major mitogens and chemoattractants for cells of mesenchymal origin, but have no, or little effect on cells of endothelial lineage, although both PDGFR-α and -β are expressed on endothelial cells (EC). PDGF-BB and PDGF-AB have been shown to be involved in the stabilization/maturation of newly formed vessels [Isner, J. M. Nature 415, 234-9. (2002); Vale, P. R., Isner, J. M. & Rosenfield, K. J Interv Cardiol 14, 511-28 (2001); Heldin, C. H. & Westermark, B. Physiol Rev 79, 1283-1316 (1999); Betsholtz, C., Karlsson, L. & Lindahl, P. Bioessays 23, 494-507. (2001)]. Other data however, showed that PDGF-BB and PDGF-AA inhibited bFGF-induced angiogenesis in vivo via PDGFR-α signaling. PDGF-AA is among the most potent stimuli of mesenchymal cell migration, but it either does not stimulate or it minimally stimulates EC migration. In certain conditions, PDGF-AA even inhibits EC migration [Thommen, J Cell Biochem. 1997 Mar. 1; 64(3):403-13; De Marchis, F., et al., Blood 99:2045-53 (2002); Cao, R., et al., FASEB. J. 16:1575-83 (2002).] Moreover, PDGFR-α has been shown to antagonize the PDGFR-β-induced SMC migration Yu, J., et al., Biochem. Biophys. Res. Commun. 282:697-700 (2001) and neutralizing antibodies against PDGF-AA enhance smooth muscle cell (SMC) migration (Palumbo, R., et al., Arterioscler. Thromb. Vasc. Biol. 22:405-11 (2002). Thus, the angiogenic/arteriogenic activity of the PDGFs, especially when signaling through PDGFR-α, has been controversial and enigmatic.

Exemplary PDGF-A and -B polypeptides for use in the invention have been described in U.S. Pat. No. 5,605,816 (PDGF-A and A/B heterodimers); U.S. Pat. No. 4,889,919 (PDGF-A homodimers); U.S. Pat. No. 5,759,815 (recombinant production of PDGF-A or -B in prokaryotes and formation of various dimers); U.S. Pat. No. 5,889,149 (PDGF-AB isoforms); U.S. Pat. Nos. 4,845,075 and 5,428,010 and 5,516,896 (PDGF-BB homodimers); U.S. Pat. Nos. 5,272,064 and 5,512,545 (PDGF-B analogues); U.S. Pat. No. 5,905,142 (protease-resistant PDGF-B analogues); and U.S. Pat. Nos. 5,128,321 and 5,498,600 and 5,474,982 (PDGF-A/B mosaics).

PDGF-C polypeptides and polynucleotides were characterized by Eriksson et al. in International Patent Publication No. WO 00/18212, U.S. Patent Application Publication No. 2002/0164687 A1, and U.S. patent application Ser. No. 10/303,997 [published as U.S. Pat. Publ. No. 2003/0211994]. PDGF-D polynucleotides and polypeptides were characterized by Eriksson, et al. in International Patent Publication No. WO 00/27879 and U.S. Patent Application Publication No. 2002/0164710 A1. A distinction between these polypeptides and PDGF-A and -B is that PDGF-C and -D each possess an amino-terminal CUB domain that can be proteolytically cleaved to yield a biologically active (receptor binding) carboxy-terminal domain with sequence homology to other PDGF family members.

PDGF-CC is secreted as an inactive homodimer of approximately 95 kD. Upon proteolytic removal of the CUB domain, PDGF-CC is capable of binding and activating its receptor, PDGFR-α [Li, X. & Eriksson, U., Cytokine & Growth Factor Reviews 244:1-8 (2003)]. In cells co-expressing both PDGFR-α and -β, PDGF-CC may also activate the PDGFR-α/β heterodimer, but not the PDGFR-β/β homodimer [Cao, R., et al., FASEB. J 16:1575-83. (2002); Gilbertson, D. G., et al., J. Biol. Chem. 10:10 (2001)].

Active PDGF-CC is a potent mitogen for fibroblast and vascular smooth muscle cells [Li, et al., Nat. Cell. Biol. 2:302-09 (2000); Cao, et al., FASEB. J 16:1575-83 (2002); Uutela, et al., Circulation 103:2242-7 (2001)]. Both PDGF-AA and PDGF-CC bind PDGFR-α, but only PDGF-CC potently stimulates angiogenesis in mouse cornea pocket and chick chorioallanoic membrane (CAM) assays [Cao, et al., FASEB. J 16:1575-83 (2002)]. PDGF-CC also promotes wound healing by stimulating tissue vascularization [Gilbertson, et al., J. Biol. Chem. 10:10 (2001)].

VEGF family members include VEGF-A, VEGF-B, VEGF-C, VEGF-D and P1GF [Li, X. and U. Eriksson, “Novel VEGF Family Members: VEGF-B, VEGF-C and VEGF-D,” Int. J. Biochem. Cell. Biol., 33(4):421-6 (2001)).]

VEGF-C is originally expressed as a larger precursor protein, prepro-VEGF-C, having extensive amino- and carboxy-terminal peptide sequences flanking a VEGF Homology Domain (VHD), with the C-terminal peptide containing tandemly repeated cysteine residues in a motif typical of Balbiani ring 3 protein. Prepro-VEGF-C undergoes extensive proteolytic maturation involving the successive cleavage of a signal peptide, the C-terminal pro-peptide, and the N-terminal pro-peptide. Secreted VEGF-C protein consists of a non-covalently-linked homodimer, in which each monomer contains the VHD. The intermediate forms of VEGF-C produced by partial proteolytic processing show increasing affinity for the VEGFR-3 receptor, and the mature protein is also able to bind to the VEGFR-2 receptor. [Joukov, et al., EMBO J, 16(13):3898-3911 (1997).] It has also been demonstrated that a mutant VEGF-C, in which a single cysteine at position 156 is either substituted by another amino acid or deleted, loses the ability to bind VEGFR-2 but remains capable of binding and activating VEGFR-3 [International Patent Publication No. WO 98/33917]. In mouse embryos, VEGF-C mRNA is expressed primarily in the allantois, jugular area, and the metanephros. [Joukov, et al., J. Cell. Physiol. 173:211-15 (1997)].

VEGF-C is involved in the regulation of lymphatic angiogenesis: when VEGF-C was overexpressed in the skin of transgenic mice, a hyperplastic lymphatic vessel network was observed, suggesting that VEGF-C induces lymphatic growth [Jeltsch et al., Science, 276:1423-1425 (1997)]. Continued expression of VEGF-C in the adult also indicates a role in maintenance of differentiated lymphatic endothelium [Ferrara, J. Mol. Med. 77:527-543 (1999)]. In addition, VEGF-C shows angiogenic properties: it can stimulate migration of bovine capillary endothelial (BCE) cells in collagen and promote growth of human endothelial cells. [See, e.g., International Patent Publication No. WO 98/33917, incorporated herein by reference.]

U.S. Pat. Nos. 6,645,933, 6,403,088, 6,361,946, 6,245,530, 6,221,839 and U.S. Pat. No. 6,130,071, each incorporated herein by reference in its entirety, provide teachings of VEGF-C protein and gene sequences, mutants thereof, and methods of making and using the same. The VEGF-C Cys156 deletion/replacement mutant described herein and in these documents has particular relevance to lymphedema treatment due to its specificity for VEGFR-3. U.S. Pat. Nos. 5,776,755 and 6,107,046 incorporated herein by reference, providing teachings of VEGFR-3 (also known as FLT4) protein and gene sequences. VEGFR-3, and VEGFR-2, are able to bind both VEGF-C and VEGF-D.

VEGF-D is structurally and functionally most closely related to VEGF-C. [See International Patent Publ. No. WO 98/07832, incorporated herein by reference]. Like VEGF-C, VEGF-D is initially expressed as a prepro-peptide that undergoes N-terminal and C-terminal proteolytic processing, and forms non-covalently linked dimers. VEGF-D stimulates mitogenic responses in endothelial cells in vitro. During embryogenesis, VEGF-D is expressed in a complex temporal and spatial pattern, and its expression persists in the heart, lung, and skeletal muscles in adults. Isolation of a biologically active fragment of VEGF-D designated VEGF-DΔNΔC, is described in International Patent Publication No. WO 98/07832, incorporated herein by reference. VEGF-DANAC consists of amino acid residues 93 to 201 of VEGF-D linked to the affinity tag peptide FLAG®. VEGF-D protein and gene and uses thereof are taught in U.S. Pat. Nos. 6,235,713; 6,383,484, and 6,689,580, incorporated herein by reference.

II. Diagnosing Lymphedema or Chronic Venous Insufficiency

The present application is directed in certain aspects to methods of therapeutically or prophylacticly treating lymphedema, and in particular methods of treating a subject having LD, or genetically predisposed to develop LD. This section describes materials and methods for identifying subjects for such treatment by diagnosing LD or genetic predisposition for LD, which is especially valuable for genetic counseling and prophylactic treatment. Such diagnoses may be genetic diagnoses from the subject and family members and/or may involve physical examination of the subject and consideration of family history. Lymphedema with lymphatic vessel arterialization, characterized, e.g., by the SMC/PC and/or PDGF-B/PDGFR-B pathology described herein, may be amendable to treatment as described herein even if no Foxc2 mutation is present. Based on preliminary studies in mice, similar lymphedema pathology may be attributable to Foxc1 mutations as well. Other hereditary lymphedemas are caused by a mutation in VEGFR-3.

A. Genetic Diagnosis

Numerous mutations in Foxc2 that cause lymphedema have been described in the literature. See, e.g., Fang et al., Am. J. Hum. Gen. 67, 1382-1388, 2000; Finegold et al., Hum. Mol. Gen. 10, 1185-1189, 2001; Erickson et al., J. Med. Genet., 38, 761-766, 2001; and Bell et al., Hum. Gen. 108, 546-551, 2001, all incorporated herein by reference for those teachings. These mutations include, e.g., a mutation at nucleotide location (according to the sequence of NM_{—}005251) 232 which includes a 14 base-pair duplication insertion that terminates the encoded protein at residue 100; a C→T mutation at nucleotide 252 that produces a Q→STOP codon at amino acid position 84; a deletion of A at nucleotide 505 that terminates the protein at amino acid 202; an insertion of C at nucleotide 589 that terminates the protein at amino acid 463; and insertion of C at nucleotide 609 that terminates the protein at amino acid 463; a 19 base pair deletion beginning at nucleotide 902 that terminates the protein at amino acid 330; a deletion of T at nucleotide 902 that terminates the protein at amino acid 336; a 7 base pair duplication-insertion at nucleotide 937 that terminates the protein at amino acid 463; an insertion of T at nucleotide 983 that terminates the protein at amino acid 463 and a 16 base-pair deletion beginning at nucleotide 1238 that terminates the protein at amino acid 427. In addition, LD also has been shown to be associated with a number of polymorphisms. For example, a C→A change in the 5′ proximal promoter region of FOXC2 (position −966), a C→T polymorphism at nucleotide 108 and a C→T polymorphism at nucleotide 1395 are all associated with LD.

Any Foxc2 mutation that causes deletion or premature termination or alternative splicing to cause deletion of the FOXC2 DNA binding domain (SEQ ID NO: 2, amino acids 68-178) or C-terminal domain (amino acids 178-500) is expected to result in an LD phenotype. Individuals to be tested for mutations in Foxc2 include patients that have symptoms of lymphedema, and unaffected relatives, especially children, grandchildren, or great-grandchildren of individuals diagnosed with lymphedema. The presence of a Foxc2 mutation will identify those individuals that have LD and are amenable to treatment methods of the present invention. Subjects that do not manifest lymphedema may be screened to ensure that the subject is not at risk for developing LD, particularly where a family history of LD exists. Symptoms of LD are not usually observed until the age of puberty. However, a causative mutation in Foxc2 may be identified any time after conception or birth. A genetic screen for such mutations will reveal whether such individuals may benefit from the methods of the present invention, including prophylactic therapy to inhibit or prevent LD manifestations.

Methods of diagnosing a condition or disease associated with an aberration in a known polynucleotide sequence, by screening the alleles of a subject for such a mutation, are well known to those of skill in the art. In the context of the present application, the polynucleotide sequence encoding FOXC2 may be used for the diagnosis of LD where a mutation in the FOXC2 sequence (relative to normal Foxc2 sequences from the same species) is indicative of LD. A differential analysis also may be performed where the subject being diagnosed initially is referred for diagnosis because the subject manifests the symptoms of a lymphedema. As lymphedema also results from mutations in the sequence of VEGFR-3, determining the presence or absence of mutation in VEGFR-3 should reveal whether the lymphedema is caused by a mutation in VEGFR-3 or a mutation in FOXC2 or other causes. (International Patent Publication No. WO 00/58511, “SCREENING AND THERAPY FOR LYMPHATIC DISORDERS INVOLVING THE FLT4 RECEPTOR TYROSINE KINASE (VEGFR-3),” describes materials and methods for screening for VEGFR-3 lymphedemas, and is incorporated herein by reference in its entirety. Where the lymphedema is a result of a mutation in Foxc2, the subject likely has LD.

Genetic diagnosis of a Foxc2 mutation (or a VEGFR-3 mutation or a Foxc1 mutation) can be performed using any available means for assaying DNA for a mutation. The assaying step may involve any techniques available for analyzing nucleic acid to determine its characteristics, including but not limited to well-known techniques such as single-strand conformation polymorphism analysis (SSCP) [Orita et al., Proc Natl. Acad. Sci. USA, 86: 2766-2770 (1989)]; heteroduplex analysis [White et al., Genomics, 12: 301-306 (1992)]; denaturing gradient gel electrophoresis analysis [Fischer et al., Proc. Natl. Acad. Sci. USA, 80: 1579-1583 (1983); and Riesner et al., Electrophoresis, 10: 377-389 (1989)]; DNA sequencing; RNase cleavage [Myers et al., Science, 230: 1242-1246 (1985)]; chemical cleavage of mismatch techniques [Rowley et al., Genomics, 30: 574-582 (1995); and Roberts et al., Nucl. Acids Res., 25: 3377-3378 (1997)]; restriction fragment length polymorphism analysis; single nucleotide primer extension analysis [Shumaker et al., Hum. Mutat., 7: 346-354 (1996); and Pastinen et al., Genome Res., 7: 606-614 (1997)]; 5′ nuclease assays [Pease et al., Proc. Natl. Acad. Sci. USA, 91:5022-5026 (1994)]; DNA Microchip analysis [Ramsay, G., Nature Biotechnology, 16: 40-48 (1999); and Chee et al., U.S. Pat. No. 5,837,832]; and ligase chain reaction [Whiteley et al., U.S. Pat. No. 5,521,065]. [See generally, Schafer and Hawkins, Nature Biotechnology, 16: 33-39 (1998).] All of the foregoing documents are hereby incorporated by reference in their entirety.

In one preferred embodiment, the assaying involves sequencing of nucleic acid to determine nucleotide sequence thereof, using any available sequencing technique. [See, e.g., Sanger et al., Proc. Natl. Acad. Sci. (USA), 74: 5463-5467 (1977) (dideoxy chain termination method); Mirzabekov, TIBTECH, 12: 27-32 (1994) (sequencing by hybridization); Drmanac et al., Nature Biotechnology, 16: 54-58 (1998); U.S. Pat. No. 5,202,231; and Science, 260: 1649-1652 (1993) (sequencing by hybridization); Kieleczawa et al., Science, 258: 1787-1791 (1992) (sequencing by primer walking); (Douglas et al., Biotechniques, 14: 824-828 (1993) (Direct sequencing of PCR products); and Akane et al., Biotechniques 16: 238-241 (1994); Maxam and Gilbert, Meth. Enzymol., 65: 499-560 (1977) (chemical termination sequencing), all incorporated herein by reference.] The analysis may entail sequencing of the entire Foxc2 gene genomic DNA sequence, or portions thereof; or sequencing of the entire Foxc2 coding sequence or portions thereof. In some circumstances, the analysis may involve a determination of whether an individual possesses a particular Foxc2 allelic variant, in which case sequencing of only a small portion of nucleic acid—enough to determine the sequence of a particular codon or codons characterizing the allelic variant—is sufficient. This approach is appropriate, for example, when assaying to determine whether one family member inherited the same allelic variant that has been previously characterized for another family member, or, more generally, whether a person's genome contains an allelic variant that has been previously characterized and correlated with a heritable lymphedema. More generally, the sequencing may be focused on those portions of the Foxc2 gene (or FoxC1 gene) that alter or eliminate the DNA binding domain or affect the promoter, or VEGFR-3 sequence that encode a VEGFR-3 kinase domain, since several different and apparently causative mutations in affected individuals that have been identified correspond to these types of mutations in Foxc2 and VEGFR-3.

In another embodiment, the assaying comprises performing a hybridization assay to determine whether nucleic acid from the human subject has a nucleotide sequence identical to or different from one or more reference sequences. In a preferred embodiment, the hybridization involves a determination of whether nucleic acid derived from the human subject will hybridize with one or more oligonucleotides, wherein the oligonucleotides have nucleotide sequences that correspond identically to a portion of the Foxc2 gene sequence, preferably the Foxc2 coding sequence set forth herein, or that correspond identically except for one mismatch, insertion, or deletion. The hybridization conditions are selected to differentiate between perfect sequence complementarity and imperfect matches differing by one or more bases. Such hybridization experiments thereby can provide single nucleotide polymorphism sequence information about the nucleic acid from the human subject, by virtue of knowing the sequences of the oligonucleotides used in the experiments.

Several of the techniques outlined above involve an analysis wherein one performs a polynucleotide migration assay, e.g., on a polyacrylamide electrophoresis gel, under denaturing or non-denaturing conditions. Nucleic acid derived from the human subject is subjected to gel electrophoresis, usually adjacent to one or more reference nucleic acids, such as reference Foxc2 sequences having a coding sequence identical to all or a portion of the Foxc2 sequences provided herein or another reported Foxc2 sequence, or identical except for one known polymorphism. The nucleic acid from the human subject and the reference sequence(s) are subjected to similar chemical or enzymatic treatments and then electrophoresed under conditions whereby the polynucleotides will show a differential migration pattern, unless they contain identical sequences. [See generally Ausubel et al. (eds.), Current Protocols in Molecular Biology, New York: John Wiley & Sons, Inc. (1987-1999); and Sambrook et al., (eds.), Molecular Cloning, A Laboratory Manual, Cold Spring Harbor, N.Y.: Cold Spring Harbor Laboratory Press (1989), both incorporated herein by reference in their entirety.]

In the context of assaying, the term “nucleic acid of a human subject” is intended to include nucleic acid obtained directly from the human subject (e.g., DNA or RNA obtained from a biological sample such as a blood, tissue, or other cell or fluid sample); and also nucleic acid derived from nucleic acid obtained directly from the human subject. By way of non-limiting examples, well known procedures exist for creating cDNA that is complementary to RNA derived from a biological sample from a human subject, and for amplifying (e.g., via polymerase chain reaction (PCR)) DNA or RNA derived from a biological sample obtained from a human subject. Any such derived polynucleotide which retains relevant nucleotide sequence information of the human subject's own DNA/RNA is intended to fall within the definition of “nucleic acid of a human subject” for the purposes of the present invention.

In the context of assaying, the term “mutation” includes addition, deletion, and/or substitution of one or more nucleotides in the Foxc2 gene sequence that are indicative of LD, or alternatively the VEGFR-3 gene sequence, which is indicative of lymphedema with different pathology and potentially different treatment regimens (or FoxC1). As reported herein, several Foxc2 mutations have been reported that play apparent causative roles in LD. Even mutations that have no apparent causative role may serve as useful markers for heritable lymphedema, provided that the appearance of the mutation correlates reliably with the appearance of lymphedema.

In such diagnostic methods, the polynucleotide sequences encoding FOXC2 or VEGFR3 may be used in hybridization or PCR assays of fluids or tissues from biopsies to detect expression of the appropriate protein. Such methods may be qualitative or quantitative in nature and may include Southern or northern analysis, dot blot or other membrane-based technologies; PCR technologies; dip stick, pin, chip and ELISA technologies. All of these techniques are well known in the art and are the basis of many commercially available diagnostic kits.

In order to conduct genetic analyses biological samples are obtained from the subject of interest. Any tissue or fluid sample that contains DNA is suitable, such as a tissue biopsy or blood sample. For some types of analysis, DNA from these samples is isolated using techniques well known to those of skill in the art. For example, DNA may be isolated from the EDTA-anticoagulated whole blood by the method of Miller et al., Nucleic Acids Res., 16: 1215 (1998), and from cytobrush specimens using the Puregene DNA isolation kit (Gentra Systems, Minneapolis, Minn.).

PCR as described in U.S. Pat. Nos. 4,683,195 and 4,965,188 provides additional uses for oligonucleotides based upon the FOXC2 or other sequence being tested. Such oligomers are generally chemically synthesized, but they may be generated enzymatically or produced from a recombinant source as described herein above. Oligomers generally comprise two nucleotide sequences, one with sense orientation and one with antisense, employed under optimized conditions for identification of a specific gene or condition. The same two oligomers, nested sets of oligomers, or even a degenerate pool of oligomers may be employed under less stringent conditions for detection and/or quantitation of closely related DNA or RNA sequences.

Using such oligomers, the sequence of the gene being tested for a mutation is screened for variation by direct sequencing of portions of gene. The sequencing strategy uses amplification primers generated based upon the cDNA sequence of the gene of interest (i.e., Foxc2 or VEGFR-3). Preferably, the primers are designed to amplify a region of interest known to have a mutation. Amplification and sequencing primers may be readily synthesized using techniques well known to those of skill in the art. Amplification primers also may be tagged at the 5′ end with the forward or reverse M13 universal sequence to facilitate direct sequencing. Amplimers were subjected to cycle sequencing using the dRhodamine terminator ready reaction kit or the Dye Primer ready reaction kit for −M13 and M13 Rev primers (Perkin Elmer) and analyzed on the Prism ABI 377 fluorescent sequencer. Sequences can then be aligned for further analysis using a program such as, e.g., SEQUENCHER 3.0 (Gene Codes).

Screening for FoxC2 mutation also is contemplated as a method for identifying subjects having, or at increased risk for developing, CVI.

B. Other Molecular Diagnoses

The work described herein identifies certain aberrant genetic expression patterns in LD patients and Foxc2 knockout mice, including elevated expression of at least one PDGF molecule, PDGF-B, in the lymphatics of LD patients. Elevated expression of at least one component of the blood vessel basal lamina, collagen IV, also was observed in lymphatic vessels with LD. These observations can be used as another tool for diagnosis LD in particular, as well as diagnosing other arterializations of lymphatic tissue that would benefit from materials and methods of treatment of this invention. Numerous well known techniques exist for quantifying gene expression, including but not limited to Northern hyrbridizations and in situ hybridizations with oligonucleotide probes; quantitative PCR; and Western blotting and in situ studies using antibodies for lymphatic and blood marker antigens and antigens of interest, such as PDGF's and PDGFR's and PC/SMC.

Methods to quantify the expression of a particular molecule include radiolabeling (Melby et al., J Immunol Methods 159: 235-44, 1993) or biotinylating (Duplaa et al., Anal Biochem 229-36, 1993) nucleotides, co-amplification of a control nucleic acid, and standard curves onto which the experimental results are interpolated.

Moreover, monitoring expression patterns of FOXC2, PDGFs (PDGF-A, -B, -C, or D), PDGFRs (alpha or beta), or collagen IV is informative for evaluating the efficacy of a particular therapeutic treatment regime in animal studies, in clinical trials, or in monitoring the treatment of an individual patient. In order to provide a basis for the diagnosis of disease, an expression profile for these genes in healthy and LD subjects can be established. This generally involves a combination of body fluids or cell extracts taken from normal subjects, either animal or human, with FOXC2, or a portion thereof, under conditions suitable for hybridization or amplification. Standard hybridization may be quantified by comparing the values obtained for normal subjects with a dilution series of FOXC2 run in the same experiment where a known amount of purified FOXC2 is used. Standard values obtained from normal samples may be compared with values obtained from samples from subjects thought to be afflicted with a FOXC2 caused lymphedema. Deviation between standard and subject values establishes the presence of disease.

C. Physical and Pathological Diagnosis

Lymphedema manifests itself with a number of physical symptoms that are familiar to medical practitioners and that are useful in addition to (or in alternative to) genetic and molecular approaches for diagnosis described herein. In other words, physical examinations and medical and family histories of a subject may be used to determine the presence of a lymphedema phenotype. For example, a few symptoms of hereditary lymphedema include asymmetry or obvious swelling of one or both legs, family reports of extremity swelling or asymmetry, mild or intermittent swelling, heavyset legs, obesity, or a history of leg infections.

Distichiasis (a double row of eyelashes) represents another diagnsostic criteria for LD.

Pathological examination, e.g., of tissue biopsies from a subject, may be evaluated as part of a lymphedema diagnsosis. For example, a skin biopsy is assessed for the presence of tortured lymphatic vessels and/or the presence of SMCs/PCs associated with lymphatic through appropriate microscopy techniques. Markers for identifying lymphatic endothelia, SMCs/PCs, and other cell types are described herein and known in the art, as are immunohistochemistry and fluorescence microlymphangiography techniques. (A) Arterial endothelial cell markers include ephrinB2, notch1-notch4, jagged1, D111, D114, transcription factors Hey1 and Hey2, hyaluronan receptor CD44, and neuropilin-1. Venous endothelial cell markers include EphB4 and neuropilin-2. Lymphatic endothelial markers include VEGFR-3 and LYVE-1, podoplanin and PROX1. In such techniques, lymphatic capillaries are made visible by fluorescence microlymphangiography upon fixation in a microscope slide. Standard hematoxylin and eosin staining of a thin section can be used to show the presence of the vessels and surrounding tissue.

Computed tomography (CT) may be used to show the presence of edema. X-ray lymphography, which involves the injection of a substance into the lymphatic vessels that shows up on X-rays—also may be performed to visualize the lymphatic vessels and/or any blockage associated with lymphedema. Other techniques that are well known include assessing the capillary density. This may be assessed by determining the presence of capillaries in the skin using simultaneous dual-site fluorescence angiography (fluorescence microlymphography FML). In such a procedure, the test patient is placed in a prone position with its arms extended out to the side and a microscope is positioned over each arm. This allows the practitioner to view the skin and record the magnified image on videotape for later analysis. A fluorescent dye (e.g., sodium fluorescein) is injected intravenously. Once the dye is injected, it circulates in the blood stream and enters the skin capillaries. Through the microscope, the capillaries appear as fluorescent dots, so that a count of the number of capillaries can be made in a standard area. While viewing the forearm with the microscope, a dye e.g., FITC-dextran may be injected into the skin. This fluorescent molecule is taken up by the lymphatic vessels which appear clearly as white lines spreading from the dye depot. In the swollen arm, lymphatic density is increased compared with the nonswollen arm, as was the spread of dye within the lymphatics horizontally along the skin.

The diameter of lymphatic vessels also may be determined in the skin by using FML as described above. Computer analysis may be used to analyse images generated by FML. In lymphedema cases, the lymphatic vessels may be constricted as compared to control vessels. The abnormal accumulation of SMCs/PCs around the lymphatic vessel may cause such constriction.

The simple diagnostic tool of isotope lymphography (lymphoscintigraphy or lymphangioscintigraphy) also may be used as a diagnostic test. In most centers where specialists in nuclear medicine are available, lymphoscintigraphy, has largely replaced conventional oil contrast lymphography for visualization of peripheral lymphatics. Although lymphoscintigraphy may use a variety of different tracers, different injected volumes and radioactivity, intracutaneous versus subcutaneous injection, epi- or subfascial injection, one or more injections, carried out at rest or under different protocols of physical activity, varied imaging times. This technique has gained popularity because the results are easily repeated for verification, offer remarkable insight into the underlying anatomic and functional lymphatic derangement.

Lymphoscintigraphy provides both images of lymphatics and lymph nodes as well as quantitative data on tracer (lymph) transport. Since its introduction, dermal injections of blue-dye are rarely indicated, however, such FML techniques may still be used. Direct oil contrast lymphography, also may be used. Non-invasive duplex-Doppler studies and on occasion phlebography may be appropriate in selected patients to examine the deep venous system and supplement the clinical impression of peripheral lymphedema. Other diagnostic and investigative tools of lymphologists: and lymphological centers include magnetic resonance imaging, computed tomography, ultrasonography, indirect (water soluble) lymphography and fluorescent microlymphangiography.

In other circumstances, lymph node biopsy may be used using care to remove regional lymph nodes using fine needle aspiration biopsy with cytological examination by a skilled pathologist. This may be a particularly useful diagnostic method in circumstances where malignancy is suspected.

In a similar fashion, physicians are familiar with CVI and are capable of diagnosing this condition (as well as varicose veins or hemorrhoids) from a physical examination. Doppler bidirectional flow studies and Doppler color-flow studies may be used to assess venous flow, its direction, and the presence of thrombi. Patient reports of leg discomfort and nonhealing ulcers are relevant to diagnosis. Absence of venous valves is considered causative of CVI.

III. Therapeutic and Prophylactic Treatment for Lymphedema and Lymphatic Arterialization and CVI

A variety of new approaches for therapeutic intervention are described herein. For subjects with impaired lymphatic function, e.g., due to lymphedema distichiasis or other condition causing arterialization of lymphatic endothelia, the therapy is intended to ameliorate at least one of the patient's symptoms. For example, therapy is intended to improve lymphatic function, reduce edema, reduce pain, reducing swelling, reduce incidences of infection, and/or reduce any other symptoms or complications of lymphedema. In preferred embodiments, the therapy also stops progression of, and preferably reverses, pathological manifestations of the lymphedema/arterialization, such as reducing the investment of SMC/PC's in lymphatic vessels and/or causing growth of new lymphatic vessels substantially free of the SMC/PC.

In one approach, a therapeutic is delivered to inhibit recruitment of SMC/PC to lymphatic vessels. In one variation, inhibitors of PDGF recruitment of SMC/PC that express PDGF receptors is targeted. In a preferred variation, inhibitors of PDGFR-beta activation (through stimulation by PDGF-B or PDGF-D ligands, or PDGF-A or -C stimulation of PDGFR-alpha/beta heterodimers) is contemplated. In a highly preferred embodiment, inhibition of PDGFR-beta activity due to PDGF-B stimulation is targeted.

In another approach, a therapeutic is delivered to ameliorate the effects of SMC/PC on the lymphatic vessels. Administration of smooth muscle cell inhibitors is specifically contemplated.

In another approach, an agent that causes increased FOXC2 expression is contemplated. For example, TGF-beta induces FOXC2 expression and TGF ligands represent another therapeutic approach for LD.

The following sections further describe exemplary therapeutic agents and methods of formulating and delivering such agents.

A. Polypeptide “Ligand Traps”

Polypeptides that can bind to circulating PDGF growth factors, especially PDGF-B, and inhibit the growth factor from binding to or stimulating PDGF receptors on cell surfaces are effective for inhibiting PDGF activation of PDGF receptors.

1. PDGFR-Alpha-Derived PDGF Ligand Traps

In one embodiment, a therapeutic agent comprises a polypeptide similar or identical in amino acid sequence to a PDGFR-α polypeptide or fragment thereof, preferably from the same species as the subject to be treated. Thus, for human treatment, a preferred polypeptide comprises an amino acid similar or identical to a fragment human PDGFR-alpha (e.g., SEQ ID NO: 14), where the fragment and the polypeptide bind one or more growth factors selected from the group consisting of PDGF-A, PDGF-B, and PDGF-C. The fragment minimally comprises enough of the PDGFR-α sequence to bind the ligand, and may comprise the complete receptor. Soluble extracellular domain fragments are preferred. Preferred polypeptides have an amino acid sequence at least 80% identical to a ligand binding fragment of human PDGFR-alpha. Fragments that are more similar, e.g., 85%, 90%, 91%, 92%, 93%, 94%, 95%, 96%, 97%, 98%, 99%, 99.5%, or 100% are highly preferred. Fragments that are 35%, 40%, 45%, 50%, 55%, 60%, 65%, 70%, and 75% are also contemplated. A genus of similar polypeptides can alternatively be defined by the ability of encoding polynucleotides to hybridize to the complement of a nucleotide sequence that corresponds to the cDNA sequence encoding the R-α receptor.

Preferred polypeptides may also be described as having an amino acid sequence encoded by a nucleic acid sequence at least 80% identical to a fragment of SEQ ID NO:13 encoding a ligand binding fragment of PDGFR-α. Nucleic acid fragments that are more similar, e.g., 85%, 90%, 91%, 92%, 93%, 94%, 95%, 96%, 97%, 98%, 99%, 99.5%, or 100% are highly preferred. Fragments that are 35%, 40%, 45%, 50%, 55%, 60%, 65%, 70%, and 75% are also contemplated. For example, a preferred binding unit polypeptide comprises an amino acid sequence that binds one or more PDGFR-α ligands and that is encoded by a nucleotide sequence that hybridizes to the complement of SEQ ID NO: 13 under moderately or highly stringent conditions. The term “highly stringent conditions” refers to hybridization/wash conditions selected to only permit hybridization of DNA strands whose sequences are highly complementary, and to exclude hybridization of significantly mismatched DNAs. Hybridization stringency is principally determined by temperature, ionic strength, and the concentration of denaturing agents such as formamide. Examples of “highly stringent conditions” for hybridization and washing are 0.015 M sodium chloride, 0.0015 M sodium citrate at 65-68° C. or 0.015 M sodium chloride, 0.0015 M sodium citrate, and 50% formamide at 42° C. See Sambrook, Fritsch & Maniatis, Molecular Cloning: A Laboratory Manual, 2nd Ed., Cold Spring Harbor Laboratory, (Cold Spring Harbor, N.Y. 1989); and Anderson et al., Nucleic Acid Hybridization: a Practical approach, Ch. 4, IRL Press Limited (Oxford, England). Limited, Oxford, England. Other agents may be included in the hybridization and washing buffers for the purpose of reducing non-specific and/or background hybridization. Examples are 0.1% bovine serum albumin, 0.1% polyvinyl-pyrrolidone, 0.1% sodium pyrophosphate, 0.1% sodium dodecylsulfate (NaDodSO4 or SDS), ficoll, Denhardt's solution, sonicated salmon sperm DNA (or another non-complementary DNA), and dextran sulfate, although other suitable agents can also be used. The concentration and types of these additives can be changed without substantially affecting the stringency of the hybridization conditions. Hybridization experiments are usually carried out at pH 6.8-7.4, 6.8-7.4; however, at typical ionic strength conditions, the rate of hybridization is nearly independent of pH. See Anderson et al., Nucleic Acid Hybridization: a Practical Approach, Ch. 4, IRL Press Limited (Oxford, England).

Exemplary PDGFR-α fragments comprise an amino terminal residue selected from the group consisting of positions 1 to 123 of SEQ ID NO: 14, and a carboxy terminal residue selected from the group consisting of positions 313 to 524 of SEQ ID NO: 14, wherein the PDGFR-α fragment binds at least one of PDGF-A, PDGF-B, and PDGF-C.

Where a polypeptide differs in sequence from a fragment of the receptor found in the target species, the amino acid differences preferably are conserved substitutions. Conservative amino acids can be grouped as described in Lehninger, (Biochemistry, Second Edition; Worth Publishers, Inc. NY:NY, pp. 71-77 (1975)) as set out in the following:

Non-Polar (Hydrophobic)

A. Aliphatic: A, L, I, V, P,
B. Aromatic: F, W,
C. Sulfur-containing: M,
D. Borderline: G.

Uncharged-polar
A. Hydroxyl: S, T, Y,
B. Amides: N, Q,
C. Sulfhydryl: C,
D. Borderline: G.

Positively Charged (Basic): K, R, H.

Negatively Charged (Acidic): D, E.

2. PDGFR-Beta-Derived PDGF Ligand Traps

In one embodiment, a therapeutic agent for use in practicing the invention comprises a polypeptide similar or identical in amino acid sequence to a PDGFR-β polypeptide or fragment thereof, preferably from the species to be treated. Thus, for human subjects, a polypeptide that comprises an amino acid similar or identical to a fragment of human PDGFR-beta (e.g., SEQ ID NO: 16), where the fragment and the polypeptide binds one or more growth factors selected from the group consisting of PDGF-B and PDGF-D. The fragment minimally comprises enough of the PDGFR-β sequence to bind the ligand, and may comprise the complete receptor. Soluble extracellular domain fragments are preferred. Preferred polypeptides have an amino acid sequence at least 80% identical to a ligand binding fragment of PDGFR-beta. Fragments that are more similar, e.g., 85%, 90%, 91%, 92%, 93%, 94%, 95%, 96%, 97%, 98%, 99%, 99.5%, or 100% are highly preferred. Fragments that are 35%, 40%, 45%, 50%, 55%, 60%, 65%, 70%, and 75% are also contemplated.

A genus of PDGFR-like polypeptides can alternatively be defined by the ability of encoding polynucleotides to hybridize to the complement of a nucleotide sequence that corresponds to the cDNA sequence encoding the PDGFR-β receptor. Preferred polypeptides may be described as having an amino acid sequence encoded by a nucleic acid sequence at least 80% identical to a fragment of SEQ ID NO:15 encoding a ligand binding fragment of PDGFR-β. Nucleic acid fragments that are more similar, e.g., 85%, 90%, 91%, 92%, 93%, 94%, 95%, 96%, 97%, 98%, 99%, 99.5%, or 100% are highly preferred. Fragments that are 35%, 40%, 45%, 50%, 55%, 60%, 65%, 70%, and 75% are also contemplated. For example, a preferred polypeptide comprises an amino acid sequence that binds one or more PDGFR-β ligands and that is encoded by a nucleotide sequence that hybridizes to the complement of SEQ ID NO: 15 under moderately or highly stringent conditions.

Exemplary PDGFR-β fragments have an amino terminal residue selected from the group consisting of positions 1 to 124 of SEQ ID NO: 16, and a carboxy terminal residue selected from the group consisting of positions 314 to 531 of SEQ ID NO: 16, wherein PDGFR-β fragment binds at least one of PDGF-B and PDGF-D.

3. Making Polypeptide Therapeutics

Polypeptide therapeutics may be made recombinantly using well known recombinant expression and purification techniques, or made synthetically.

For recombinant expression, a polynucleotide comprising a nucleotide sequence encoding the polypeptide is inserted into an expression vector designed for expression in a host cell, such as a bacterial, yeast, plant, insect, mammalian, human, or other cell type. Numerous commercially available vectors exist for a variety of cell types. Such vectors typically include elements such as an origin of replication, a promoter, enhance elements, a ribosome binding site, a polyadenylation signal sequence, a transcription termination sequence, sequences encoding a selectable marker, and/or intron sequences.

Likewise, well known techniques are available for polypeptide purification, including but not limited to chromatographic techniques (HPLC, ion exchange, metal ion chelate, size exclusion), antibody affinity chromatography, molecular sieve chromatography, isoelectric focusin, ligand/receptor affinity chromatography, and so on.

4. Additional Substituents

Polypeptide, antibody, and other therapeutics described herein may be chemically modified with various substituents. Such modifications preferably do not substantially reduce the growth factor (or receptor) binding affinities or specificities of the therapeutic core molecule. Rather, the chemical modifications impart additional desirable characteristics as discussed herein. Chemical modifications may take a number of different forms such as heterologous peptides, polysaccarides, lipids, non-standard amino acid resides, and nucleic acids, and metal chelates.

Polypeptide and antibodies described herein may be fused to heterologous peptides to confer various properties, e.g., increased solubility, modulation of clearance, targeting to particular cell or tissue types. In some embodiments, the receptor fragment is linked to a Fc domain of IgG or other immunoglobulin to improve serum half-life. Making Fc fusion constructs is known in the art and described, e.g., in WO 02/060950.

For substituents such as an Fc region of human IgG, the fusion can be fused directly to a binding construct or fused through an intervening sequence. For example, a human IgG hinge, CH2 and CH3 region may be fused at either the N-terminus or C-terminus of a receptor fragment to attach the Fc region. The resulting Fc-fusion construct enables purification via a Protein A affinity column (Pierce, Rockford, Ill.). Peptide and proteins fused to an Fc region can exhibit a substantially greater half-life in vivo than the unfused counterpart. A fusion to an Fc region allows for dimerization/multimerization of the fusion polypeptide. The Fc region may be a naturally occurring Fc region, or may be modified for superior characteristics, e.g., therapeutic qualities, circulation time, reduced aggregation.

Polypeptides can be modified, for instance, by glycosylation, amidation, carboxylation, or phosphorylation, or by the creation of acid addition salts, amides, esters, in particular C-terminal esters, and N-acyl derivatives. The proteins also can be modified to create peptide derivatives by forming covalent or noncovalent complexes with other moieties. Covalently bound complexes can be prepared by linking the chemical moieties to functional groups on the side chains of amino acids comprising the peptides, or at the N- or C-terminus.

Cysteinyl residues most commonly are reacted with haloacetates (and corresponding amines), such as chloroacetic acid or chloroacetamide, to give carboxymethyl or carbocyamidomethyl derivatives. Cysteinyl residues also are derivatized by reaction with bromotrifluoroacetone, α-bromo-β(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, orchloro-7-nitrobenzo-2-oxa-1,3-diazole.

Histidyl residues are derivatized by reaction with diethylprocarbonate at pH 5.5-7.0 because this agent is relatively specific for the histidyl side chain. Para-bromophenacyl bromide also is useful; the reaction is preferably performed in 0.1M sodium cacodylate at pH 6.0.

Lysinyl and amino terminal residues are reacted with succinic or carboxylic acid anhydrides. Derivatization with these agents has the effect of reversing the charge of the lysinyl residues. Other suitable reagents for derivatizing α-amino-containing residues include imidoesters such as methyl picolinimidate; pyridoxal phosphate; pyridoxal; chloroborohydride; trinitrobenzenesulfonic acid; O-methylissurea; 2,4 pentanedione; and transaminase catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pK of the guanidine functional group. Furthermore, these reagents may react with the groups of lysine as well as the arginine epsilon-amino group.

The specific modification of tyrosyl residues per se has been studied extensively, with particular interest in introducing spectral labels into tyrosyl residues by reaction with aromatic diazonium compounds or tetranitromethane. Most commonly, N-acetylimidizol and tetranitromethane are used to form O-acetyl tyrosyl species and 3-nitro derivatives, respectively. Tyrosyl residues are iodinated using 125I or 131I to prepare labeled proteins for use in radioimmunoassay.

Carboxyl side groups (aspartyl or glutamyl) are selectively modified by reaction with carbodiimides (R1) such as 1-cyclohexyl-3-(2-morpholinyl-(4-ethyl) carbodiimide or 1-ethyl-3 (4 azonia 4,4-dimethylpentyl)carbodiimide. Furthermore, aspartyl and glutamyl residues are converted to asparaginyl and glutaminyl residues by reaction with ammonium ions.

Derivatization with bifunctional agents is useful for crosslinking the binding construct to water-insoluble support matrixes. Such derivation may also provide the linker that may connect adjacent binding elements in a binding construct, or a binding elements to a heterologous peptide, e.g., a Fc fragment. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiiobis(succinimidylpropioonate), and bifunctional maleimides such as bis-N-maleimido-1,8-octane. Derivatizing agents such as methyl-3-[(p-azidophenyl) dithio] propioimidate yield photoactivatable intermediates that are capable of forming cross links in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440, incorporated herein by reference, are employed for protein immobilization.

Glutaminyl and asparaginyl residues are frequently deamidated to the corresponding glutamyl and aspartyl residues. Alternatively, these residues are deamidated under mildly acidic conditions.

Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecule Properties, W. H. Freeman & Co., San Francisco, pp. 79-86, 1983), acetylation of the N-terminal amine, and, in some instances, amidation of the C-terminal carboxyl groups. Such derivatives are chemically modified polypeptide compositions in which the binding construct polypeptide is linked to a polymer. The polymer selected is typically water soluble so that the protein to which it is attached does not precipitate in an aqueous environment, such as a physiological environment. The polymer selected is usually modified to have a single reactive group, such as an active ester for acylation or an aldehyde for alkylation, so that the degree of polymerization may be controlled as provided for in the present methods. The polymer may be of any molecular weight, and may be branched or unbranched. Included within the scope of the binding construct polypeptide polymers is a mixture of polymers. Preferably, for therapeutic use of the end-product preparation, the polymer will be pharmaceutically acceptable.

The polymers each may be of any molecular weight and may be branched or unbranched. The polymers each typically have an average molecular weight of between about 2 kDa to about 100 kDa (the term “about” indicating that in preparations of a water soluble polymer, some molecules will weigh more, some less, than the stated molecular weight). The average molecular weight of each polymer is between about 5 kDa and about 50 kDa, more preferably between about 12 kDa to about 40 kDa and most preferably between about 20 kDa to about 35 kDa.

Suitable water soluble polymers or mixtures thereof include, but are not limited to, N-linked or O-linked carbohydrates, sugars, phosphates, carbohydrates; sugars; phosphates; polyethylene glycol (PEG) (including the forms of PEG that have been used to derivatize proteins, including mono-(C1-C10) alkoxy- or aryloxy-polyethylene glycol); monomethoxy-polyethylene glycol; dextran (such as low molecular weight dextran, of, for example about 6 kD), cellulose; cellulose; other carbohydrate-based polymers, poly-(N-vinyl pyrrolidone)polyethylene glycol, propylene glycol homopolymers, a polypropylene oxide/ethylene oxide co-polymer, polyoxyethylated polyols (e.g., glycerol) and polyvinyl alcohol. Also encompassed by the present invention are bifunctional crosslinking molecules which may be used to prepare covalently attached multimers.

In general, chemical derivatization may be performed under any suitable condition used to react a protein with an activated polymer molecule. Methods for preparing chemical derivatives of polypeptides will generally comprise the steps of (a) reacting the polypeptide with the activated polymer molecule (such as a reactive ester or aldehyde derivative of the polymer molecule) under conditions whereby the binding construct becomes attached to one or more polymer molecules, and (b) obtaining the reaction product(s). The optimal reaction conditions will be determined based on known parameters and the desired result. For example, the larger the ratio of polymer molecules:protein, the greater the amount of attached polymer molecule. In one embodiment, the binding construct polypeptide derivative may have a single polymer molecule moiety at the amino terminus. (See, e.g., U.S. Pat. No. 5,234,784).

A particularly preferred water-soluble polymer for use herein is polyethylene glycol (PEG). As used herein, polyethylene glycol is meant to encompass any of the forms of PEG that can be used to derivatize other proteins, such as mono-(C1-C10) alkoxy- or aryloxy-polyethylene glycol. PEG is a linear or branched neutral polyether, available in a broad range of molecular weights, and is soluble in water and most organic solvents. PEG is effective at excluding other polymers or peptides when present in water, primarily through its high dynamic chain mobility and hydrophibic nature, thus creating a water shell or hydration sphere when attached to other proteins or polymer surfaces. PEG is nontoxic, non-immunogenic, and approved by the Food and Drug Administration for internal consumption.

Proteins or enzymes when conjugated to PEG have demonstrated bioactivity, non-antigenic properties, and decreased clearance rates when administered in animals. F. M. Veronese et al., Preparation and Properties of Monomethoxypoly(ethylene glycol)-modified Enzymes for Therapeutic Applications, in J. M. Harris ed., Poly(Ethylene Glycol) Chemistry—Biotechnical and Biomedical Applications, 127-36, 1992, incorporated herein by reference. These phenomena are due to the exclusion properties of PEG in preventing recognition by the immune system. In addition, PEG has been widely used in surface modification procedures to decrease protein adsorption and improve blood compatibility. S. W. Kim et al., Ann. N.Y. Acad. Sci. 516: 116-30 1987; Jacobs et al., Artif. Organs 12: 500-501, 1988; Park et al., J. Poly. Sci, Part A 29:1725-31, 1991, incorporated herein by reference. Hydrophobic polymer surfaces, such as polyurethanes and polystyrene can be modified by the grafting of PEG (MW 3,400) and employed as nonthrombogenic surfaces. Surface properties (contact angle) can be more consistent with hydrophilic surfaces, due to the hydrating effect of PEG. More importantly, protein (albumin and other plasma proteins) adsorption can be greatly reduced, resulting from the high chain motility, hydration sphere, and protein exclusion properties of PEG.

PEG (MW 3,400) was determined as an optimal size in surface immobilization studies, Park et al., J. Biomed. Mat. Res. 26:739-45, 1992, while PEG (MW 5,000) was most beneficial in decreasing protein antigenicity. (F. M. Veronese et al., In J. M. Harris, et al., Poly(Ethylene Glycol) Chemistry—Biotechnical and Biomedical Applications, 127-36.)

Methods for preparing pegylated binding construct polypeptides will generally comprise the steps of (a) reacting the polypeptide with polyethylene glycol (such as a reactive ester or aldehyde derivative of PEG) under conditions whereby the binding construct polypeptide becomes attached to one or more PEG groups, and (b) obtaining the reaction product(s). In general, the optimal reaction conditions for the acylation reactions will be determined based on known parameters and the desired result. For example, the larger the ratio of PEG: protein, the greater the percentage of poly-pegylated product. In some embodiments, the binding construct will have a single PEG moiety at the N-terminus. See U.S. Pat. No. 5,234,784, herein incorporated by reference.

B. Nucleic Acid Therapeutics.

It is now widely recognized that DNA may be introduced into a cell using a variety of viral vectors in order to achieve expression of the DNA. This invention includes DNA/RNA-based therapeutics for expression of polypeptide or antibody active agents in vivo. Moreover, the invention includes DNA/RNA based therapeutics for expression of nucleic acid active agents in vivo, such as antisense, interfering RNA, apatamer, and ribozyme agents. Still another class of nucleic acid based therapies are nucleic acids (antisense, interfering RNA, aptamer, and ribozyme) delivered as therapeutics in their own right, with no need for in vivo transcription or translation.

Nucleic acid molecules that encode all or part of a polypeptide therapeutic, as well as nucleic acid therapeutics can be made using a variety of well known recombinant and synthetic techniques, including, without limitation, chemical synthesis, cDNA or genomic library screening, expression library screening, and/or PCR amplification of cDNA or genomic DNA. These methods and others useful for isolating such DNA are set forth, for example, by Sambrook, et al., “Molecular Cloning: A Laboratory Manual,” Cold Spring Harbor Laboratory Press, Cold Spring Harbor, N.Y. (1989), by Ausubel, et al., eds., “Current Protocols In Molecular Biology,” Current Protocols Press (1994), and by Berger and Kimmel, “Methods In Enzymology: Guide To Molecular Cloning Techniques,” vol. 152, Academic Press, Inc., San Diego, Calif. (1987). Preferred nucleic acid sequences are mammalian sequences, such as human, rat, and mouse.

Chemical synthesis of nucleic acid molecules can be accomplished using methods well known in the art, such as those set forth by Engels, et al., Angew. Chem. Intl. Ed., 28:716-734 (1989). These methods include, inter alia, the phosphotriester, phosphoramidite and H-phosphonate methods of nucleic acid synthesis. Nucleic acids larger than about 100 nucleotides in length can be synthesized as several fragments, each fragment being up to about 100 nucleotides in length. The fragments can then be ligated together, as described below, to form the full length nucleic acid of interest. A preferred method is polymer-supported synthesis using standard phosphoramidite chemistry.

1. Gene Delivery Vectors

In embodiments requiring polynucleotide expression, expression constructs comprising viral vectors containing the genes of interest may be replication-deficient retroviral vectors, including but not limited to lentivirus vectors [Kim et al., J. Virol., 72(1): 811-816 (1998); Kingsman & Johnson, Scrip Magazine, October, 1998, pp. 43-46.]; adenoviral (see for example, U.S. Pat. No. 5,824,544; U.S. Pat. No. 5,707,618; U.S. Pat. No. 5,693,509; U.S. Pat. No. 5,670,488; U.S. Pat. No. 5,585,362; each incorporated herein by reference), retroviral (see for example, U.S. Pat. No. 5,888,502; U.S. Pat. No. 5,830,725; U.S. Pat. No. 5,770,414; U.S. Pat. No. 5,686,278; U.S. Pat. No. 4,861,719 each incorporated herein by reference), adeno-associated viral (see for example, U.S. Pat. No. 5,474,935; U.S. Pat. No. 5,139,941; U.S. Pat. No. 5,622,856; U.S. Pat. No. 5,658,776; U.S. Pat. No. 5,773,289; U.S. Pat. No. 5,789,390; U.S. Pat. No. 5,834,441; U.S. Pat. No. 5,863,541; U.S. Pat. No. 5,851,521; U.S. Pat. No. 5,252,479 each incorporated herein by reference), an adenoviral-adenoassociated viral hybrid (see for example, U.S. Pat. No. 5,856,152 incorporated herein by reference) or a vaccinia viral or a herpesviral (see for example, U.S. Pat. No. 5,879,934; U.S. Pat. No. 5,849,571; U.S. Pat. No. 5,830,727; U.S. Pat. No. 5,661,033; U.S. Pat. No. 5,328,688 each incorporated herein by reference) vector. Replication-deficient viral vectors are preferred, such as lentivirus vectors [Kim et al., J. Virol., 72(1): 811-816 (1998); Kingsman & Johnson, Scrip Magazine, October, 1998, pp. 43-46.].

There are numerous published reports describing how to optimize gene delivery for in vivo and ex vivo situations. For viral vectors, one generally will prepare a viral vector stock. Depending on the kind of virus and the titer attainable, one will deliver 1×10⁴, 1×10⁵, 1×10⁶, 1×10⁷, 1×10⁸, 1×10⁹, 1×10¹⁰, 1×10¹¹ or 1×10¹² infectious particles to the patient. Similar figures may be extrapolated for liposomal or other non-viral formulations by comparing relative uptake efficiencies. Formulation as a pharmaceutically acceptable composition is discussed below.

Various routes are contemplated for delivery. For example, systemic delivery is contemplated, e.g., through intravenous or other injections. Intradermal and topical applications also are contemplated.

2. Antisense Nucleic Acids

Another approach for inhibiting SMC/PC association with lymphatic vessels is to inhibit expression of the secreted or cell surface proteins that promote the association, such as PDGFs, PDGF receptors. Antisense therapy is one method for inhibiting expression, described below with particular reference to PDGF-B and PDGFR-beta, with the understanding that the description is equally suitable for other gene targets identified herein.

In one embodiment, the invention provides methods using antisense oligonucleotides which negatively regulate PDGF-B or PDGFR-β expression via hybridization to messenger RNA (mRNA) encoding PDGF-B or PDGFR-β. PDGF-B and PDGFR-β is known to those of skill in the art e.g., those of skill in the art are referred NM_{—}033016; NM_{—}002608; NM_{—}011057; NM_{—}008809; and BC023427 for the sequence of PDGF-B; NM_{—}008809 shows the sequence of a mouse PDGFR-β polypeptide. These sequences may be used to prepare antisense molecules for one or both of PDGF or PDGFR β. Cohen-Sacks et al have described PDGFR-β antisense molecules encapsulated in polymeric nanospheres for the treatment of restenosis (Cohen-Sacks et al., Gene Ther. 2002 December; 9(23):1607-16). The results of those determinations showed that partially phosphorothioated antisense sequences were more specific than the fully phosphorothioated analogs. A significant antirestenotic effect of the naked antisense sequence and the antisense-NP (nanoparticles) was observed in the rat carotid in vivo model. It is contemplated that such molecules may be used in the methods of the present invention.

In one aspect, antisense oligonucleotides at least 5 to about 50 nucleotides in length, including all lengths (measured in number of nucleotides) in between, which specifically hybridize to mRNA encoding PDGF-B or PDGFR-β and inhibit mRNA expression, and as a result PDGF-B or PDGFR-β protein expression, are contemplated by the invention. Antisense oligonucleotides include those comprising modified internucleotide linkages and/or those comprising modified nucleotides which are known in the art to improve stability of the oligonucleotide, i.e., make the oligonucleotide more resistant to nuclease degradation, particularly in vivo. It is understood in the art that, while antisense oligonucleotides that are perfectly complementary to a region in the target polynucleotide possess the highest degree of specific inhibition, antisense oligonucleotides which are not perfectly complementary, i.e., those which include a limited number of mismatches with respect to a region in the target polynucleotide, also retain high degrees of hybridization specificity and therefore inhibit expression of the target mRNA. Accordingly, the invention contemplate methods using antisense oligonucleotides that are perfectly complementary to a target region in a polynucleotide encoding PDGF-B or PDGFR-β, as well as methods that utilize antisense oligonucleotides that are not perfectly complementary, i.e., include mismatches, to a target region in the target polynucleotide to the extent that the mismatches do not preclude specific hybridization to the target region in the target polynucleotide. Preparation and use of antisense compounds is described in U.S. Pat. No. 6,277,981, the disclosure of which is incorporated herein by reference in its entirety.

3. Ribozyme Therapeutics

Another class of therapeutics for inhibiting expression of the target genes described herein are ribozymes.

Ribozyme inhibitors include a nucleotide region which specifically hybridizes to a target polynucleotide and an enzymatic moiety that digests the target polynucleotide. Specificity of ribozyme inhibition is related to the length the antisense region and the degree of complementarity of the antisense region to the target region in the target polynucleotide. The invention therefore contemplate ribozyme inhibitors of PDGF-B or PDGFR-β comprising antisense regions from 5 to about 50 nucleotides in length, including all nucleotide lengths in between, that are perfectly complementary, as well as antisense regions that include mismatches to the extent that the mismatches do not preclude specific hybridization to the target region in the target PDGF-B or PDGFR-β-encoding polynucleotide. Ribozymes useful in methods of the invention include those comprising modified internucleotide linkages and/or those comprising modified nucleotides which are known in the art to improve stability of the oligonucleotide, i.e., make the oligonucleotide more resistant to nuclease degradation, particularly in vivo, to the extent that the modifications do not alter the ability of the ribozyme to specifically hybridize to the target region or diminish enzymatic activity of the molecule. Because ribozymes are enzymatic, a single molecule is able to direct digestion of multiple target molecules thereby offering the advantage of being effective at lower concentrations than non-enzymatic antisense oligonucleotides. Preparation and use of ribozyme technology is described in U.S. Pat. Nos. 6,696,250, 6,410,224, 5,225,347, the disclosures of which are incorporated herein by reference in their entireties.

4. RNA Interference and Lasso Therapies

Another class of therapeutics for inhibiting expression (and therefore activity) of target genes/pathways described herein is interfering RNA technology, also known as RNA interference (RNAi) or short interfering RNA (siRNA).

Using the knowledge of the sequence of target genes such as PDGF-B or PDGFR-β, siRNA molecules are formed that interfere with the expression of the genes. SiRNA describes a technique by which post-transcriptional gene silencing (PTGS) is induced by the direct introduction of double stranded RNA (dsRNA: a mixture of both sense and antisense strands). (Fire et al., Nature 391:806-811, 1998). Current models of PTGS indicate that short stretches of interfering dsRNAs (21-23 nucleotides; siRNA also known as “guide RNAs”) mediate PTGS. siRNAs are apparently produced by cleavage of dsRNA introduced directly or via a transgene or virus. These siRNAs may be amplified by an RNA-dependent RNA polymerase (RdRP) and are incorporated into the RNA-induced silencing complex (RISC), guiding the complex to the homologous endogenous mRNA, where the complex cleaves the transcript. It is contemplated that RNAi may be used to disrupt the expression of a gene in a tissue-specific manner. By placing a gene fragment encoding the desired dsRNA behind an inducible or tissue-specific promoter, it should be possible to inactivate genes at a particular location within an organism or during a particular stage of development.

In one aspect, the invention provides double-stranded RNA (dsRNA) wherein one strand is complementary to a target region in a target PDGF-B or PDGFR-β-encoding polynucleotide. In general, dsRNA molecules of this type less than 30 nucleotides in length are referred to in the art as short interfering RNA (siRNA). The invention also contemplates, however, use of dsRNA molecules longer than 30 nucleotides in length, and in certain aspects of the invention, these longer dsRNA molecules can be about 30 nucleotides in length up to 200 nucleotides in length and longer, and including all length dsRNA molecules in between. As with other RNA inhibitors, complementarity of one strand in the dsRNA molecule can be a perfect match with the target region in the target polynucleotide, or may include mismatches to the extent that the mismatches do not preclude specific hybridization to the target region in the target PDGF-B or PDGFR-β-encoding polynucleotide. As with other RNA inhibition technologies, dsRNA molecules include those comprising modified internucleotide linkages and/or those comprising modified nucleotides which are known in the art to improve stability of the oligonucleotide, i.e., make the oligonucleotide more resistant to nuclease degradation, particularly in vivo. Preparation and use of RNAi compounds is described in U.S. Patent Application No. 20040023390, the disclosure of which is incorporated herein by reference in its entirety.

The invention further contemplates methods wherein inhibition of PDGF-B or PDGFR-β is effected using RNA lasso technology. Circular RNA lasso inhibitors are highly structured molecules that are inherently more resistant to degradation and therefore do not, in general, include or require modified internucleotide linkage or modified nucleotides. The circular lasso structure includes a region that is capable of hybridizing to a target region in a target polynucleotide, the hybridizing region in the lasso being of a length typical for other RNA inhibiting technologies. As with other RNA inhibiting technologies, the hybridizing region in the lasso may be a perfect match with the target region in the target polynucleotide, or may include mismatches to the extent that the mismatches do not preclude specific hybridization to the target region in the target PDGF-B or PDGFR-β-encoding polynucleotide. Because RNA lassos are circular and form tight topological linkage with the target region, inhibitors of this type are generally not displaced by helicase action unlike typical antisense oligonucleotides, and therefore can be utilized as dosages lower than typical antisense oligonucleotides. Preparation and use of RNA lassos is described in U.S. Pat. No. 6,369,038, the disclosure of which is incorporated herein by reference in its entirety.

Anti-sense RNA and DNA molecules, ribozymes, RNAi and triple helix molecules directed against PDGF-B or PDGFR-β can be prepared by any method known in the art for the synthesis of DNA and RNA molecules. These include techniques for chemically synthesizing oligodeoxyribonucleotides well known in the art including, but not limited to, solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably or transiently into cells.

5. Aptamer Therapeutics

Aptamers are another nucleic acid based method for interfering with the interaction of PDGF-B and PDGFR-β is the use of an aptamer. Aptamers are DNA or RNA molecules that have been selected from random pools based on their ability to bind other molecules. Aptamers have been selected which bind nucleic acid, proteins, small organic compounds, and even entire organisms. Methods and compositions for identifying and making aptamers are known to those of skill in the art and are described e.g., in U.S. Pat. No. 5,840,867 and U.S. Pat. No. 5,582,981 each incorporated herein by reference. Aptamers that bind PDGF-B are known to those of skill in the art and are specifically contemplated to be useful in the present therapeutic embodiments.

Recent advances in the field of combinatorial sciences have identified short polymer sequences with high affinity and specificity to a given target. For example, SELEX technology has been used to identify DNA and RNA aptamers with binding properties that rival mammalian antibodies, the field of immunology has generated and isolated antibodies or antibody fragments which bind to a myriad of compounds and phage display has been utilized to discover new peptide sequences with very favorable binding properties. Based on the success of these molecular evolution techniques, it is certain that molecules can be created which bind to any target molecule. A loop structure is often involved with providing the desired binding attributes as in the case of: aptamers which often utilize hairpin loops created from short regions without complimentary base pairing, naturally derived antibodies that utilize combinatorial arrangement of looped hyper-variable regions and new phage display libraries utilizing cyclic peptides that have shown improved results when compared to linear peptide phage display results. Thus, sufficient evidence has been generated to suggest that high affinity ligands can be created and identified by combinatorial molecular evolution techniques. For the present invention, molecular evolution techniques can be used to isolate binding constructs specific for ligands described herein. For more on aptamers, See generally, Gold, L., Singer, B., He, Y. Y., Brody. E., “Aptamers As Therapeutic And Diagnostic Agents,” J. Biotechnol. 74:5-13 (2000). Relevant techniques for generating aptamers may be found in U.S. Pat. No. 6,699,843, which is incorporated by reference in its entirety.

In some embodiments, the aptamer may be generated by preparing a library of nucleic acids; contacting the library of nucleic acids with a growth factor, wherein nucleic acids having greater binding affinity for the growth factor (relative to other library nucleic acids) are selected and amplified to yield a mixture of nucleic acids enriched for nucleic acids with relatively higher affinity and specificity for binding to the growth factor. The processes may be repeated, and the selected nucleic acids mutated and re-screened, whereby a growth factor aptamer is be identified.

C. Antibody Therapeutics

Some of the targets described herein for therapeutic intervention are ciruculating polypeptides, such as the PDGF's, and others such as PDGFR's are expressed on the cell surface. In each case, antibody substances are contemplated as a further class of therapeutic for inhibition. For example, antibodies to either PDGF-B or to the extracellular domain of PDGFR-beta are useful for inhibiting PDGF-B stimulation of PDGFR-beta. Using the known protein sequences of the targets, it is possible to generate antibody substances against them using known techniques.

Methods and compositions for making monoclonal antibodies against PDGF-B are described in U.S. Pat. No. 5,889,149, incorporated herein by reference in its entirety. U.S. Pat. No. 6,358,954 describes inhibitors of PDGFR that may be useful for inhibiting the activity of PDGF Receptor β. U.S. Pat. No. 5,817,310 describes PDGFR-β antibodies and antibody fragments that inhibit ligand binding.

1. Generating Antibodies in Animals and Cells

Polyclonal or monoclonal therapeutic antibodies useful in practicing this invention may be prepared in laboratory animals or by recombinant DNA techniques.

Polyclonal antibodies to a PDGF or PDGFR molecule or a fragment thereof containing the target amino acid sequence generally are raised in animals by multiple subcutaneous (sc) or intraperitoneal (ip) injections of the molecule or fragment in combination with an adjuvant such as Freund's adjuvant (complete or incomplete). To enhance immunogenicity, it may be useful to first conjugate the molecule or a fragment containing the target amino acid sequence of a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl, or R1N═C═NR, where R and R1 are different alkyl groups. Alternatively, immunogenic conjugates can be produced recombinantly as fusion proteins.

Animals are immunized by combining about 1 mg or about 1 microgram of antigen (for rabbits or mice, respectively) with about 3 volumes of Freund's complete adjuvant and injecting the solution intradermally at multiple sites. Approximately 7 to 14 days later, animals are bled and the serum is assayed for anti-PDGF/PDGFR titer. Animals are boosted with antigen repeatedly until the titer plateaus. Preferably, the animal is boosted with the same molecule or fragment thereof as was used for the initial immunization, but conjugated to a different protein and/or through a different cross-linking agent. In addition, aggregating agents such as alum are used in the injections to enhance the immune response.

Monoclonal antibodies may be prepared by recovering spleen cells from immunized animals and immortalizing the cells in conventional fashion, e.g. by fusion with myeloma cells. The clones are then screened for those expressing the desired antibody.

Preferably, antibodies for administration to humans, especially when prepared in a laboratory animal such as a mouse, will be “humanized”, or made chimeric, so as to be more compatible with the human immune system such that a human patient will not develop an immune response to the antibody. Even more preferably, human antibodies which can now be prepared using methods such as those described for example, in Lonberg, et al., Nature Genetics, 7:13-21 (1994) are preferred for therapeutic administration to patients. Fully human antibodies are highly preferred.

2. Humanization Of Monoclonal Antibodies

Any known protocol may be used to improve the efficacy of monoclonal antibodies as therapeutics in humans, by “humanizing” the monoclonal antibodies to improve their serum half-life and render them less immunogenic in human hosts (i.e., to prevent human antibody response.

The principles of humanization have been described in the literature and are facilitated by the modular arrangement of antibody proteins. To minimize the possibility of binding complement, a humanized antibody of the IgG4 isotype is preferred.

For example, a level of humanization is achieved by generating chimeric antibodies comprising the variable domains of non-human antibody proteins of interest, such as the anti-PDGF-B monoclonal antibodies described herein, with the constant domains of human antibody molecules. (See, e.g., Morrison and Oi, Adv. Immunol., 44:65-92 (1989).) The variable domains of PDGF neutralizing antibodies are cloned from the genomic DNA of a B-cell hybridoma or from cDNA generated from mRNA isolated from the hybridoma of interest. The V region gene fragments are linked to exons encoding human antibody constant domains, and the resultant construct is expressed in suitable mammalian host cells (e.g., myeloma or CHO cells).

To achieve an even greater levels of humanization, only those portions of the variable region gene fragments that encode antigen-binding complementarity determining regions (“CDR”) of the non-human monoclonal antibody genes are cloned into human antibody sequences. [See, e.g., Jones et al., Nature, 321:522-525 (1986); Riechmann et al., Nature, 332:323-327 (1988); Verhoeyen et al., Science, 239:1534-36 (1988); and Tempest et al., Bio/Technology, 9:266-71 (1991).] If necessary, the B-sheet framework of the human antibody surrounding the CDR3 regions also is modified to more closely mirror the three dimensional structure of the antigen-binding domain of the original monoclonal antibody. [(See Kettleborough et al., Protein Engin., 4:773-783 (1991); and Foote et al., J. Mol. Biol., 224:487-499 (1992).)]

In an alternative approach, the surface of a non-human monoclonal antibody of interest is humanized by altering selected surface residues of the non-human antibody, e.g., by site-directed mutagenesis, while retaining all of the interior and contacting residues of the non-human antibody. [See Padlan, Molecular Immunol., 28(4/5):489-98 (1991).]

3. Human Antibodies from Phage Display

Preparation of antibodies using recombinant DNA methods such as the phagemid display method, may be accomplished using commercially available kits, as for example, the Recombinant Phagemid Antibody System available from Pharmacia (Uppsala, Sweden), or the SurfZAP™ phage display system (Stratagene Inc., La Jolla, Calif.).

Human PDGF- or PDGFR-neutralizing antibodies are generated by phage display techniques such as those described in Aujame et al., Human Antibodies, 8(4):155-168 (1997); Hoogenboom, TIBTECH, 15:62-70 (1997); and Rader et al., Curr. Opin. Biotechnol., 8:503-508 (1997), all of which are incorporated by reference. For example, antibody variable regions in the form of Fab fragments or linked single chain Fv fragments are fused to the amino terminus of filamentous phage minor coat protein pIII. Expression of the fusion protein and incorporation thereof into the mature phage coat results in phage particles that present an antibody on their surface and contain the genetic material encoding the antibody. A phage library comprising such constructs is expressed in bacteria, and the library is panned (screened) for PDGF or PDGFR-specific phage-antibodies using labeled or immobilized antigen-probe.

4. Human Neutralizing Antibodies from Transgenic Mice

Human neutralizing antibodies are generated in transgenic mice essentially as described in Bruggemann and Neuberger, Immunol. Today, 17(8):391-97 (1996) and Bruggemann and Taussig, Curr. Opin. Biotechnol., 8:455-58 (1997). Transgenic mice carrying human V-gene segments in germline configuration and that express these transgenes in their lymphoid tissue are immunized with a PDGF or PDGFR composition using conventional immunization protocols. Hybridomas are generated using B cells from the immunized mice using conventional protocols and screened to identify hybridomas secreting anti-PDGF or PDGFR human antibodies.

5. Bispecific Antibodies and Polypeptides

Bi-specific antibodies that specifically bind to multiple targets, such as PDGFR-alpha and beta, or PDGF-B and PDGF-C or -D, also are contemplated for therapeutic use herein. Such antibodies are produced, isolated, and tested using any available procedures. See, e.g., Pluckthun & Pack, Immunotechnology, 3:83-105 (1997); Carter et al., J. Hematotherapy, 4: 463-470 (1995); Renner & Pfreundschuh, Immunological Reviews, 1995, No. 145, pp. 179-209; Pfreundschuh U.S. Pat. No. 5,643,759; Segal et al., J. Hematotherapy, 4: 377-382 (1995); Segal et al., Immunobiology, 185: 390-402 (1992); and Bolhuis et al., Cancer Immunol. Immunother., 34: 1-8 (1991), all of which are incorporated herein by reference in their entireties.

Bi-specific antibodies that may be employed in combination with the binding constructs of the invention include those described in the co-owned U.S. Provisional Patent Application No. 60/550,511 filed Mar. 5, 2004, directed to “Multivalent Antibody Materials And Methods For VEGF/PDGF Family Of Growth Factors,” U.S. Provisional Application No. 60/586,662 filed Jul. 9, 2004; U.S. and PCT application Nos. ______ and ______ filed Mar. 7, 2005 (Attorney Docket No.: 28967/39820B; “Multivalent Antibody Materials And Methods For VEGF/PDGF Family of Growth Factors”).

Bi-specific PDGF binding compounds that may be employed in the invention are described in co-owned U.S. Provisional Patent Application No. 60/550,907, filed Mar. 5, 2004, directed to “Growth Factor Binding Constructs Materials and Methods;” and U.S. and PCT application Nos. ______ and ______ filed Mar. 7, 2005 (Attorney Docket No. 28967/39700A; “Growth Factor Binding Constructs Materials and Methods)

6. Domain Antibodies and Antibody Fragments

A domain antibody comprises a functional binding unit of an antibody, and can correspond to the variable regions of either the heavy (VH) or light (VL) chains of antibodies. In a preferred embodiment these antibody segments are linked by a peptide bond (optionally with a peptide linker) to form a single polypeptide chain that can be expressed in a recombinant host cell. A domain antibody can have a molecular weight of approximately 13 kDa, or approximately one-tenth of a full antibody. Domain antibodies may be derived from full antibodies such as those described herein.

The well-known structure of antibodies permits assemblage of a wide variety of antibody fragments and polypeptides that comprising antigen binding portions of antibodies and that retain the antigen binding specificity of the antibody. All such antibody substances, including but not limited to including Fab, Fab′, F(ab′)₂, Fv, single chain antibodies (scFv), and polypeptide and other molecules comprising the same, are contemplated for practicing this invention.

D. Polypeptide Inhibitors of PDGF's

Any polypeptide that has been identified as an inhibitor of PDGF expression or activity is contemplated as a therapeutic of the invention, as are fragments or variants thereof that retain PDGF inhibitor effects. Exemplary descriptions of variants are set forth above with respect to ligand trap polypeptides, and repeated here by reference.

Polypeptides specifically contemplated include low density liporotein receptor-related protein 1 (LRP1, SEQ ID NOs: 31-32), which forms a complex with PDGFR-beta and inhibits its activity (See Boucher et al., Science, 300, 329-332, 2003, incorporated here by reference), as well as fragments and variants thereof that form the complex and inhibit the activity.

Also contemplated are VE-statin proteins (SEQ ID NOs: 27-28 (VE Statin 2, and SEQ ID NOs: 29-30, VE Statin 1) secreted proteins produced by endothelial cells that inhibit PDGF-BB induced smooth muscle cell migration as described in Soncin et al., EMBO J, 22, 5700-5711, 2003, incorporated here by rerference). Fragments and variants that inhibit PDGF-BB-induced smooth muscle cell migration also are contemplated.

PDGFR-α has been shown to antagonize PDGFR-β-induced SMC migration. See Yu, J., et al., Biochem. Biophys. Res. Commun. 282:697-700 (2001). Neutralizing antibodies against PDGF-AA enhance smooth muscle cell (SMC) migration (Palumbo, R., et al., Arterioscler. Thromb. Vasc. Biol. 22:405-11 (2002). These data provide a therapeutic indication for PDGFR-alpha ligands such as PDGF-AA homodimers.

E. Small Molecule Inhibitors

Any chemical substance that can be safely administered as a therapeutic and that can be used to modulate biochemical pathway targets identified herein, such as PDGF-medicated stimulation of PDGF receptors, may be used to practice the invention.

The PDGF receptors are receptor tyrosine kinases and intracellular signaling is initiated through receptor phosphorylation. Accordingly, one preferred class of molecules for practice of the invention is tyrosine kinase inhibitors, including those described in Morin, Oncogene, 19(56):6574-83, 2000, incorporated herein by reference.

Exemplary Platelet-Derived Growth Factor Receptor (PDGF-R) inhibitors include those described in the following table. All references cited therein are incorporated by reference in their entirety:

| | | Patent (U.S. Unless |
|---|---|---|
| Brand Name or | | Specified) or |
| Product # | Generic Name | Reference |
| Gleevec | imatinib mesylate | 5,521,184; Druker, |
| (Glivec) | (CGP57148B; STI-571) | Nature Medicine, |
| | | 2: 561 (1996) |
| AG1295 | | Kovalenko, Cancer |
| | | Res 54: 6106 |
| | | (1994) |
| AG1296 | | Kovalenko, Cancer |
| | | Res 54: 6106 |
| | | (1994) |
| | triazolopyrimidine | |
| | 2-phenylaminopyrimidine | |
| | class compounds | |
| 3744W | | Spacey et al., |
| | | Bioch. Pharm. |
| | | 55(3): 261-71, |
| | | 1998 Feb. 1 |
| Tyrphostin AG1296 | | Kovalenko et al, |
| | | Cancer Res. 1994 |
| | | Dec 1; 54(23) |
| CGP53716 | | Buchdunger et al., |
| | | PNAS 1995 |
| | | 92(7): 2558-62 |
| CGP57148 | | Buchdunger et al., |
| | | Cancer Res. 1996 |
| | | 56(1): 100-4 |
| CT52923 | | |
| RP-1776 | | |
| GFB-111 | | |
| | a pyrrolo[3,4-c]-beta- | |
| | carboline-dione | |
| SU11248 (SU01248) | | |
| PKC787 | | Novartis |
| PTK787 | | Novartis |
| DMBI | | Organon; Zaman, |
| | | Biochem. |
| | | Pharmacol. 57: 57 |
| | | 1999 |
| SU101 (LFM, | | Sugen; Shawner, |
| HWA486) | | Clin. Cancer. Res., |
| | | 3: 1167 1997 |
| SU0020 (A771726) | | Sugen |
| Comp. 54 | | Parke-Davis; |
| | | Boschelli, J. Med. |
| | | Chem. 41: 4365 |
| | | (1998) |

Additional PDGF-R inhibitors are described in U.S. Pat. Nos. 5,674,892; 5,728,726; 5,795,910; 5,916,908; 6,331,555; and 6,358,954, incorporated here by reference.

In addition to or in substitution for PDGF-R inhibitors, inhibitors of other tyrosine kinases (receptors and other types as well) may also be used in accordance with this invention. Some of these inhibitors may inhibit multiple kinases including, but not limited to, PDGF-Rs. Appropriate TK inhibitors are also taught in WO 99/03854; WO 01/64200; U.S. Pat. No. 5,521,184; WO 00/42042; WO 00/09098; EP 0 564 409 B1; U.S. Pat. No. 5,521,184; WO 97/32604; U.S. Pat. No. 6,610,688; US Patent Appl. Pub. No. 20030194749; Livitzki, A., et al., “Protein Tyrosine Kinase Inhibitors as Novel Therapeutic Agents,” Pharmacol. Ther. 82:231-29 (1999). Other classes of compounds may also be employed. For example and without limitation, Leflunomide (U.S. Pat. No. 4,284,786) and/or derivative FK778 may be used. (See, e.g., Savikko Transplantation 2003:76:455 and editorial Williams ibid p 471).

F. Smooth Muscle Relaxants

Another class of therapeutics for practice of the invention are molecules that inhibit the function of the SMC/PC cells that have invested themselves in the lymphatic vessels of affected individuals.

One such class of compounds that is contemplated is smooth muscle relaxants that will improve lymphatic flow be de-constricting lymphatic vessels that may be constricted by SMC/PC cells. Such relaxants include, without limitation, antimuscarinic and vasodilators. In a highly preferred embodiment, the relaxant is specific to the vasculature.

Antimuscarinic compounds include the following: adiphenine, ambutonium bromide, aminopentamide, anisotropine methylbromide, atropine, benactyzaine, benzetimide, benzilonium bromide, benztropine mesylate, bevonium methyl sulfate, biperiden, butropium bromide, n-butylscopolammonium bromide, buzepide, chlorbenzoxamine, cimetropium bromide, clidinium bromide, cyclodrine, cyclopentolate, darifenacin, dexetimide, dicyclomine, diethazine, difemerine, dihexyverine, diphemanil methylsulfate, emepronium bromide, ethopropazine, ethybenztropine, eucatropine, fenpiverinium bromide, fentonium bromide, flavoxate, flutropium bromide, glycopyrrolate, hexocyclium methyl sulfate, homatropine, hyoscyamine, ipratropium bromide, isopropamide iodide, mepenzolate bromide, methantheline bromide, methixene, methscopolamine bromide, oxitropium bromide, oxybutynin, oxyphencyclimine, oxyphenonium bromide, penthienate bromide, phenglutarimide, pipenzolate bromide, piperidolate, piperilate, poldine methylsulfate, prifinium bromide, procyclidine, propantheline bromide, propiverine, scopolamine, scopolamine n-oxide, telenzepine, tiemonium iodide, timepidium bromide, tiotropium bromide, tiquizium bromide, tolterodine, tridihexethyl iodide, trihexyphenidyl hydrochloride, tropicamide, trospium chloride, and valethamate bromide.

Vasodilator (cerebral) compounds include the following: bencyclane, ciclonicate, cinnarizine, cyclandelate, diisopropylamine dichloroacetate, eburnamonine, fasudil, fenoxedil, flunarizine, ibudilast, ifenprodil, lomerizine, nafronyl, nicametate, nicergoline, nimodipine, papaverine, pentifylline, vincamine, vinpocetine, and viquidil.

Vasodilator (coronary) compounds include the following: amotriphene, benfurodil hemisuccinate, benziodarone, chloracizine, chromonar, clobenfurol, clonitrate, cloricromen, dilazep, droprenilamine, efloxate, erythrityl tetranitrate, etafenone, fendiline, hexestrol bis(β-diethylaminoethyl ether), hexobendine, itramin tosylate, khellin, lidoflazine, mannitol hexanitrate, nitroglycerin, pentaerythritol tetranitrate, pentrinitrol, perhexiline, pimethylline, prenylamine, propatyl nitrate, trapidil, tricromyl, trimetazidine, trolnitrate phosphate, and visnadine.

Vasodilator (peripheral) compounds include the following: bamethan, bencyclane, beraprost, betahistine, bosentan, bradykinin, brovincamine, bufeniode, buflomedil, butalamine, cetiedil, ciclonicate, cinepazide, cinnarizine, cyclandelate, diisopropylamine dichloroacetate, elecoisin, fenoxedil, flunarizine, hepronicate, ifenprodil, iloprost, inositol niacinate, isoxsuprine, kallidin, kallikrein, moxisylyte, nafronyl, nicametate, nicergoline, nicofuranose, nicotinyl alcohol, nylidrin, pentifylline, piribedil, prostaglandin e1, suloctidil, tolazoline, and xanthinol niacinate.

The above compounds are described in The Merck Index, 13th ed., which is incorporated by reference.

G. Lymphatic Growth Factor Therapy

As discussed in the Examples, the loss of Foxc2 does not result in lymphatic hypoplasia associated with the reduction of VEGFR-3 signaling and this indicates that FOXC2 is not involved in the VEGFR-3 mediated control of lymphatic endothelial proliferation. Rather, FOXC2 controls a functionally separate pathway responsible for the negative regulation of SMC recruitment.

Vascular endothelial growth factors-C and -D (VEGF-C and VEGF-D) are lymphatic growth factors and their use for treating VEGFR-3 heritable lymphedemas is described in International Patent Publication No. WO 00/58511, titled “SCREENING AND THERAPY FOR LYMPHATIC DISORDERS INVOLVING THE FLT4 RECEPTOR TYROSINE KINASE (VEGFR-3).” The entire disclosure of this document is incorporated herein by reference. Use of VEGF-C and VEGF-D protein and gene therapy products as described in this document also is contemplated for treatment of subject with arterialized lymphatics as described herein.

H. Combination Therapy

Use of any of the agents described herein, in combination with any other agent described herein, is specifically contemplated.

In one preferred embodiment, a PDGF/PDGFR inhibitor therapy is administered in conjunction with a lymphangiogenic therapy, such as VEGF-C or VEGF-D therapy. It is contemplated that the latter therapy promotes growth of new lymphatic vessels and the former therapy prevents arterialization of the new vessels.

I. Pharmaceutical Compositions and Routes of Administration

In preferred embodiments, therapeutic agents described herein are formulated in compositions that include at least one pharmaceutically acceptable diluent, adjuvant, or carrier substance, using any available pharmaceutical chemistry techniques. Generally, this entails preparing compositions that are essentially free of pyrogens, as well as other impurities that could be harmful to humans or animals.

One often employs appropriate salts and buffers to render delivery vectors stable and allow for uptake by target cells. Buffers also are employed when recombinant cells are introduced into a patient. Aqueous compositions of the present invention comprise an effective amount of the peptide or an expression vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The active compositions of the present invention include classic pharmaceutical preparations. Administration of these compositions according to the present invention will be via any common route so long as the target tissue is available via that route. The pharmaceutical compositions may be introduced into the subject by any conventional method, e.g., by intravenous, intradermal, intramusclar, intramammary, intraperitoneal, intrathecal, retrobulbar, intrapulmonary (e.g., term release); by oral, sublingual, nasal, anal, vaginal, or transdermal delivery, or by surgical implantation at a particular site. The treatment may consist of a single dose or a plurality of doses over a period of time.

The active compounds may be prepared for administration as solutions of free base or pharmacologically acceptable salts in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle that contains the basic dispersion medium and the required other ingredients from those enumerated above. In the case of sterile powders for the preparation of sterile injectable solutions, the preferred methods of preparation are vacuum-drying and freeze-drying techniques that yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions.

For oral administration the polypeptides of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

“Unit dose” is defined as a discrete amount of a therapeutic composition dispersed in a suitable carrier. For example, where polypeptides are being administered parenterally, the polypeptide compositions are generally injected in doses ranging from 1 μg/kg to 100 mg/kg body weight/day, preferably at doses ranging from 0.1 mg/kg to about 50 mg/kg body weight/day. Parenteral administration may be carried out with an initial bolus followed by continuous infusion to maintain therapeutic circulating levels of drug product. Those of ordinary skill in the art will readily optimize effective dosages and administration regimens as determined by good medical practice and the clinical condition of the individual patient.

The frequency of dosing will depend on the pharmacokinetic parameters of the agents and the routes of administration. The optimal pharmaceutical formulation will be determined by one of skill in the art depending on the route of administration and the desired dosage. See for example Remington's Pharmaceutical Sciences, 18th Ed. (1990, Mack Publ. Co, Easton Pa. 18042) pp 1435-1712, incorporated herein by reference. Such formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein as well as the pharmacokinetic data observed in animals or human clinical trials.

Appropriate dosages may be ascertained through the use of established assays for determining dose-response and toxicity and side-effect data. The final dosage regimen will be determined by the attending physician, considering factors that modify the action of drugs, e.g., the drug's specific activity, severity of the damage and the responsiveness of the patient, the age, condition, body weight, sex and diet of the patient, the severity of any infection, time of administration and other clinical factors. As studies are conducted, further information will emerge regarding appropriate dosage levels and duration of treatment for specific diseases and conditions.

In gene therapy embodiments employing viral delivery, the unit dose may be calculated in terms of the dose of viral particles being administered. Viral doses include a particular number of virus particles or plaque forming units (pfu). For embodiments involving adenovirus, particular unit doses include 10³, 10⁴, 10⁵, 10⁶, 10⁷, 10⁸, 10⁹, 10¹⁰, 10¹¹, 10¹², 10¹³ or 10¹⁴ pfu. Particle doses may be somewhat higher (10 to 100-fold) due to the presence of infection defective particles.

It is known in the art that ex vivo transfection of cells and subsequent transfer of these cells to patients is an effective method to upregulate in vivo levels of the transferred gene and to provide relief from a disease resulting from under-expression of the gene(s) (Gelse et al., Arthritis Rheum. 48:430-41. 2003; Huard et al, Gene Ther. 9:1617-26. 2002; Kim et al., Mol. Ther. 6:591-600 2002). Delivery of a therapeutic composition of the invention to appropriate cells is effected ex vivo, in situ, or in vivo by use of vectors, and more particularly viral vectors (e.g., adenovirus, adeno-associated virus, or a retrovirus), or ex vivo by use of physical DNA transfer methods (e.g., liposomes or chemical treatments). See, for example, Anderson, Nature, supplement to vol. 392, no. 6679, pp. 25-20 (1998). For additional reviews of gene therapy technology see Friedmann, Science, 244:1275-1281 (1989); Verma, Scientific American: 68-84 (1990); and Miller, Nature, 357: 455-460 (1992). Introduction of any one of the polynucleotides of the present invention or a gene encoding the polypeptides of the present invention can also be accomplished with extrachromosomal substrates (transient expression) or artificial chromosomes (stable expression). Transient expression is preferred. Cells may also be cultured ex vivo in the presence of therapeutic compositions of the present invention in order to proliferate or to produce a desired effect on or activity in such cells. Treated cells can then be introduced in vivo for therapeutic purposes.

In a highly preferred embodiment, the ex vivo gene therapy is administered locally, e.g., to the site of edema. For example, the administering comprises a catheter-mediated transfer of the therapeutic composition into a lymphatic vessel of the mammalian subject. Exemplary materials and methods for local delivery are reviewed in Lincoff et al., Circulation, 90: 2070-2084 (1994); and Wilensky et al., Trends Cardiovasc. Med., 3:163-170 (1993), both incorporated herein by reference. For example, the composition is administered using infusion-perfusion balloon catheters (preferably mircroporous balloon catheters) such as those that have been described in the literature for intracoronary drug infusions. See, e.g., U.S. Pat. No. 5,713,860 (Intravascular Catheter with Infusion Array); U.S. Pat. No. 5,087,244; U.S. Pat. No. 5,653,689; and Wolinsky et al., J. Am. Coll. Cardiol., 15: 475-481 (1990) (Wolinsky Infusion Catheter); and Lambert et al., Coron. Artery Dis., 4: 469-475 (1993), all of which are incorporated herein by reference in their entirety. Use of such catheters for site-directed somatic cell gene therapy is described, e.g., in Mazur et al., Texas Heart Institute Journal, 21; 104-111 (1994), incorporated herein by reference.

It will be appreciated that the pharmaceutical compositions and treatment methods of the invention may be useful in fields of human medicine and veterinary medicine. Thus the subject to be treated may be a mammal, preferably human or other animal. For veterinary purposes, subjects include for example, farm animals including cows, sheep, pigs, horses and goats, companion animals such as dogs and cats, exotic and/or zoo animals, laboratory animals including mice rats, rabbits, guinea pigs and hamsters; and poultry such as chickens, turkey ducks and geese.

IV. Synthetic Valves and Medical Devices

Aspects of the invention include materials and methods for transforming vascular or lymphatic cells (especially endothelial cells) with a FoxC2 and/or a Prox1 polynucleotide. The materials include vectors for transformation, and the transformed cells themselves. Cells may be transformed in vitro to improve in vitro valve formation or function. Cells also may be transformed ex vivo and (1) administered into a mammalian subject for the purpose of improving in vitro valve formation or function; (2) administered into a mammalian subject for the purpose of coating a transplanted or an implanted synthetic valve or vessel or stent in the mammalian subject; or (3) used ex vivo to coat a synthetic valve, vessel, stent, or other implantable device prior to its implantation. Synthetic valves, vessels, stents, and other devices coated in this manner are themselves an aspect of the invention. In yet another variation, the synthetic valve is coated with the gene therapy vector to facilitate localized tranfection of endothelial cells upon implantation. These aspects of the invention can be used to improve all synthetic valve technologies, including those in existence and later developed. A number of existing valve technologies that can be improved according to this invention are summarized below with reference to literature, all of which is incorporated by reference in its entirety. Covering venous transplants with endothelial cells is useful to reduce formation of thrombi as well as immunological rejection. Transformation of the cells with FoxC2 and/or Prox1 is expected to reduce the side effect of increased neoinitimal thickening, due to the growth and/or recruitment of fibroblasts or smooth muscle cells, thereby reducing these undesired effects and leading to better acceptance and long term patency of artificial valves.

The use of synthetic venous valves and grafts has been described in the art (U.S. Pat. Nos. 5,397,351 and 6,508,833; Pavcnik et al., J. Vasc. Surgery, 40:121-1227 (2004); Arts et al., Eur. J. Endovasc. Surg., 23:29-38 (2002)).

Replacement of heart valves with prosthetic valves within the human body was perhaps first documented by Hufnagel in 1954 (Hufnagel C. A. Harvey W. P. Rabil P. J. McDermott R. F.: Surgical Correction of Aortic Insufficiency, Surgery, 35:673, 1954). Since then numerous prosthetic valves have been developed, including both mechanical and biological heart valves. Mechanical heart valves are generally of two designs, either a ball-in-cage valve or a disc valve. One example of a mechanical heart valve is that of Boretos, U.S. Pat. No. 3,911,502, which describes a composite heart valve poppet for use with ball-in-cage artificial heart valves. The poppet includes a core of rigid material covered by an elastomer jacket. Leibinsohn, U.S. Pat. No. 3,626,518, discloses an artificial cardiac valve having characteristics of both a ball valve and a disc valve for improved streamlined blood flow and reduced turbulence. Hamaker, U.S. Pat. No. 3,574,865, discloses a prosthetic sutureless heart valve, the heart valve fastening to the heart via a two-piece snap ring. Somyk, U.S. Pat. No. 3,857,815, discloses a suture ring for a heart valve, wherein a cylindrical collar extends radially from the valve to facilitate attachment.

U.S. Pat. No. 5,397,351 describes a prosthetic valve and a method for percutaneous insertion and placement within a fluid passageway of a living body. The valve includes a valve seat, a restraining element, and a poppet. The valve assembly includes a first insertion form for percutaneous insertion into the passageway. The valve assembly includes a second larger operational form for operation within the passageway, wherein the valve seat has means for sealing against the passageway and an opening for sealing engagement with the poppet and the restraining element has means for restraining passage of the poppet therethrough while permitting fluid flow therethrough. The poppet is movably restrained within the passageway between the valve seat and the restraining element, wherein the poppet seals against the opening in the valve seat to prevent fluid flow therethrough and the poppet unseats from the valve seat to permit fluid flow therethrough.

A method for inserting a deformable prosthetic valve within a fluid passageway of a living body is also described in U.S. Pat. No. 5,397,351, incorporated herein by reference. The method includes the steps of placing a deformable aortic prosthetic valve assembly within a distal end of a first sheath, wherein the valve includes a first insertion form and a second larger operational form and the first sheath encompasses the valve when the valve is in the first smaller insertion form, locating the distal end of the first sheath in the passageway, and placing the valve in the passageway by removing the first sheath from the passageway while holding the valve in place.

The “administration” of the composition of the invention may be performed using any medically-accepted means for introducing a therapeutic directly or indirectly into the vasculature of a mammalian subject, including but not limited to injections; oral ingestion; intranasal or topical administration; and the like. In a preferred embodiment, administration of the composition comprising a polynucleotide that encodes FoxC2 or cells transformed with FoxC2 is performed intravascularly, such as by intravenous, intra-arterial, or intracoronary arterial injection. Intradermal and subdermal injection also is contemplanted.

In a highly preferred embodiment, the composition is administered locally, e.g., to the site of edema. For example, the administering comprises a catheter-mediated transfer of the therapeutic composition into a large lymphatic vessel or vein of the mammalian subject. Exemplary materials and methods for local delivery are reviewed in Lincoff et al., Circulation, 90: 2070-2084 (1994); and Wilensky et al., Trends Cardiovasc. Med., 3:163-170 (1993), both incorporated herein by reference. For example, the composition is administered using infusion-perfusion balloon catheters (preferably mircroporous balloon catheters) such as those that have been described in the literature for intracoronary drug infusions. See, e.g., U.S. Pat. No. 5,713,860 (Intravascular Catheter with Infusion Array); U.S. Pat. No. 5,087,244; U.S. Pat. No. 5,653,689; and Wolinsky et al., J. Am. Coll. Cardiol., 15: 475-481 (1990) (Wolinsky Infusion Catheter); and Lambert et al., Coron. Artery Dis., 4: 469-475 (1993), all of which are incorporated herein by reference in their entirety. Use of such catheters for site-directed somatic cell gene therapy is described, e.g., in Mazur et al., Texas Heart Institute Journal, 21; 104-111 (1994), incorporated herein by reference.

In another variation, the composition containing the FoxC2 gene therapy construct is administered extravascularly, e.g., using a device to surround or encapsulate a portion of vessel. See, e.g., International Patent Publications WO 98/20027 and WO 99/55315, incorporated herein by reference, describing a collar that is placed around the outside of an artery (e.g., during a bypass procedure) to deliver a transgene to the arterial wall via a plasmid or liposome vector.

In still another variation, endothelial cells or endothelial progenitor cells are transfected ex vivo with the FoxC2 transgene, and the transfected cells are administered to the mammalian subject. Exemplary procedures for seeding a vascular graft with genetically modified endothelial cells are described in U.S. Pat. No. 5,785,965, incorporated herein by reference.

In another preferred embodiment, the administering comprises implanting an intravascular stent in the mammalian subject, where the stent is coated or impregnated with the FoxC2 genetic construct/vector or the transfected FoxC2 endothelial cells. Exemplary materials for constructing valves, stents or grafts coated or seeded with transfected endothelial cells are described in Pavcnik et al., Eur. J. Endovasc. Surg., 40:1223-1227 (2004); and Arts et al., Eur. J. Endovasc. Surg., 23:29-38 (2002). For example, in one variation, the synthetic valve comprises small intestimal submucosa sutured to a square stainless steel stent. The square stent has a short barb at each end to provide anchors for the valve during placement, and the submucosa membrane is slit at the diagonal axis of the stent to create the valve opening.

Surfaces of the synthetic valve are coated with the transfected endothelial cell, e.g., by placing the synthetic valve in an endothelial cell culture medium for 1-3 days prior to implantation to allow for complete coverage of valve surface with the transfected endothelial cells.

In another preferred embodiment, the administering comprises implanting an intravascular stent in the mammalian subject, where the stent is coated or impregnated is described in literature cited above and reviewed in Lincoff et al., Circulation, 90: 2070-2084 (1994). A metal or polymeric wire for forming a stent is coated with a composition such as a porous biocompatible polymer or gel that is impregnated with (or can be dipped in or otherwise easily coated immediately prior to use with) the transfected Foxc2 endothelial cells. The wire is coiled, woven, or otherwise formed into a stent suitable for implantation into the lumen of a vessel using conventional materials and techniques, such as intravascular angioplasty catheterization. Exemplary stents that may be improved in this manner are described and depicted in U.S. Pat. Nos. 5,800,507 and 5,697,967 (Medtronic, Inc., describing an intraluminal stent comprising fibrin and an elutable drug capable of providing a treatment of restenosis); U.S. Pat. No. 5,776,184 (Medtronic, Inc., describing a stent with a porous coating comprising a polymer and a therapeutic substance in a solid or solid/solution with the polymer); U.S. Pat. No. 5,799,384 (Medtronic, Inc., describing a flexible, cylindrical, metal stent having a biocompatible polymeric surface to contact a body lumen); U.S. Pat. Nos. 5,824,048 and 5,679,400; and U.S. Pat. No. 5,779,729; all of which are specifically incorporated herein by reference in the entirety.

V. Screening Assays

The present invention also contemplates screening of compounds for their ability to promote the growth of a smooth muscle cell-free lymphatic endothelial cell network. The present invention shows that FOXC2 is involved in such a phenotype and shows that when FOXC2 is inactivated PDGF-B is released from lymphatic endothelial cells and causes recruitment of SMCs/PCs to the endothelial cell network. Therefore compositions that inhibit this phenomenon are contemplated to be useful in therapeutic embodiments for the treatment of e.g., lymphedema. Particularly preferred compounds will be those useful in decreasing the interaction and/or activity of PDGF-B and/or its receptor. The present section describes screening assays for identifying such compounds.

In the screening assays of the present invention, the candidate substance may first be screened for basic biochemical activity, and then tested for its ability to increase pulmonary surfactant catabolism, at the cellular, tissue or whole animal level. For the in vitro biochemical assays, one skilled in the art may use any assay designed to monitor the physical or biochemical effects of PDGF and/or FOXC2. For example, those of skill in the art will be aware of the types of assays that can be used to determine the activity of a given receptor/ligand (e.g., PDGF-B/PDGFR-β) or the activity of FOXC2 in inhibiting this receptor/ligand interaction. Binding assays are described in PCT/IB02/00099 for determining the binding of peptides to VEGFR-3, setting up an assay for determining the binding of peptides to PDGFR-β would be analogous and as such, the entire description in Section C of the detailed description of the preferred embodiments of PCT/IB02/00099 is incorporated herein by reference. In addition, the present application may employ biological activity assays to monitor the activity of FOXC2, PDGF-B or a receptor of PDGF-B. Such biological assays include endothelial cell migration assays, assays for effects on vascular permeability, endothelial cell proliferation assays, choroallantoic membrane (CAM) assays and the like. Exemplary such assays are described in PCT/IB02/00099, which is incorporated herein by reference as providing a teaching of those assays.

For the in vivo testing, there are various animal models of edema in general and models of LD are specifically described in the Examples herein (e.g., mice with mutations in FoxC2 and/or mutations in VEGFR-3). Such animal models may be used to test the in vivo effects of the compounds selected using in vitro assays.

A. Modulators and Assay Formats

The present invention provides methods of screening for agents that promote the growth of SMC free lymphatic endothelial cells. It is contemplated that such screening techniques will prove useful in the identification of compounds that will augment, stimulate or otherwise increase the effects of FOXC2 in inhibiting PDGF-B induced recruitment of SMCs to an endothelial cell network and as such will be useful in the treatment of lymphedema in general and LD specifically. In these embodiment, the present invention is directed to a method for determining the ability of a candidate substance to increase the growth of an endothelial cell network without producing an increased recruitment of SMCs, generally including the steps of:
a. obtaining a sample comprising vascular smooth muscle cells and lymphatic endothelial cells that comprise a mutation in FoxC2 and determining the association of said vascular smooth muscle cells with said lymphatic endothelial cells to determine a baseline association; and
b. contacting the sample of step (a) with a candidate substance and determining the association of said vascular smooth muscle cells with said lymphatic endothelial cells in the presence of said candidate substance; wherein an increase in the degree of said association of said vascular smooth muscle cells with said lymphatic endothelial cells in the presence of said candidate substance as compared to the degree of association of said vascular smooth muscle cells with said lymphatic endothelial cells in the absence of said candidate substance is indicative of said candidate substance being an agent that promotes the growth of a smooth muscle cell free lymphatic endothelial cell network.

The FOXC2 may be an isolated fraction or may be within a cell.

To identify a candidate substance as being capable of promoting the growth of a smooth muscle cell-free lymphatic endothelial cell network in the assay above, one would measure or determine the presence of growth of the SMCs in co-culture with the endothelial cells in the absence of the added candidate substance. One would then add the candidate substance to the co-culture cell and determine the response of the co-culture in the presence of the candidate substance. A candidate substance which decreases the association of the SMCs with the endothelial cells in the co-culture is indicative of a candidate substance having the desired activity. In the in vivo screening assays of the present invention, the compound is administered to a model animal, over period of time and in various dosages, and an alleviation of the symptoms associated with edema are monitored. Any improvement in one or more of these symptoms will be indicative of the candidate substance being a useful agent.

In other embodiments, the assay is set up such that the method comprises:
a. obtaining a sample comprising vascular smooth muscle cells and lymphatic endothelial cells that comprise a mutation in FoxC2 and determining the association of said vascular smooth muscle cells with said lymphatic endothelial cells to determine a baseline association;
b. contacting the sample of step (a) with a composition comprising FoxC2 and determining the association of said vascular smooth muscle cells with said lymphatic endothelial cells in the presence of said FoxC2 to determine the degree of decrease in association of said vascular smooth muscle cells with said lymphatic endothelial cells as compared to said baseline;
c. contacting the sample of step (b) with a candidate substance and determining the association of said vascular smooth muscle cells with said lymphatic endothelial cells in the presence of said candidate substance; wherein a decrease in the degree of association of vascular smooth muscle cells with said lymphatic endothelial cells in step (c) as compared to step (b) is indicative of said candidate substance being an agent that stimulates the activity of FoxC2 as a promoter of muscle cell-free lymphatic endothelial cell network.

As used herein the term “candidate substance” refers to any molecule that may potentially act as an inhibitor of the PDGF/PDGFR-3 interaction. As discussed above, such an agent may be a protein or fragment thereof, a small molecule inhibitor, or even a nucleic acid molecule. It may prove to be the case that the most useful pharmacological compounds will be compounds that are structurally related to other known inhibitors of PDGF-B. For example, tyrosine kinase inhibitors are well known to those of skill in the art and may prove useful agents in the screening teachings taught herein. Rational drug design includes not only comparisons with known such inhibitors, but predictions relating to the structure of target molecules of such inhibitors.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds molded of active, but otherwise undesirable compounds.

Candidate compounds may include fragments or parts of naturally-occurring compounds or may be found as active combinations of known compounds which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or manmade compounds. Thus, it is understood that the candidate substance identified by the present invention may be polypeptide, polynucleotide, small molecule inhibitors or any other compounds that may be designed through rational drug design starting from known tyrosine kinase inhibitors or other inhibitors of PDGF-B/PDGFR-β action.

“Effective amounts” of the candidate agent in certain circumstances are those amounts effective to reproducibly produce an alteration in the inhibition of PDGF-B expression or activity, inhibition of PDGFR-β expression or activity, growth of SMCs, expression or activity of FOXC2 in comparison to the levels of these parameters in the absence of such an agent normal levels. Compounds that achieve significant appropriate changes in activity and/or expression of PDGF-B will be used.

Significant changes in activity and/or expression will be those that are represented by alterations in activity of at least about 30%-40%, and most preferably, by changes of at least about 50%, with higher values of course being possible.

B. In vitro Assays

A quick, inexpensive and easy assay to run is a binding assay. Binding of a molecule to a target may, in and of itself, be stimulatory, due to steric, allosteric or charge-charge interactions. This can be performed in solution or on a solid phase and can be utilized as a first round screen to rapidly eliminate certain compounds before moving into more sophisticated screening assays. In one embodiment of this kind, the screening of compounds that bind to the PDGF-B or fragment thereof is provided.

The target may be either free in solution, fixed to support, expressed in or on the surface of a cell. Either the target or the compound may be labeled, thereby permitting determining of binding. In another embodiment, the assay may measure the inhibition of binding of a PDGF-B to PDGFR-β. Competitive binding assays can be performed in which one of the agents, e.g., the substrate or the candidate substance is labeled. Usually, the target will be the labeled species, decreasing the chance that the labeling will interfere with the binding moiety's function. One may measure the amount of free label versus bound label to determine binding or inhibition of binding.

A technique for high throughput screening of compounds is described in WO 94/03564. Large numbers of small peptide test compounds are synthesized on a solid substrate, such as plastic pins or some other surface. The peptide test compounds are reacted with, for example, PDGFR-β and washed. Bound polypeptide is detected by various methods.

Purified target, such as PDGFR-β can be coated directly onto plates for use in the aforementioned drug screening techniques. However, non-neutralizing antibodies to the polypeptide can be used to immobilize the polypeptide to a solid phase.

C. In vivo Assays

The present invention particularly contemplates the use of various animal models of lymphedema. As discussed above, there are well-characterized transgenic mice models of this disorder and these may be used for screening assays in a whole animal system. This animal model can, therefore, be used not only screen for modulators of FOXC2 activity, but also to track the therapeutic effects of the candidate substance in the treatment of lymphedema.

Treatment of these animals with test compounds will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection.

Determining the effectiveness of a compound in vivo may involve a variety of different criteria. Such criteria include, but are not limited to, survival, reduction of protein excretion, and improvement of general physical state including activity. It also is possible to perform histologic studies on tissues from these mice, or to examine the molecular and morphological state of the cells, which includes cell size, other morphological indicators or alteration in the expression of genes involved in surfactant disorders.

VI. Determination of Lyphedema Distichiasis Phenotypic Traits and Identification of Pathways for Therapeutic Targeting

The experiments described in this section were performed to better characterize the pathogenesis of lymphedema distichiasis. The experiments identified a number of heretofore unappreciated physiological phenomena and biochemical processes that represent new targets for therapeutic intervention.

A. Materials and Methods

Following is a brief description of reagents and techniques used.

Antibodies. The monoclonal antibodies used herein include rat anti-mouse FOXC2 (Furumoto et al., Dev Biol 210, 15-29, 1999), mouse anti-human FOXC2 (Yang et al., FEBS Lett 470, 29-34, 2000), rat anti-mouse PECAM-1 (Pharmingen), mouse Cy3-conjugated anti-human SMA (Sigma), rabbit anti-human vWF (DAKO), mouse anti-human c-myc (Babco), biotinylated rabbit anti-mouse VEGFR-3 (R&D), rabbit anti-human podoplanin (Breiteneder-Geleff et al., Am. J. Pathol., 154, 385-394, 1999) and rabbit anti-mouse NG2 (Ozerdem et al., Dev Dyn 222, 218-27, 2001). Rabbit anti-mouse LYVE-1 for whole mount staining was produced using baculoviral LYVE-1 extracellular domain-human Fc fusion protein, and rabbit anti-mouse LYVE-1 for immunohistochemical staining was that described in Laakkonen et al., (Nat Med 8, 751-5, 2002). The fluorochrome- and HRP-conjugated secondary antibodies were obtained from Jackson Immunoresearch Laboratories, Molecular Probes and Vector Laboratories.

Mice breeding and genotyping Foxc⁺/⁻, Vegfr-2⁺/^{lacZ}, Vegfr-3⁺/^{lacZ} mice were bred on C57B16J background, and mice for breeding were obtained from Harlan Laboratory. To obtain E17.5 Foxc2⁻/⁻ embryos Foxc2⁺/⁻ mice were crossed with ICR mice, and F1 mice were used for breeding. Wt and Foxc⁺/⁻ littermates were used as the controls. Mice were genotyped using Southern blot or PCR procedures as described previously Iida, K. et al. Essential roles of the winged helix transcription factor MFH-1 in aortic arch patterning and skeletogenesis. Development 124, 4627-38. (1997; Dumont, D. J. et al. Cardiovascular failure in mouse embryos deficient in VEGF receptor-3. Science 282, 946-949 (1998) or by β-galactosidase staining, when analyzing Vegfr-3⁺/^{lacZ} and Vegfr-2⁺/^{lacZ} E17.5 embryos.

Analyses of the lymphatic and blood vessels. Whole mount immunohistochemistry staining of LYVE-1 and SMA were performed on 4% PFA fixed tissues. For β-galactosidase staining, tissues were fixed in 0.05% glutaraldehyde and stained using X-gal substrate (Sigma). For visualization of functional lymphatic vessels, mice were anesthetized and Evans blue dye (Sigma, 5 mg/ml) was injected into the footpad of the hindlimb or FITC-dextran (Sigma, 8 mg/ml) was injected intradermally into the ear. Lymphatic vessels were analyzed by light of fluorescence microscopy.

Cloning procedures. To produce FOXC2 expression vector, full coding region for FOXC2 from IMAGE clone 2758434 was subcloned into pAMC vector to produce WT FOXC2/pAMC construct encoding FOXC2 protein tagged with myc at the N-terminus. To introduce the mutations, regions containing the mutations were amplified by PCR using DNA isolated from the LD patients and cloned into pCR-TOPO II vector (Invitrogen). Clones containing the expected mutation were identified by sequencing, and further subcloned into the corresponding region of the WT FOXC2/pAMC. The notation of the mutations was the same as in Finegold et al., (Hum. Mol. Gen. 10, 1185-1189, 2001) and the following mutants were used in the study: mutant A—14 bp duplication-insertion at the nucleotide 232 of FOXC2 cDNA (GeneBank accession number NM_{—}005251), termination at amino acid 100; mutant B—C→T at the nucleotide 252, Q→Term at a amino acid 84; mutant C—deletion A at 505, termination at amino acid 202; mutant F—insertion C 609, termination at amino acid 463; mutant K—16 bp deletion at 1238, termination at amino acid 427. Mutants A, B and C have truncated DNA binding domains, whereas mutants F and K contain intact DNA binding domains. The inserts were verified by sequencing, and the correct protein products were detected upon transient transfection of 293T cells, as determined by western blotting using the antibodies against c-myc or FOXC2 (for the WT protein).

To produce FOXC2 responsive reporter, oligonucleotides 5′-GATCCTTAAGTAAGTAAACAAACAAGATCCTTAAGTAAGTAAACAAACAA GATC-3′ (SEQ ID NO: 25) and 5′-GATCTTGTTTGTTTACTTACTTAAGGATCTTGTTTGTTTACTTACTTAAGGA T-3′ (SEQ ID NO: 26) containing two FOXC2 binding sites were annealed and cloned into Hind III site of pTAL-luc, which is designed for the analysis of the enhancer sequences (Clontech). The cloning was repeated two more times to produce a plasmid 6×FOXC2/pTAL-luc containing six FOXC2 binding sites. Other commonly used reporters such as pGL3 vectors were not as suitable for the analysis of FOXC2 activity, as they are efficiently transactivated even in the absence of FOXC2 binding sites.

For GAL4 heterologous transcriptional activation assay, fragments of FOXC2 cDNA corresponding to the amino acids 237-501, 338-501, and 414-501 were subcloned into pM1 vector (Clontech), to produce C-term 264/pM1, C-term 163/pM1 and C-term 87/pM1 plasmids encoding fusion proteins with GAL4 DNA binding domain (DBD). Junctions were verified by sequencing to confirm that FOXC2 fragments are cloned in frame with GAL4 DBD.

RNA isolation and Northern blotting. Total RNA was isolated using RNeasy columns (Qiagen). For Northern analysis, 5 μg of total RNA was separated in 1% agarose gels and the blots were hybridized in Ultrahyb solution (Ambion) with ³²P-labelled BsrGI/BamHI 702 bp fragment of human FOXC2 which does not cross-hybridizes with human FOXC1 (nucleotides 2063-1361 of IMAGE clone 2758434). Probes for PROX1 and STAT6 were described previously (Petrova et al., EMBO J. 21, 4593-9, 2002).

Cell culture and transfection. Human primary coronary artery, saphenous vein and microvascular endothelial cells were from Promocell. Lymphatic endothelial cells were isolated and cultured as described previously Makinen, T. et al. Isolated lymphatic endothelial cells transduce growth, survival and migratory signals via the VEGF-C/D receptor VEGFR-3. Embo J 20, 4762-73. (2001), and were more than 98% pure as determined by staining for lymphatic endothelial markers podoplanin. HepG2 and 293T cells were cultured in DMEM, containing 10% fetal bovine serum, 1 mM glutamine and antibiotics. For the luciferase assays transfections were carried out using calcium phosphate method according to the manufacturer's instructions (Gibco). HepG2 cells were co-transfected with the firefly luciferase reporter constructs 6×FOXC2/pTAL or pTAL and the indicated amounts of WT FOXC2, mutant expression vectors or empty expression vector. To study trans-activation properties of FOXC2 C-terminus, 293T cells were transfected with pG5-luc reporter construct containing five GAL4 binding sites (Promega) and GAL4 DNA binding domain fusion constructs C-term 264/pM1, C-term 163/pM1 and C-term 87/pM1 or empty pM1. Renilla liciferase reporter pRL-TK was used to normalize the transfection efficiency (Promega). 36 h after the transfection cell lysates were analyzed for the luciferase activity using Dual-Luciferase™ kit according to the manufacturer's instructions (Promega, Madison, Wis.).

Immunohistochemical staining and in situ hybridization. E14.5 mouse embryo were fixed with 4% paraformaldehyde, dehydrated and embedded in paraffin. 5-μm sections were stained with antibodies against PECAM-1 or lymphatic vessel endothelial hyaluronan receptor-1 LYVE-1, using a Tyramide Signal Amplification kit (TSA, NEN). For double immunofluorescent staining and RNA in situ hybridization, tissues were embedded in OCT (Sakura TissueTek) and frozen at −70° C. For immunofluorescent staining 6 μm sections were fixed in −20° C. methanol for 10 min. Specificity of FOXC2 antibody was confirmed by the absence of staining in Foxc2⁻/⁻ tissues.

For double immunofluorescent staining, 5-μm sections of skin from the LD patients and controls were incubated with rabbit antibodies against podoplanin, followed by incubation with Alexa488 conjugated anti-rabbit antibodies and Cy3-conjugated antibodies against SMA. The SMA postive lymphatic vessels were counted by two blinded observers, using 3-5 sections from each specimen.

For in situ hybridization 15 μm frozen sections were fixed in 4% paraformaldehyde and processed for in situ hybridization as described elsewhere. In In situ hybridization: a practical approach (ed. Wilkinson, D. G.) (Oxford University Press, Oxford New York Tokyo, 1998). The following mRNA probes were used: PDGFRβ PDGF-B (Lindahl et al., Science 277, 242-5, 1997), RGS5 (Bondjers et al. Am J Pathol 162, 721-9, 2003) FOXC2 (nucleotides 1254-2007 of NM_{—}013519.1), and PROX1 (nucleotides 276-1208 of NM_{—}002763.3). Following in situ hybridization, lymphatic vessels were visualized using the antibody against VEGFR-3 and the TSA procedure. Whole mount in situ hybridization of embryos and mesenteric vessels was performed as described. In situ hybridization: a practical approach (ed. Wilkinson, D. G.) (Oxford University Press, Oxford New York Tokyo, 1998). PDGF-B positive lymphatic vessels were counted by two blinded observers using 3-5 sections from 3-4 embryos of each genotype. The statistical significance was analyzed using Student's t-test with P<0.05.

B. Observations and Analysis

1. Abnormal Lymphatic Vasculature and Enhanced Pericyte/Smooth Muscle Cell Recruitment in Foxc2⁻/⁻ Mice

The impairment of lymphatic vasculature or lymphatic flow in the Foxc2 heterozygous mice in the C57B16J background were not demonstrated in whole mount immunohistochemistry, fluorescent or Evans blue microlymphangiography (supplementary figure S1 and not shown). The density of branchpoints in the lymphatic vasculature of the ear was also similar in the wt and Foxc2^{+/-} mice. However, in agreement with Kriederman et al., distichiasis was observed in 100% of Foxc2^{+/-} mice (n=15). The lymphatic vasculature was investigated in Foxc2^{-/-} embryos at E14.5, at the stage when lymphatic vessels have already developed and are growing. These Foxc2^{-/-} mice were edematous, whereas the heterozygous embryos were similar to the wild type (wt) embryos. E14.5 Foxc2−/− but not the wt littermate control embryos display edema however, the overall development of the lymphatic vessels was not affected. For these determinations, the histochemical sections from the E14.5 Foxc2−/− and littermate control embryos were stained with antibodies against lymphatic endothelial marker LYVE-1.

Immunohistochemical staining for the lymphatic endothelial hyaluronan receptor LYVE-1 in the Foxc2^{-/-} mice revealed virtually normal density and patterning of the major lymphatic vessels, including the thoracic ducts, lymphatic plexuses and jugular lymphatic sacs, although the latter were distended in comparison to the wild type mice. These data suggest that Foxc2 is dispensable for early lymphatic development, and that the transient edema may be due to the abnormal cardiovascular development reported earlier (Iida et al., Development, 124, 4627-38, 1997; Winnier et al., Dev. Biol. 213, 418-431 (1999).

The lymphatic vasculature was further studied at E17.5, when extensive sprouting of lymphatic capillaries occurs in the skin. Staining for LYVE-1 demonstrated enlarged lymphatic vessels in the skin of Foxc2^{-/-} embryos, whereas no difference was seen between the Fox2^{+/-} and the wt embryos. The lymphatic endothelium and vascular smooth muscle cells (SMCs) were further investigated by whole mount staining for LYVE-1 and smooth muscle actin (SMA) in the embryos at E17.5. While no difference was seen between the Foxc2^{+/-} and littermate wt control mice, the lymphatic capillaries in the skin of the Foxc2^{-/-} mice were distended and highly tortuous. The density and morphology of blood vessels, identified by staining for the pan-endothelial marker PECAM-1, was similar in all samples (supplementary data S2). Strikingly, all dermal lymphatic capillaries in the Foxc2^{-/-} mice had associated periendothelial cells positive for SMA, whereas the normal lymphatic vessels lacked SMCs/pericytes (PC). Staining for the PC specific proteoglycan NG2 (Ozerdem et al., Dev Dyn 222, 218-27, 2001) demonstrated that the lymphatic vessels in the Foxc2 deficient mice were surrounded by numerous NG2 positive periendothelial cells, some of which were also SMA positive.

Signaling through receptor tyrosine kinase PDGFR-β is essential for the normal recruitment of PCs by developing blood capillaries (Lindahl et al., Science 277, 242-5 1997; Hellstrom et al., Development 126, 3047-55, 1999). PDGFR-β expressing cells were intimately associated with VEGFR-3 positive lymphatic vessels in Foxc2^{-/-} mice, whereas expression of RGS5, a novel marker for some types of PCs (Bondjers et al., Am J Pathol 162, 721-9, 2003), was not observed in the periendothelial cells of the lymphatic vessels. Moreover, about 50% of cutaneous lymphatic vessels in Foxc2^{-/-} mice expressed PDGFR-β ligand PDGF-B, whereas only 5-10% of lymphatic vessels were positive for PDGF-B in the skin of wt and heterozygous embryos.

2. FOXC2 and VEGFR-3 Cooperate in Lymphatic Vascular Patterning and PC Recruitment

In order to visualize the lymphatic vessels in the Foxc2 heterozygous mice more effectively, Foxc2 mice were crossed with Vegfr3⁺/^{LacZ} mice, which have a 3-galactosidase gene at the Vegfr-3 locus. During early embryonic development VEGFR-3 is expressed in blood vessels, but its expression becomes restricted to the developing lymphatic endothelium after E12.5, thus providing a convenient marker for this cell type (Kaipainen et al., Proc. Natl. Acad. Sci. USA 92, 3566-3570, 1995; Dumont et al., Science 282, 946-949, 1998). There was no difference in the patterning of the developing lymphatic vessels between Foxc2^{+/-}/Vegfr3^{+/LacZ} and Foxc2^{+/+}/Vegfr-3^{+/LacZ} embryos at E12.5, suggesting that the formation of primitive lymph sacs and early lymphatic sprouting proceeds normally at this stage. Unexpectedly however, the Foxc2^{+/-}/Vegfr3^{+/LacZ} compound heterozygous embryos at E14.5 displayed mild edema. Staining for β-galactosidase revealed enlarged lymphatic capillaries with fewer sprouts in the nuchal area than in the Foxc2^{+/+}/Vegfr3^{+/LacZ} embryos. Furthermore, at E17.5 100% of the Foxc2^{+/-}/Vegfr3^{+/LacZ} compound embryos showed defects of cutaneous lymphatic capillaries that were similar to those in the Foxc2^{-/-} embryos, i.e. highly tortuous abnormally patterned lymphatic vessels. Increased PC/SMC investment was observed in 70% of the cases. No defects of lymphatic vascular development were observed in Vegfr3^{+/LacZ} embryos. As the lymphangiogenic growth factor VEGF-C binds to and activates both VEGFR-2 and VEGFR-3, and both receptors are expressed in lymphatic vessels (Saaristo et al., J Exp Med 196, 719-30, 2002), Foxc2^{+/-}/Vegfr-2^{+/LacZ} embryos was also analyzed. However, no difference in the lymphatic vessel patterning or SMCs/PC recruitment was observed in these mice, demonstrating that FOXC2 interacts specifically with VEGFR-3 signaling pathway.

VEGFR-3 mRNA and protein were not down-regulated in Foxc2^{-/-} mice, suggesting that FOXC2 may act downstream of VEGFR-3. Vegfr-3^{-/-} embryos die at E10, thus precluding the analysis of FOXC2 expression in developing lymphatic vessels. However, at E9.5 FOXC2 and VEGFR-3 are co-expressed in the heart endothelium. Expression of FOXC2 is significantly lower in the heart endothelium of Vegfr-3^{-/-} embryos in comparison to the controls, consistent with the possibility that, at least in this cell type, VEGFR3 controls the expression of FOXC2.

3. Agenesis of Lymphatic Valves in Foxc2^{-/-} Mice

Injection of FITC-dextran into the hindlimbs of E17.5 Foxc2^{-/-} embryos demonstrated abnormal outflow from the collecting lymphatic vessels to the procollector vessel branches, whereas only the collecting lymphatic vessel trunks were visualized in control embryos. The retrograde filling of lymphatic vessels in Foxc2^{-/-} mice suggested that they had defective valves. Indeed, the valves were seen as fluorescence-negative cusps in the FITC-dextran filled collecting vessels of wildtype but not Foxc2^{-/-} mice. Similarly, the mesenteric vessels of Foxc2^{-/-} mice lacked valves, whereas those of wild type embryos contained 12+/−4.3 valves.

4. FOXC2 is Expressed in Lymphatic but not Blood Vasculature of the Skin

FOXC2 expression has been previously described in arterial endothelial cells and surrounding smooth muscle cells of developing mouse embryos and in cultured human arterial smooth muscle cells (Kume et al., Genes Dev 15, 2470-82, 2001). It was discovered that FOXC2 is also expressed in primary human lymphatic endothelial cells, as well as in cultured blood vascular endothelial cells such as coronary artery, saphenous vein and microvascular endothelial cells. Double immunofluorescent staining of E17.5 mouse skin with antibodies against FOXC2 and LYVE-1 demonstrated that FOXC2 is expressed in the developing lymphatic vessels. In contrast, virtually no expression was seen in skin blood vessels or surrounding SMCs, as determined by double staining for FOXC2 and von Willebrand factor or SMA. In agreement with previously published data¹⁹-21 (Kume et al., Genes Dev 15, 2470-82, 2001; Kaestner et al., Development 122, 1751-8, 1996; Miura et al., Genomics 41, 489-92, 1997). FOXC2 was strongly expressed in the endothelium and SMCs of the vena cava and the carotid artery. The expression pattern of FOXC2 in the skin vasculature strongly suggests that the abnormal lymphatic vascular patterning and PC/SMCs recruitment in Foxc2^{-/-} mice are the results of lymphatic endothelial cell autonomous defects.

5. Increased SMCs/PCs Coverage of the Lymphatic Vessels in the Skin of LD Patients.

The following experiments demonstrate that abnormal recruitment of SMCs/PCs to the lymphatic vasculature occurs in human disease.

Double staining for lymphatic endothelial marker podoplanin and SMA demonstrated that 80% of lymphatic vessels were covered with SMA positive cells in foot skin biopsies from LD patients, whereas less than 10% of lymphatic vessels contained associated SMA positive cells in the control samples. No SMA expressing cells were found in association with the lymphatic vessels in biopsies taken from the unaffected forearm.

6. Functional Analysis of the LD Mutations.

The present example shows that mutations in FOXC2 were associated with a disease phenotype. In this example, the consequences of the LD mutations on the transcriptional activity of FOXC2 is demonstrated. Consistent with the role of FOXC2 in LD, mutated FOXC2 alleles isolated from lymphedema patients failed to trans-activate a synthetic 6×FOXC2-luc target promoter in reporter assays, while the human wt allele showed the appropriate activity. Interestingly, FOXC2 mutants were unable to repress the activity of the wt protein, confirming that FOXC2 haploinsufficiency underlies the clinical manifestations of LD. The isolated C-terminal fragments of FOXC2 fused to the GAL4 DNA-binding domains showed potent transactivation of the heterologous GAL4-dependent promoter, which further confirms the location of the transactivation domain in FOXC2 protein and is consistent with deletion of the C-terminus from most of the inactive FOXC2 alleles in humans (Fang et al., Am. J. Hum. Gen. 67, 1382-1388, 2000; Finegold et al., Hum. Mol. Gen. 10, 1185-1189, 2001; Brice et al., J Med Genet 39, 478-83, 2002).

7. Additional Analysis

The above studies demonstrate the pathogenetic mechanism for human LD and provide the first report of an enhanced recruitment of PC/SMCs to cutaneous lymphatic capillaries. More particularly, it is demonstrated that the SMC chemoattractant PDGF-B is under the control of a FOXC2 signaling pathway in the lymphatic, but not in blood vessels. Additionally, it is shown that FOXC2 and VEGFR-3 interact with the pathway blocking smooth muscle cell recruitment. These results are particularly unexpected because while previous studies have demonstrated that FOXC2 deficient phenotype include abnormalities of the aortic arch, heart, skeletal and kidney development, there has been no indication that deficiencies in this gene are associated with vascular smooth muscle cell phenotype has been reported.

The above studies also demonstrate that Foxc2 is essential for the development of lymphatic valves and that the persistent expression of Foxc2 in adult valves suggests a role in the maintenance of their function. Notably, lymphatic vessel segments containing valves are devoid of a SMC layer, possibly to permit unidirectional lymph propulsion during regular contraction of the lymphangions. Perhads Foxc2 needs to be continuously expressed in the valves to prevent the investment of these regions by mural cells.

Like Foxc2-deficient mice, individuals with LD showed markedly increased recruitment of PC/SMCs to lymphatic capillaries and lymph back flow, indicating abnormalities of the collecting lymphatic vessels. Defective lymphatic valves and abnormal recruitment of the PC/SMCs may indeed be the key mechanisms underlying the clinical manifestations of LD. This could lead to impaired permeability of the lymphatic vessel wall to the protein-rich interstitial fluid, impaired flow of lymph within the lymphatic capillaries because of the uneven luminal size, uncoordinated contractility of the SMCs, and finally reflux of lymph in the collecting vessels resulting from valve incompetence. Althogether, these changes could be responsible for the characteristic impairment of lymphatic drainage seen on clinical lymphangiography of individuals with LD.

Additionally, recruitment of SMCs to blood vessels proceeds normally in the FOXC2 homozygous embryos and even in the compound homozygous Foxc2; Foxc1 embryos, which generally display more severe vascular defects than the single homozygous animals. The above studies show that skin lymphatic vessels, unlike blood vessels, do not produce PDGF-B, a growth factor essential for the recruitment of PCs to blood vasculature, unless Foxc2 is inactivated. These results teach that FOXC2 is required for the establishment of a SMC-free lymphatic capillary network through the suppression of PDGF-B expression. Interestingly, increased expressionf of PDGFR-alpha and -beta and VEGF have been reported in the skin of patients with CVI (see Peschen et al., Arch. Dermatol. Res., 290:291-297, 1998; Quatresooz et al., Intl. J. Mol. Med., 11:411-518, 2003).).

In the skin the expression of FOXC2 is restricted to lymphatic vessels, without being bound to any particular theory or mechanism of action, it is thought that the lymphatic vascular defects in Foxc2^{-/-} mice are very likely to be cell autonomous, and not due to a defective PC/SMC phenotype. This is further supported by the fact that there is an abnormal patterning of lymphatic vessels accompanied by the increased recruitment of SMCs in compound Foxc2^{+/-}/Vegfr-3^{+/LacZ} mice, suggesting a role for FOXC2 as acting downstream of VEGFR-3 as a negative regulator of the PC/SMC coverage in the lymphatic capillaries. Thus, it is asserted that decreased VEGFR-3 signaling in lymphatic endothelial cells results in the inhibition of FOXC2 expression or transcriptional activity, which, in turn, is a pre-requisite for the successful interaction of lymphatic endothelial cells and SMCs. Consistent with this view, VEGFR-3 levels are down-regulated in the collecting lymphatic vessels, which contain a SMC layer. The fact that loss of Foxc2 does not result in lymphatic hypoplasia associated with the reduction of VEGFR-3 signaling suggests that FOXC2 is not involved in the VEGFR-3 mediated control of lymphatic endothelial proliferation. Rather, FOXC2 controls a functionally separate pathway responsible for the negative regulation of SMC recruitment. This finding will be useful in providing combination therapy treatments for lymphedema where a first composition is based on interceding in the FOXC2 pathway and the second therapy is based on interceding in the VEGFR3 pathway.

Lymphatic vessels in Foxc2^{-/-} mice are highly tortuous and display aneurism-like structures, suggesting that the interaction of lymphatic endothelial cells with surrounding extracellular matrix (ECM) is disturbed. In normal skin initial lymphatic capillaries are linked to the surrounding ECM by anchoring filaments, which are composed of the fibrillin and elastin. Here it is possible that the loss of Foxc2 may lead to the absence or abnormal formation of anchoring filaments. Without being bound to any theory or mechanism of action, it may be that FOXC2 could act through the regulation of the expression of fibrillin and elastin or the adhesion molecules shown to be present at the sites of the attachment of anchoring filaments to lymphatic endothelial cells, such as integrins α2β1, α3β3 and αvβ3, vinculin, talin and focal adhesion kinase. Alternatively, the production of ECM components, such as laminins, collagens, fibulins, nidogen and others could be affected in lymphatic vessels of Foxc2^{-/-} mice. Enhanced production of ECM components and formation of basal lamina around lymphatic capillaries would impair the normal uptake of interstitial fluid and compromise the lymphatic vascular function. Interestingly, transforming growth factor beta (TGFβ), one of the known regulators of ECM protein production, induces the expression of FOXC2, suggesting that this transcription factor is mediating some of the TGFβ effects. The expression of adhesion molecules and ECM components in Foxc2^{-/-} mice and the controls using suitable antibodies and the in situ hybridization techniques can be determined using techniques known to those of skill in the art to further corroborate the involvement of FOXC2 in these cellular sequelae. Further the morphological structure of lymphatic vessels and their anchoring filaments may be assessed using electron microscopy and other gross morphology techniques known to those of skill in the art to verify the involvement of FOXC2 in regulating the interaction of lymphatic vessels with ECM components.

### EXAMPLE 1

Mice heterozygous for Foxc2 and the lymphatic endothelial receptor VEGFR-3 display abnormal lymphatic vascular patterning and increased pericyte/smooth muscle cell investment of lymphatic capillaries. Such mice are administered therapeutic agents described herein at varying doses and combinations, and evaluated for lymphatic flow or backflow or edema before and after treatment. Tissue samples are evaluated before and after treatment for general appearance and density of lymphatic vessels and for amount of SMC investment in the lymphatic capillaries and for PDGF and PDGFR expression.

Additional procedures for determining the effects of the therapy include lymphoscintigraphy. In such analyses, a Tc-99m filtered colloid, at a dose of 50 μCi, is injected into the tail vein, ear or other site of the animal at which a swollen phenotype is observable. Imaging of the injection is performed using a large field-of-view Genesys γ camera (ADAC Laboratories, Milpitas, Calif., USA). To qualitatively compare the drainage of the injected radiotracers the radioactivity at the injection sites is counted. The γ counts compared between a group of treated animals and a group of untreated animals.

In addition to monitoring the thickness of the extremities and drainage in response to the therapy, other characteristics may be monitored. For example, as described in Example 1 herein above, the animals may be sacrificed and a histological examination of the blood and lymphatic vessels may be obtained. Such histological examination also may determine skin thickness of animals that have and have not been treated with the therapeutic compounds.

A composition of the present invention is typically administered orally or parenterally in dosage unit formulations containing standard, well known non-toxic physiologically acceptable carriers, adjuvants, and vehicles as desired. The term parenteral as used herein includes subcutaneous injections, intravenous, intramuscular, intra-arterial injection, or infusion techniques. The composition may be delivered to the animal alone or indeed in combination with any of the other therapies discussed herein throughout.

A typical treatment course may comprise about multiple doses delivered daily or several times a day.

The experiments are repeated in other animal models, including animals identified or created with a FoxC2 mutation and animal models with LD phenotypic characteristics. Experiments in rabbits, dogs, pigs, and primates are contemplated, for example.

These animal models are used to identify the initial dosage ranges and protocols for the treatment of higher mammals, such as primates, and particularly humans. In such higher animals, the clinician may chose a regimen to be continued over a period of time, e.g., weeks or months.

Clinical responses in both the test animals and the human subjects being treated may be defined by any acceptable measure that is indicative of lymphedema. For example, a complete response may be defined by the disappearance of all measurable swelling for at least a month. Whereas a partial response may be defined by a 50% or greater reduction in the swelling of an extremity (e.g., tail in the mouse, arm or leg in a human). Using the findings from the animal studies as a guideline, human trials can be performed using a starting dose selected as that dose where there is less than a grade 3 level toxicity. Dose escalation may be done by 100% increments until drug related grade 2 toxicity is detected and thereafter the dose escalation is stopped.

Of course, the above-described treatment regimes may be altered in accordance with the knowledge gained from clinical trials. Those of skill in the art will be able to take the information disclosed in this specification and optimize treatment regimes based on the clinical trials and animal studies.

### EXAMPLE 2

The following example describes an exemplary procedure for transforming endothelial cells with a FoxC2 gene therapy construct and evaluating gene expression in such cells.

Human primary coronary artery, saphenous vein, human umbilical vein endothelial cells, and microvascular endothelial cells are commercially obtainable, e.g., from Promocell. Lymphatic endothelial cells may be isolated and cultured as described previously in Makinen, T. et al., “Isolated lymphatic endothelial cells transduce growth, survival and migratory signals via the VEGF-C/D receptor VEGFR-3,” Embo J 20, 4762-73. (2001), and are more than 98% pure as determined by staining for lymphatic endothelial markers podoplanin. See also U.S. patent application Ser. No. 10/483,203, filed Jan. 7, 2004 and International Patent Application No. PCT/US02/22164, filed 12 Jul. 2002 and published as WO 03/006104, all incorporated herein by reference.

In another variation, endothelial cells or other cells from FoxC2 mutant mice are used for transformation/transfection studies.

The cells are transfected with a naked plasmid or viral vector constructed for expression of human FoxC2 (recombinant adeno- or lentiviruses) under the control of a constitutively active promoter such as human cytomegalovirus promoter (CMV) (SEQ ID NO: 33) and a polyadenylation sequence.

Northern hybridization, in situ hybridization, and/or antibody assays are used to analyze gene expression in the cells. Elevated levels of FoxC2 mRNA or protein in the cells, relative to untransfected controls, indicates successful transfection and expression of the transgene. Analysis of the expression patterns of other genes regulated by FoxC2 in transfected versus control cells provides an indication of whether the recombinantly expressed FoxC2 is exhibiting transcription factor activity in the cells.

The transgenic cells described in this example also are useful for animal studies and human therapies described above and in subsequent examples.

### EXAMPLE 3

Ex-Vivo Cell Stimulation and Gene Therapy for Lymphedema with FoxC2 Gene-Transfected Cells

Endothelial cells or endothelial precursor cells from a laboratory animal or a human diagnosed with lymphedema distichiasis and a FoxC2 mutation are isolated and transfected with a gene therapy construct to introduce a functional (e.g, a wildtype) FoxC2 gene. The cells optionally are cultured to expand them and then are readministered to the affected animal or human from which they were obtained, in numbers effective numbers to promote improved lymphatic function in vivo. In preferred embodiments, the manipulated cells are autologous cells, but in another variation, the cells are from a donor or derived from stem cells of the same species. The cells are delivered by one or more administrations, e.g., by injection in a region of the body with edema.

In a related variation, the procedure is performed on a genetically affected individual before the onset of lymphedema symptoms, for the purpose of preventing or minimizing such symptoms.

### EXAMPLE 4

Manufacture and Use of Synthetic Valves in Foxc2 Gene Therapy

This example describes the manufacture and use of synthetic venous valves in FoxC2 gene therapy.

A synthetic valve is constructed as described in Pavcnic et al., J. Vasc. Surg., 40:1223-1227 (2004), which is incorporated herein by reference in its entirety. Briefly, the synthetic valve consists of small intestinal submucosa sutured to a square stainless steel stent. The square stent has a short barb at each end to provide anchors for the valve during placement, and the submucosa membrane is slit at the diagonal axis of the stent to create the valve opening.

Endothelial cells transfected with Foxc2, as described in Example 2, are trypsinized, centrifuged, washed and used for seeding of valves at the density of about 100,000 to about 200,000 cells/cm². The synthetic valve is placed in an endothelial cell culture medium for 1-3 days prior to implantation to allow for complete coverage of valve surface with the transfected endothelial cells. Optionally, the synthetic valve is also coated with the FoxC2 gene therapy vector to facilitate localized transfection of endothelial cells in vivo, upon implantation.

The synthetic valves are then implanted purcutaneously into a vein or vessel of a mammalian subject with an over-the-wire 13F or 10F delivery system. Deployment, stability and function of the synthetic valves are studied at immediate venography with contract medium injections peripheral and central to the synthetic valve. Efficacy in the mammalian subject is evaluated with venography and, in test animals, pathologically when sacrificed at 6-12 weeks. Gross pathologic examination will be performed.

Covering venous transplants with endothelial cells is useful to reduce formation of thrombi as well as immunological rejection. Transformation of the cells with FoxC2 and/or Prox1 is expected to reduce the side effect of increased neoinitimal thickening, due to the growth and/or recruitment of fibroblasts or smooth muscle cells, thereby reducing these undesired effects and leading to better acceptance and long term patency of artificial valves.

The protocol is repeated with other synthetic valve designs to demonstrate efficacy of the invention with such valves.

### EXAMPLE 5

Seeding autologous endothelial cells onto prosthetic small diameter prosthetic bypasses has been developed to improve patency, but can cause intimal hyperplasia. The following example describes the seeding of prosthetic grafts with endothelial cells transfected with FoxC2. The protocol is performed as described in Arts et al., Eur. J. Endovasc. Surg., 23:29-38, 2002, incorporated herein by reference.

Briefly, endothelial cells or endothelial precursor cells transfected with Foxc2, as described in Example 2, are trypsinized, centrifuged and washed. Endothelial cells that have not been transfected with FoxC2 serve as a control. Standard ePTFE grafts (expanded polytetrafluoroethylene) vascular grafts are seeded with the endothelial cells or precursor cells transfected with FoxC2, or with untransfected control cells. The grafts and implanted and explanted as described in Verhagen et al., Eur. J. Endovasc. Surg., 15:489-496, 1998. The experiment is repeated with the control.

Patency is determined by angiography and Doppler investigation. A therapeutic benefit is indicated by reduced neointimal thickening in grafts seeded with FoxC2-transfected endothelial cells, compared to controls. The FoxC2 transfected cells are expected to stimulate less growth and/or recruitment of fibroblasts or smooth muscle cells, thereby reducing these undesired effects and leading to better acceptance and long term patency of synthetic grafts.

### EXAMPLE 6

A method of administering a gene therapy construct to a rat model to evaluate venous function has been previously described in Henke et al., J. Immunol., 164: 2131-2141 (2000), which is incorporated herein by reference in its entirety. The following example describes the administration of a FoxC2 gene therapy construct and its effects on blood vessel function and morphology. Chronic venous and possibly lymphatic insufficiency is characterized by extensive inflammatory response, which may play a causative role in the development of the disease. These procedures demonstrate the effects of FoxC2 gene therapy on the modulation of inflammatory response by FOXC2.

Male Sprague Dawley rats, weighing 250-300 g, are anesthetized with an inhalation mixture of isoflurane (1-2%) and oxygen (100%) during the procedure. On day 1, aseptic laparotomy is performed, the inferior vena cava (IVC) is isolated, and major side branches are ligated. An 1.2-cm segment is dissected to allow temporary proximal and distal occlusion with microvascular clips. After occlusion, the IVC is cannulated with a 30-gauge needle catheter, the blood is aspirated, and the IVC is flushed with a dilute heparinized saline solution. After this, 0.15-ml instillation of saline, Ad-CMV-β-gal (or GFP) or AdFOXC2 is performed for 30 minutes. The vector solution or saline is aspirated, the clips are removed to reestablish blood flow, and the midline incision is closed. Two days after infection, animals are sacrificed and infected IVC segments are dissected for further analysis.

To study the effects of FOXC2 on the inflammatory response during venous thrombosis, two days after the transfection, the rats are anesthetized in the same fashion, and a repeat laparotomy is performed. The IVC is then ligated below the renal veins for establishment of thrombosis. This method produces a clot in >95% of animals, and gradual re-establishment of flow usually occurs by dilation of posterior collaterals. Two days after thrombosis induction, the animals are sacrificed. The thrombosed IVC segment is harvested below the renal veins and weighed (milligrams).

The vein is then halved, and the proximal portion is placed in 10% buffered formalin for 24 h, followed by 70% ethanol for subsequent permanent section processing. The lower segment of the vein is snap-frozen in liquid N₂ and kept at −70° C. until homogenized. The infiltration of leukocytes, morphological alterations and expression of cell adhesion molecules such as ICAM-1. ICAM-2 and V-CAM are studied in the control vs AdFOXC2 infected samples using conventional staining techniques (hematoxylin-eosin, immunostaining for lymphocyte markers such as CD45 or using CAM specific antibodies). Snap-frozen portion is used for ELISA based assays of cytokine production, such as IL-1, TNFalpha, IL-10.

The experiments are repeated in other animal models, including animals identified or created with a FoxC2 mutation and animal models with LD phenotypic characteristics. Experiments in dogs, pigs, and primates are contemplated.

### EXAMPLE 7

A method of administering a gene therapy construct extravascularly, e.g., using a device to surround or encapsulate a portion of vessel, has been previously described in WO 98/20027, incorporated herein by reference. The following example describes the use of a collar that is placed around the outside of an artery (e.g., during a bypass procedure) to deliver the FOXC2 gene therapy construct to an arterial wall. The effect of endothelial cell-specific FOXC2 gene transfer on the thickening of the intima is studied using a silicone collar inserted around carotid arteries which acts both as the agent that causes intimal smooth muscle cell growth and as a reservoir for the gene and vector. The model preserves endothelial cell integrity and permits direct extravascular gene transfer without any intravascular manipulation.

Using plasmid/liposome complexes for gene delivery, Moloney murine leukemia virus-derived (MMLV) retroviruses, pseudotyped vesicular stomatitis virusprotein (VSV-G)-containing retroviruses and adenoviruses are delivered into the rabbit carotid artery using a silastic collar applied to the adventitia. The collar is used because 1) it provides a gene delivery reservoir; 2) no intralumenal manipulations are performed and endothelium remains anatomically intact throughout; and 3) installation of the collar induces arterial smooth muscle cell (SMC) proliferation and enhances retroviral gene transfer efficiency where target cell proliferation is required.

Gene Transfer: New Zealand White rabbits (1.8-2.5 kg) are used. The anesthetic is fentanyl-fluanisone (0.3 ml/kg)/midazolam (1 mg/kg)(Yla-Herttuala et al, J. Clin Invest. (1995) 95:2692-2698). A midline neck incision exposes the left carotid artery. A biologically inert 2 cm silastic collar (MediGene Oy, Kuopio, Finland) is positioned around the carotid artery so that it touches the adventitia lightly at either end (Booth et al, Atherosclerosis (1989) 76:257-268). Gene transfer is performed 4-5 days after the collaring operation. For gene transfer, animals are re-anaesthetized. The collar, which has been surgically re-exposed, is gently opened and filled with 500 μl of a FOXC2 gene transfer solution. The incision is closed and arteries are later analyzed for gene transfer efficiency.

Histological Analysis: Collared arteries are carefully removed and divided into three equal parts: the proximal third is immersion-fixed in 4% paraformaldehyde/PBS (pH 7.4) for 15 min, followed by embedding into OCT compound (Miles Scientific, USA). The medial third is immersion-fixed in 4% paraformaldehyde/PBS (pH 7.4) for 4 h, rinsed in 15% sucrose (pH 7.4) for 48 hours and embedded in paraffin. The distal third is embedded in OCT compound and processed for frozen sections. The sections are evaluated via in situ hybridization to determine expression of FoxC2 and of genes regulated by FoxC2 in cells.

In a related experiment, the gene therapy is administered via an adventitial wrap as described in international publication no. WO 99/55315, incorporated herein by reference.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

The references cited herein throughout, to the extent that they provide exemplary procedural or other details supplementary to those set forth herein, are all specifically incorporated herein by reference.

1. A therapeutic or prophylactic method of improving lymphatic function comprising:
administering to a mammalian subject a composition comprising an inhibitor of arterialization of lymphatic vessels,
wherein the subject is selected from: subjects with impaired lymphatic function due to arterialization of lymphatic vessels, and subjects with a genetic risk for developing said impaired function.

2. The method of claim 1, wherein the subject is human.

3. The method of claim 2, wherein the subject has lymphedema from the impaired lymphatic function.

4. The method of claim 2, wherein the subject has lymphedema distichiasis.

5. The method of claim 2, wherein the impaired lymphatic function is diagnosed from abnormal lymphatic drainage, increased number of lymph nodes, lymph flowback, and a lymphatic capillary network that comprises smooth muscle cells associated with lymphatic cells.

6. The method of claim 2, wherein the subject has a Foxc2 mutation genotype.

7. The method of claim 2, wherein the subject has lymphatic vessels characterized by at least one arterialization indicator selected from the group consisting of:
(A) expression of a smooth muscle cell marker in lymphatic capillaries from the subject;
(B) tortuous and distended dermal lymphatic capillaries;
(C) smooth muscle cells associated with lymphatic vessels;
(D) pericytes associated with lymphatic vessels;
(E) lymphatic hyperplasia; and,
(F) expression in lymphatic vessels of a blood vessel basal lamina proteins.

8. The method of claim 2, wherein the subject has lymphatic vessels characterized by absent or dysfunctional lymphatic valves.

9. The method of claim 2, wherein the subject has a genetic risk for developing lymphedema, wherein said risk is diagnosed
from determinations that the subject has one or more of the following: (A) at least one genetic relative with hereditary lymphedema; (B) a Foxc2 mutation genotype; (C) a Foxc1 mutation genotype; (D) distichiasis; (E) smooth muscle cells covering lymphatic capillaries; (F) hyperplastic cutaneous lymphatic vessel density; (G) PDGFR-beta-expressing cells associated with lymphatic vessels; (H) PDGF-B overexpression in lymphatic vessels (I) smooth muscle cell association with lymphatic capillaries; (J) pericyte association with lymphatic capillaries; and (K) expression of blood vessel basal lamina components in lymphatic vessels.

10. The method of claim 9, wherein said risk is further diagnosed from an absence of or dysfunctional lymphatic valves in lymphatic vessels of the subject.

11. The method of claim 9, wherein the subject has at least one genetic relative with hereditary lymphedema and has a Foxc2 mutation genotype.

12. The therapeutic method according to claim 2, wherein the inhibitor of arterialization is administered in an amount effective to reduce edema, reduce pain from edema, increase limb function, increase FOXC2 expression in lymphatic endothelia, reduce PDGF-B expression in lymphatic endothelia, or reduce SMC association with lymphatic capillaries, or combinations thereof.

13. The therapeutic method according to claim 2, wherein the inhibitor of arterialization is administered in an amount effective to reduce lymphatic backflow as judged by lymphoscintigraphy.

14. The prophylactic method according to claim 2, wherein the inhibitor of arterialization is administered in an amount effective to inhibit smooth muscle cell or pericyte association with lymphatic capillaries in the subject.

15-16. (canceled)

17. The method according to claim 1, wherein the inhibitor of arterialization is an inhibitor of PDGFR-beta activity.

18. The method of claim 17, wherein the inhibitor of PDGFR-beta activity is selected from the group consisting of:
(A) inhibitors of PDGFR-beta expression;
(B) inhibitors of PDGF-B stimulation of PDGFR-beta;
(C) inhibitors of PDGF-B expression;
(D) inhibitors of PDGFR-beta signaling; and
(E) combinations thereof.

19. The method of claim 18, wherein the inhibitor of PDGFR-beta activity is an inhibitor of PDGFR-beta expression selected from the group consisting of:
(A) an antisense molecule directed to PDGFR-beta;
(B) an interfering RNA (RNAi) directed to PDGFR-beta;
(C) an aptamer that binds PDGFR-beta RNA;
(D) a ribozyme directed to PDGFR-beta; and
(E) combinations thereof.

20. The method of claim 18, wherein the inhibitor of PDGFR-beta activity is an inhibitor of PDGF-B stimulation of PDGFR-beta selected from the group consisting of:
(A) an antibody substance that binds to the extracellular domain of PDGFR-beta and inhibits PDGF binding;
(B) an antibody substance that binds to PDGF-B and inhibits the PDGF-B from binding or activating PDGFR-beta;
(C) a polypeptide comprising a soluble fragment of PDGFR-beta, wherein the polypeptide and fragment bind PDGF-B;
(D) a fragment of PDGF-B that binds and fails to stimulate PDGFR-beta;
(E) a polypeptide comprising a soluble fragment of PDGFR-alpha, wherein the polypeptide and fragment bind PDGF-B; and
(F) combinations thereof.

21. The method of claim 18, wherein the inhibitor of PDGFR-beta activity is an inhibitor of PDGF-B expression selected from the group consisting of:
(A) an antisense molecule directed to PDGF-B;
(B) interfering RNA (RNAi) directed to PDGF-B;
(C) an aptamer that binds PDGF-B RNA;
(D) a ribozyme directed to PDGF-B; and
(E) combinations thereof.

22. (canceled)

23. The method of claim 18, wherein the inhibitor of PDGFR-beta activity is a tyrosine kinase inhibitor.

24. The method of claim 23, wherein the tyrosine kinase inhibitor is imatinib mesylate.

25. The method of claim 2, wherein the inhibitor of arterialization is administered subcutaneously at a site of edema.

26. A method according to claim 2, further comprising administering to the subject a growth factor product selected from the group consisting of vascular endothelial growth factor C (VEGF-C) protein products, vascular endothelial growth factor D (VEGF-D) protein products, VEGF-C gene therapy products, and VEGF-D gene therapy protein products.

27. A method according to claim 2, further comprising administering to the subject a smooth muscle cell relaxant.

28. A therapeutic or prophylactic method of treating arterialization of lymphatic vessels in a mammalian subject, comprising:
providing isolated lymphatic endothelial cells or lymphatic endothelial progenitor cells;
transforming or transfecting the cells ex vivo with a polynucleotide comprising a nucleotide sequence that encodes an inhibitor of PDGF expression; and
administering the transformed or transfected cells to the mammalian subject.

29. A method according to claim 28, wherein the transformed or transfected cells are administered locally at a site of edema in the subject.

30. A therapeutic or prophylactic method of improving lymphatic function comprising:
administering to a mammalian subject a composition comprising a smooth muscle relaxant;
wherein the subject is identified as having arterialization of lymphatic vessels; and
wherein the smooth muscle relaxant is administered in an amount effective to improve lymphatic function in the subject.

31. The method of claim 30, wherein the composition is administered locally at a site of edema in the subject.

32. A method of screening for an agent to improve lymphatic function in mammalian subjects having arterialization of lymphatic vessels, comprising steps of:
contacting arterialized lymphatic tissue with a test agent, wherein the arterialized lymphatic tissue comprises lymphatic endothelial cells associated with pericytes or smooth muscle cells;
determining if the test agent causes dissociation of lymphatic endothelial cells from pericytes or smooth muscle cells, wherein a test agent that causes the dissociation is selected as an agent to improve lymphatic function.

33. The method of claim 32, wherein the lymphatic tissue is obtained from an organism with a Foxc2 mutation.

34. The method of claim 32, further comprising a step of contacting blood vessels with the test agent, and selecting a test agent that preferentially causes smooth muscle dissociation form lymphatic vessels compared to dissociation of smooth muscle cells from blood vessels.

35. A therapeutic or prophylactic method of improving lymphatic function comprising:
isolating lymphatic endothelial cells or lymphatic endothelial progenitor cells from a subject selected from: subjects with impaired lymphatic function due to arterialization of lymphatic vessels, and subjects with a genetic risk for developing said impaired function;
transforming or transfecting the cells ex vivo with a polynucleotide comprising a nucleic acid sequence that encodes a polypeptide comprising an amino acid sequence at least 90% identical to the Foxc2 amino acid sequence of SEQ ID NO: 2 or a fragment thereof, wherein the polypeptide is expressed in the cells and has Foxc2 transcription factor activity; and
administering the transformed or transfected cells to the mammalian subject, in an amount effective to reduce or prevent lymphatic backflow in a lymphatic vessel, thereby improving lymphatic function.

36-37. (canceled)

38. A method according to claim 35, further comprising a step of coating a synthetic valve with the transformed or transfected cells, wherein the administering comprises implanting the synthetic valve into a vessel in the mammalian subject.

39. A therapeutic or prophylactic method of improving lymphatic function comprising:
administering a composition comprising a polynucleotide to a subject, wherein the subject is selected from: subjects with impaired lymphatic function due to arterialization of lymphatic vessels, and subjects with a genetic risk for developing said impaired function; and wherein the polynucleotide comprises a nucleic acid sequence that encodes a polypeptide comprising an amino acid sequence at least 90% identical to the Foxc2 amino acid sequence of SEQ ID NO: 2 or a fragment thereof, wherein the polypeptide has Foxc2 transcription factor activity.

40. The method of claim 39, wherein said composition is administered locally at a site in need of treatment to improve lymph flow.

41. The method of claim 39, wherein the composition comprises an expression vector that comprises an expression control sequence operatively linked to the polynucleotide.

42. (canceled)

43. The method of claim 39, wherein the polynucleotide further comprises a promoter that promotes expression of the polynucleotide in a mammalian cell.

44. An improvement to a synthetic valve for implantation in a lumen of a blood or lymphatic vessel, said improvement comprising coating a surface of the valve with endothelial cells, wherein the endothelial cells are transformed or transfected with a polynucleotide comprising a nucleotide sequence that encodes a polypeptide that comprises an amino acid sequence at least 90% identical to the Foxc2 amino acid sequence of SEQ ID NO: 2 or a fragment thereof, wherein the polypeptide is expressed in the cells and has Foxc2 transcription factor activity.

45. An isolated endothelial cell or endothelial precursor cell transformed or transfected with a polynucleotide comprising a nucleotide sequence that encodes a polypeptide that comprises an amino acid sequence at least 90% identical to the Foxc2 amino acid sequence of SEQ ID NO: 2 or a fragment thereof, wherein the polypeptide is expressed in the cell and has Foxc2 transcription factor activity.

46. A medical device comprising: a synthetic valve that is implantable in a mammalian vessel; and endothelial cells according to claim 45 on a surface of the synthetic valve.

47. A medical device comprising an endovascular stent that is implantable in a mammalian vessel, and endothelial cells according to claim 43 on a surface of the stent.

48. A therapeutic or prophylactic method of improving venous flow comprising:
isolating venous endothelial cells or venous endothelial progenitor cells from a mammalian subject selected from: subjects with impaired venous flow due to absent or dysfunctional venous valves, and subjects with a genetic risk for developing said impaired flow;
transforming or transfecting the cells ex vivo with a polynucleotide comprising a nucleic acid sequence that encodes a polypeptide comprising an amino acid sequence at least 90% identical to the FOXC2 amino acid sequence of SEQ ID NO: 2 or a fragment thereof, wherein the polypeptide is expressed in the cells and has FOXC2 transcription factor activity; and
administering the transformed or transfected cells to the mammalian subject, in an amount effective to reduce or prevent venous backflow in a blood vessel, thereby improving venous function.

49-51. (canceled)

52. A method according to claim 48, wherein the subject has a FoxC2 mutation genotype.

53. (canceled)

54. A method according to claim 48, wherein a suspension of the cells is administered intravenously into the mammalian subject.

55. A method according to claim 48, further comprising a step of coating a synthetic valve with the transformed or transfected cells, wherein the administering comprises implanting the synthetic valve into a vein in the mammalian subject.

56. A therapeutic or prophylactic method of improving venous flow comprising:
administering a composition comprising a polynucleotide to a subject, wherein the subject is selected from: subjects with impaired venous flow due to the absence of or dysfunctional venous valves, and subjects with a genetic risk for developing said impaired function; and wherein the polynucleotide comprises a nucleic acid sequence that encodes a polypeptide comprising an amino acid sequence at least 90% identical to the FOXC2 amino acid sequence of SEQ ID NO: 2 or a fragment thereof, wherein the polypeptide has Foxc2 transcription factor activity.

57. The method of claim 56, wherein said composition is administered locally at a site in need of treatment to improve venous flow.

58. The method of claim 56, wherein the composition comprises an expression vector that comprises an expression control sequence operatively linked to the polynucleotide.

59. (canceled)

60. The method of claim 56, wherein the polynucleotide further comprises a promoter that promotes expression of the polynucleotide in a mammalian cell.


SEQUENCE LISTING (35 sequences)

SEQ ID NO: 1 - DNA, 1506 bp, Homo sapiens
atgcaggcgcgctactccgtgtccgaccccaacgccctgggagtggtgccctacctgagcgagcagaattactaccgggctgcgggcagctacggcggcatggccagccccatgggcgtctattccggccacccggagcagtacagcgcggggatgggccgctcctacgcgccctaccaccaccaccagcccgcggcgcctaaggacctggtgaagccgccctacagctacatcgcgctcatcaccatggccatccagaacgcgcccgagaagaagatcaccttgaacggcatctaccagttcatcatggaccgcttccccttctaccgggagaacaagcagggctggcagaacagcatccgccacaacctctcgctcaacgagtgcttcgtcaaggtgccccgcgacgacaagaagcccggcaagggcagttactggaccctggacccggactcctacaacatgttcgagaacggcagcttcctgcggcgccggcggcgcttcaaaaagaaggacgtgtccaaggagaaggaggagcgggcccacctcaaggagccgcccccggcggcgtccaagggcgccccggccaccccccacctagcggacgcccccaaggaggccgagaagaaggtggtgatcaagagcgaggcggcgtccccggcgctgccggtcatcaccaaggtggagacgctgagccccgagagcgcgctgcagggcagcccgcgcagcgcggcctccacgcccgccggctcccccgacggttcgctgccggagcaccacgccgcggcgcccaacgggctgcctggcttcagcgtggagaacatcatgaccctgcgaacgtcgccgccgggcggagagctgagcccgggggccggacgcgcgggcctggtggtgccgccgctggcgctgccatacgccgccgcgccgcccgccgcctacggccagccgtgcgctcagggcctggaggccggggccgccgggggctaccagtgcagcatgcgagcgatgagcctgtacaccggggccgagcggccggcgcacatgtgcgtcccgcccgccctggacgaggccctctcggaccacccgagcggccccacgtcgcccctgagcgctctcaacctcgccgccggccaggagggcgcgctcgccgccacgggccaccaccaccagcaccacggccaccaccacccgcaggcgccgccgcccccgccggctccccagccccagccgacgccgcagcccggggccgccgcggcgcaggcggcctcctggtatctcaaccacagcggggacctgaaccacctccccggccacacgttcgcggcccagcagcaaactttccccaacgtgcgggagatgttcaactcccaccggctggggattgagaactcgaccctcggggagtcccaggtgagtggcaatgccagctgccagctgccctacagatccacgccgcctctctatcgccacgcagccccctactcctacgactgcacgaaatactga

SEQ ID NO: 2 - protein, 501 aa, Homo sapiens
MQARYSVSDPNALGVVPYLSEQNYYRAAGSYGGMASPMGVYSGHPEQYSAGMGRSYAPYHHHQPAAPKDLVKPPYSYIALITMAIQNAPEKKITLNGIYQFIMDRFPFYRENKQGWQNSIRHNLSLNECFVKVPRDDKKPGKGSYWTLDPDSYNMFENGSFLRRRRRFKKKDVSKEKEERAHLKEPPPAASKGAPATPHLADAPKEAEKKVVIKSEAASPALPVITKVETLSPESALQGSPRSAASTPAGSPDGSLPEHHAAAPNGLPGFSVENIMTLRTSPPGGELSPGAGRAGLVVPPLALPYAAAPPAAYGQPCAQGLEAGAAGGYQCSMRAMSLYTGAERPAHMCVPPALDEALSDHPSGPTSPLSALNLAAGQEGALAATGHHHQHHGHHHPQAPPPPPAPQPQPTPQPGAAAAQAASWYLNHSGDLNHLPGHTFAAQQQTFPNVREMFNSHRLGIENSTLGESQVSGNASCQLPYRSTPPLYRHAAPYSYDCTKY

SEQ ID NO: 3 - DNA, 2323 bp, Mus musculus
cgcaggcggcgaccggcgtctgggacgcagcatgcaggcgcgttactcggtatcggaccccaacgtgggagtggtaccctatttgagtgagcaaaactactaccgggcggccggcagctacggcggcatggccagccccatgggcgtctactccggccacccggagcagtacggcgccggcatgggccgctcctacgcgccctaccaccaccagcccgcggcgcccaaggacctggtgaagccgccctacagctatatagcgctcatcaccatggcgatccagaacgcgccagagaagaagatcactctgaacggcatctaccagttcatcatggaccgtttccccttctaccgcgagaacaagcagggctggcagaacagcatccgccacaacctgtcactcaatgagtgcttcgtgaaagtgccgcgcgacgacaagaagccgggcaagggcagctactggacgctcgacccggactcctacaacatgttcgagaatggcagcttcctgcggcggcggcggcgcttcaagaagaaggatgtgcccaaggacaaggaggagcgggcccacctcaaggagccgccctcgaccacggccaagggcgctccgacagggaccccggtagctgacgggcccaaggaggccgagaagaaagtcgtggttaagagcgaggcggcgtcccccgcgctgccggtcatcaccaaggtggagacgctgagccccgagggagcgctgcaggccagtccgcgcagcgcatcctccacgcccgcaggttccccagacggctcgctgccggagcaccacgccgcggcgcctaacgggctgcccggcttcagcgtggagaccatcatgacgctgcgcacgtcgcctccgggcggcgatctgagcccagcggccgcgcgcgccggcctggtggtgccaccgctggcactgccatacgccgcagcgccacccgccgcttacacgcagccgtgcgcgcagggcctggaggctgcgggctccgcgggctaccagtgcagtatgcgggctatgagtctgtacaccggggccgagcggcccgcgcacgtgtgcgttccgcccgcgctggacgaggctctgtcggaccacccgagcggccccggctccccgctcggcgccctcaacctcgcagcgggtcaggagggcgcgttgggggcctcgggtcaccaccaccagcatcacggccacctccacccgcaggcgccaccgcccgccccgcagccccctcccgcgccgcagcccgccacccaggccacctcctggtatctgaaccacggcggggacctgagccacctccccggccacacgtttgcaacccaacagcaaactttccccaacgtccgggagatgttcaactcgcaccggctaggactggacaactcgtccctcggggagtcccaggtgagcaatgcgagctgtcagctgccctatcgagctacgccgtccctctaccgccacgcagccccctactcttacgactgcaccaaatactgaggctgtccagtccgctccagccccaggaccgcaccggcttcgcctcctccatgggaaccttcttcgacggagccgcagaaagcgacggaaagcgcccctctctcagaaccaggagcagagagctccgtgcaactcgcaggtaacttatccgcagctcagtttgagatctcagcgagtccctctaagggggatgcagcccagcaaaacgaaatacagattttttttttaattccttcccctacccagatgctgcgcctgctcccttggggcttcatagattagcttatggaccaaacccatagggacccctaatgacttctgtggagattctccacgggcgcaagaggtctctccggataaggtgccttctgtaaacgagtgcggatttgtaaccaggctattttgttcttgcccagagcctttaatataatatttaaagttgtgtccactggataaggtttcgtcttgcccaactgttactgccaaattgaattcaagaaacgtgtgtgggtcttttctccccacgtcaccatgataaaataggtccctccccaaactgtaggtcttttacaaaacaagaaaataatttatttttttgttgttgttggataacgaaattaagtatcggatacttttaatttaggaagtgcatggctttgtacagtagatgccatctggggtattccaaaaacacaccaaaagactttaaaatttcaatctcacctgtgtttgtcttatgtgatctcagtgttgtatttaccttaaaataaacccgtgttgtttttctgcgcaaaaaaaaaaaaaaaaaaa

SEQ ID NO: 4 - protein, 493 aa, Mus musculus
MQARYSVSDPNVGVVPYLSEQNYYRAAGSYGGMASPMGVYSGHPEQYGAGMGRSYAPYHHQPAAPKDLVKPPYSYIALITMAIQNAPEKKITLNGIYQFIMDRFPFYRENKQGWQNSIRHNLSLNECFVKVPRDDKKPGKGSYWTLDPDSYNMFENGSFLRRRRRFKKKDVPKDKEERAHLKEPPSTTAKGAPTGTPVADGPKEAEKKVVVKSEAASPALPVITKVETLSPEGALQASPRSASSTPAGSPDGSLPEHHAAAPNGLPGFSVETIMTLRTSPPGGDLSPAAARAGLVVPPLALPYAAAPPAAYTQPCAQGLEAAGSAGYQCSMRAMSLYTGAERPAHVCVPPALDEALSDHPSGPGSPLGALNLAAGQEGALGASGHHHQHHGHLHPQAPPPAPQPPPAPQPATQATSWYLNHGGDLSHLPGHTFATQQQTFPNVREMFNSHRLGLDNSSLGESQVSNASCQLPYRATPSLYRHAAPYSYDCTKY

SEQ ID NO: 5 - DNA, 1997 bp, Homo sapiens
  CDS (352)..(1608)
cccgccccgcctctccaaaaagctacaccgacgcggaccgcggcggcgtcctccctcgccctcgcttcacctcgcgggctccgaatgcggggagctcggatgtccggtttcctgtgaggcttttacctgacacccgccgcctttccccggcactggctgggagggcgccctgcaaagttgggaacgcggagccccggacccgctcccgccgcctccggctcgcccagggggggtcgccgggaggagcccgggggagagggaccaggaggggcccgcggcctcgcaggggcgcccgcgcccccacccctgcccccgccagcggaccggtcccccacccccggtccttccaccatgcacttgctgggcttcttctctgtggcgtgttctctgctcgccgctgcgctgctcccgggtcctcgcgaggcgcccgccgccgccgccgccttcgagtccggactcgacctctcggacgcggagcccgacgcgggcgaggccacggcttatgcaagcaaagatctggaggagcagttacggtctgtgtccagtgtagatgaactcatgactgtactctacccagaatattggaaaatgtacaagtgtcagctaaggaaaggaggctggcaacataacagagaacaggccaacctcaactcaaggacagaagagactataaaatttgctgcagcacattataatacagagatcttgaaaagtattgataatgagtggagaaagactcaatgcatgccacgggaggtgtgtatagatgtggggaaggagtttggagtcgcgacaaacaccttctttaaacctccatgtgtgtccgtctacagatgtgggggttgctgcaatagtgaggggctgcagtgcatgaacaccagcacgagctacctcagcaagacgttatttgaaattacagtgcctctctctcaaggccccaaaccagtaacaatcagttttgccaatcacacttcctgccgatgcatgtctaaactggatgtttacagacaagttcattccattattagacgttccctgccagcaacactaccacagtgtcaggcagcgaacaagacctgccccaccaattacatgtggaataatcacatctgcagatgcctggctcaggaagattttatgttttcctcggatgctggagatgactcaacagatggattccatgacatctgtggaccaaacaaggagctggatgaagagacctgtcagtgtgtctgcagagcggggcttcggcctgccagctgtggaccccacaaagaactagacagaaactcatgccagtgtgtctgtaaaaacaaactcttccccagccaatgtggggccaaccgagaatttgatgaaaacacatgccagtgtgtatgtaaaagaacctgccccagaaatcaacccctaaatcctggaaaatgtgcctgtgaatgtacagaaagtccacagaaatgcttgttaaaaggaaagaagttccaccaccaaacatgcagctgttacagacggccatgtacgaaccgccagaaggcttgtgagccaggattttcatatagtgaagaagtgtgtcgttgtgtcccttcatattggaaaagaccacaaatgagctaagattgtactgttttccagttcatcgattttctattatggaaaactgtgttgccacagtagaactgtctgtgaacagagagacccttgtgggtccatgctaacaaagacaaaagtctgtctttcctgaaccatgtggataactttacagaaatggactggagctcatctgcaaaaggcctcttgtaaagactggttttctgccaatgaccaaacagccaagattttcctcttgtgatttctttaaaagaatgactatataatttatttccactaaaaatattgtttctgcattcatttttatagcaacaacaattggtaaaactcactgtgatcaatatttttatatcatgcaaaatatgtttaaaataaaatgaaaattgtattat

SEQ ID NO: 6 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 7 - DNA, 2029 bp, Homo sapiens
  CDS (411)..(1472)
gttgggttccagctttctgtagctgtaagcattggtggccacaccacctccttacaaagcaactagaacctgcggcatacattggagagatttttttaattttctggacatgaagtaaatttagagtgctttctaatttcaggtagaagacatgtccaccttctgattatttttggagaacattttgatttttttcatctctctctccccacccctaagattgtgcaaaaaaagcgtaccttgcctaattgaaataatttcattggattttgatcagaactgattatttggttttctgtgtgaagttttgaggtttcaaactttccttctggagaatgccttttgaaacaattttctctagctgcctgatgtcaactgcttagtaatcagtggatattgaaatattcaaaatgtacagagagtgggtagtggtgaatgttttcatgatgttgtacgtccagctggtgcagggctccagtaatgaacatggaccagtgaagcgatcatctcagtccacattggaacgatctgaacagcagatcagggctgcttctagtttggaggaactacttcgaattactcactctgaggactggaagctgtggagatgcaggctgaggctcaaaagttttaccagtatggactctcgctcagcatcccatcggtccactaggtttgcggcaactttctatgacattgaaacactaaaagttatagatgaagaatggcaaagaactcagtgcagccctagagaaacgtgcgtggaggtggccagtgagctggggaagagtaccaacacattcttcaagcccccttgtgtgaacgtgttccgatgtggtggctgttgcaatgaagagagccttatctgtatgaacaccagcacctcgtacatttccaaacagctctttgagatatcagtgcctttgacatcagtacctgaattagtgcctgttaaagttgccaatcatacaggttgtaagtgcttgccaacagccccccgccatccatactcaattatcagaagatccatccagatccctgaagaagatcgctgttcccattccaagaaactctgtcctattgacatgctatgggatagcaacaaatgtaaatgtgttttgcaggaggaaaatccacttgctggaacagaagaccactctcatctccaggaaccagctctctgtgggccacacatgatgtttgacgaagatcgttgcgagtgtgtctgtaaaacaccatgtcccaaagatctaatccagcaccccaaaaactgcagttgctttgagtgcaaagaaagtctggagacctgctgccagaagcacaagctatttcacccagacacctgcagctgtgaggacagatgcccctttcataccagaccatgtgcaagtggcaaaacagcatgtgcaaagcattgccgctttccaaaggagaaaagggctgcccaggggccccacagccgaaagaatccttgattcagcgttccaagttccccatccctgtcatttttaacagcatgctgctttgccaagttgctgtcactgtttttttcccaggtgttaaaaaaaaaatccattttacacagcaccacagtgaatccagaccaaccttccattcacaccagctaaggagtccctggttcattgatggatgtcttctagctgcagatgcctctgcgcaccaaggaatggagaggaggggacccatgtaatccttttgtttagttttgtttttgttttttggtgaatgagaaaggtgtgctggtcatggaatggcaggtgtcatatgactgattactcagagcagatgaggaaaactgtagtctctgagtcctttgctaatcgcaactcttgtgaattattctgattcttttttatgcagaatttgattcgtatgatcagtactgactttctgattactgtccagcttatagtcttccagtttaatgaactaccatctgatgtttcatatttaagtgtatttaaagaaaataaacaccattattcaagccaaaaaaaaaaaaaaaaaa

SEQ ID NO: 8 - protein, 354 aa, Homo sapiens
MYREWVVVNVFMMLYVQLVQGSSNEHGPVKRSSQSTLERSEQQIRAASSLEELLRITHSEDWKLWRCRLRLKSFTSMDSRSASHRSTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 9 - DNA, 4795 bp, Homo sapiens
  CDS (20)..(4111)
ccacgcgcagcggccggagatgcagcggggcgccgcgctgtgcctgcgactgtggctctgcctgggactcctggacggcctggtgagtggctactccatgacccccccgaccttgaacatcacggaggagtcacacgtcatcgacaccggtgacagcctgtccatctcctgcaggggacagcaccccctcgagtgggcttggccaggagctcaggaggcgccagccaccggagacaaggacagcgaggacacgggggtggtgcgagactgcgagggcacagacgccaggccctactgcaaggtgttgctgctgcacgaggtacatgccaacgacacaggcagctacgtctgctactacaagtacatcaaggcacgcatcgagggcaccacggccgccagctcctacgtgttcgtgagagactttgagcagccattcatcaacaagcctgacacgctcttggtcaacaggaaggacgccatgtgggtgccctgtctggtgtccatccccggcctcaatgtcacgctgcgctcgcaaagctcggtgctgtggccagacgggcaggaggtggtgtgggatgaccggcggggcatgctcgtgtccacgccactgctgcacgatgccctgtacctgcagtgcgagaccacctggggagaccaggacttcctttccaaccccttcctggtgcacatcacaggcaacgagctctatgacatccagctgttgcccaggaagtcgctggagctgctggtaggggagaagctggtcctgaactgcaccgtgtgggctgagtttaactcaggtgtcacctttgactgggactacccagggaagcaggcagagcggggtaagtgggtgcccgagcgacgctcccagcagacccacacagaactctccagcatcctgaccatccacaacgtcagccagcacgacctgggctcgtatgtgtgcaaggccaacaacggcatccagcgatttcgggagagcaccgaggtcattgtgcatgaaaatcccttcatcagcgtcgagtggctcaaaggacccatcctggaggccacggcaggagacgagctggtgaagctgcccgtgaagctggcagcgtaccccccgcccgagttccagtggtacaaggatggaaaggcactgtccgggcgccacagtccacatgccctggtgctcaaggaggtgacagaggccagcacaggcacctacaccctcgccctgtggaactccgctgctggcctgaggcgcaacatcagcctggagctggtggtgaatgtgcccccccagatacatgagaaggaggcctcctcccccagcatctactcgcgtcacagccgccaggccctcacctgcacggcctacggggtgcccctgcctctcagcatccagtggcactggcggccctggacaccctgcaagatgtttgcccagcgtagtctccggcggcggcagcagcaagacctcatgccacagtgccgtgactggagggcggtgaccacgcaggatgccgtgaaccccatcgagagcctggacacctggaccgagtttgtggagggaaagaataagactgtgagcaagctggtgatccagaatgccaacgtgtctgccatgtacaagtgtgtggtctccaacaaggtgggccaggatgagcggctcatctacttctatgtgaccaccatccccgacggcttcaccatcgaatccaagccatccgaggagctactagagggccagccggtgctcctgagctgccaagccgacagctacaagtacgagcatctgcgctggtaccgcctcaacctgtccacgctgcacgatgcgcacgggaacccgcttctgctcgactgcaagaacgtgcatctgttcgccacccctctggccgccagcctggaggaggtggcacctggggcgcgccacgccacgctcagcctgagtatcccccgcgtcgcgcccgagcacgagggccactatgtgtgcgaagtgcaagaccggcgcagccatgacaagcactgccacaagaagtacctgtcggtgcaggccctggaagcccctcggctcacgcagaacttgaccgacctcctggtgaacgtgagcgactcgctggagatgcagtgcttggtggccggagcgcacgcgcccagcatcgtgtggtacaaagacgagaggctgctggaggaaaagtctggagtcgacttggcggactccaaccagaagctgagcatccagcgcgtgcgcgaggaggatgcgggacgctatctgtgcagcgtgtgcaacgccaagggctgcgtcaactcctccgccagcgtggccgtggaaggctccgaggataagggcagcatggagatcgtgatccttgtcggtaccggcgtcatcgctgtcttcttctgggtcctcctcctcctcatcttctgtaacatgaggaggccggcccacgcagacatcaagacgggctacctgtccatcatcatggaccccggggaggtgcctctggaggagcaatgcgaatacctgtcctacgatgccagccagtgggaattcccccgagagcggctgcacctggggagagtgctcggctacggcgccttcgggaaggtggtggaagcctccgctttcggcatccacaagggcagcagctgtgacaccgtggccgtgaaaatgctgaaagagggcgccacggccagcgagcaccgcgcgctgatgtcggagctcaagatcctcattcacatcggcaaccacctcaacgtggtcaacctcctcggggcgtgcaccaagccgcagggccccctcatggtgatcgtggagttctgcaagtacggcaacctctccaacttcctgcgcgccaagcgggacgccttcagcccctgcgcggagaagtctcccgagcagcgcggacgcttccgcgccatggtggagctcgccaggctggatcggaggcggccggggagcagcgacagggtcctcttcgcgcggttctcgaagaccgagggcggagcgaggcgggcttctccagaccaagaagctgaggacctgtggctgagcccgctgaccatggaagatcttgtctgctacagcttccaggtggccagagggatggagttcctggcttcccgaaagtgcatccacagagacctggctgctcggaacattctgctgtcggaaagcgacgtggtgaagatctgtgactttggccttgcccgggacatctacaaagaccctgactacgtccgcaagggcagtgcccggctgcccctgaagtggatggcccctgaaagcatcttcgacaaggtgtacaccacgcagagtgacgtgtggtcctttggggtgcttctctgggagatcttctctctgggggcctccccgtaccctggggtgcagatcaatgaggagttctgccagcggctgagagacggcacaaggatgagggccccggagctggccactcccgccatacgccgcatcatgctgaactgctggtccggagaccccaaggcgagacctgcattctcggagctggtggagatcctgggggacctgctccagggcaggggcctgcaagaggaagaggaggtctgcatggccccgcgcagctctcagagctcagaagagggcagcttctcgcaggtgtccaccatggccctacacatcgcccaggctgacgctgaggacagcccgccaagcctgcagcgccacagcctggccgccaggtattacaactgggtgtcctttcccgggtgcctggccagaggggctgagacccgtggttcctccaggatgaagacatttgaggaattccccatgaccccaacgacctacaaaggctctgtggacaaccagacagacagtgggatggtgctggcctcggaggagtttgagcagatagagagcaggcatagacaagaaagcggcttcagctgtaaaggacctggccagaatgtggctgtgaccagggcacaccctgactcccaagggaggcggcggcggcctgagcggggggcccgaggaggccaggtgttttacaacagcgagtatggggagctgtcggagccaagcgaggaggaccactgctccccgtctgcccgcgtgactttcttcacagacaacagctactaagcagcatcggacaagacccccagcacttgggggttcaggcccggcagggcgggcagagggctggaggcccaggctgggaactcatctggttgaactctggtggcacaggagtgtcctcttccctctctgcagacttcccagctaggaagagcaggactccaggcccaaggctcccggaattccgtcaccacgactggccagggcacgctccagctgccccggcccctccccctgagattcagatgtcatttagttcagcatccgcaggtgctggtcccggggccagcacttccatgggaatgtctctttggcgacctcctttcatcacactgggtggtggcctggtccctgttttcccacgaggaatctgtgggtctgggagtcacacagtgttggaggttaaggcatacgagagcagaggtctcccaaacgccctttcctcctcaggcacacagctactctccccacgagggctggctggcctcacccacccctgcacagttgaagggaggggctgtgtttccatctcaaagaaggcatttgcagggtcctcttctgggcctgaccaaacagccaactagcccctggggtggccaccagtatgacagtattatacgctggcaacacagaggcagcccgcacacctgcgcctgggtgttgagagccatcctgcaagtctttttc

SEQ ID NO: 10 - protein, 1363 aa, Homo sapiens
MQRGAALCLRLWLCLGLLDGLVSGYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHENPFISVEWLKGPILEATAGDELVKLPVKLAAYPPPEFQWYKDGKALSGRHSPHALVLKEVTEASTGTYTLALWNSAAGLRRNISLELVVNVPPQIHEKEASSPSIYSRHSRQALTCTAYGVPLPLSIQWHWRPWTPCKMFAQRSLRRRQQQDLMPQCRDWRAVTTQDAVNPIESLDTWTEFVEGKNKTVSKLVIQNANVSAMYKCVVSNKVGQDERLIYFYVTTIPDGFTIESKPSEELLEGQPVLLSCQADSYKYEHLRWYRLNLSTLHDAHGNPLLLDCKNVHLFATPLAASLEEVAPGARHATLSLSIPRVAPEHEGHYVCEVQDRRSHDKHCHKKYLSVQALEAPRLTQNLTDLLVNVSDSLEMQCLVAGAHAPSIVWYKDERLLEEKSGVDLADSNQKLSIQRVREEDAGRYLCSVCNAKGCVNSSASVAVEGSEDKGSMEIVILVGTGVIAVFFWVLLLLIFCNMRRPAHADIKTGYLSIIMDPGEVPLEEQCEYLSYDASQWEFPRERLHLGRVLGYGAFGKVVEASAFGIHKGSSCDTVAVKMLKEGATASEHRALMSELKILIHIGNHLNVVNLLGACTKPQGPLMVIVEFCKYGNLSNFLRAKRDAFSPCAEKSPEQRGRFRAMVELARLDRRRPGSSDRVLFARFSKTEGGARRASPDQEAEDLWLSPLTMEDLVCYSFQVARGMEFLASRKCIHRDLAARNILLSESDVVKICDFGLARDIYKDPDYVRKGSARLPLKWMAPESIFDKVYTTQSDVWSFGVLLWEIFSLGASPYPGVQINEEFCQRLRDGTRMRAPELATPAIRRIMLNCWSGDPKARPAFSELVEILGDLLQGRGLQEEEEVCMAPRSSQSSEEGSFSQVSTMALHIAQADAEDSPPSLQRHSLAARYYNWVSFPGCLARGAETRGSSRMKTFEEFPMTPTTYKGSVDNQTDSGMVLASEEFEQIESRHRQESGFSCKGPGQNVAVTRAHPDSQGRRRRPERGARGGQVFYNSEYGELSEPSEEDHCSPSARVTFFTDNSY

SEQ ID NO: 11 - DNA, 4195 bp, Homo sapiens
  CDS (20)..(3913)
ccacgcgcagcggccggagatgcagcggggcgccgcgctgtgcctgcgactgtggctctgcctgggactcctggacggcctggtgagtggctactccatgacccccccgaccttgaacatcacggaggagtcacacgtcatcgacaccggtgacagcctgtccatctcctgcaggggacagcaccccctcgagtgggcttggccaggagctcaggaggcgccagccaccggagacaaggacagcgaggacacgggggtggtgcgagactgcgagggcacagacgccaggccctactgcaaggtgttgctgctgcacgaggtacatgccaacgacacaggcagctacgtctgctactacaagtacatcaaggcacgcatcgagggcaccacggccgccagctcctacgtgttcgtgagagactttgagcagccattcatcaacaagcctgacacgctcttggtcaacaggaaggacgccatgtgggtgccctgtctggtgtccatccccggcctcaatgtcacgctgcgctcgcaaagctcggtgctgtggccagacgggcaggaggtggtgtgggatgaccggcggggcatgctcgtgtccacgccactgctgcacgatgccctgtacctgcagtgcgagaccacctggggagaccaggacttcctttccaaccccttcctggtgcacatcacaggcaacgagctctatgacatccagctgttgcccaggaagtcgctggagctgctggtaggggagaagctggtcctgaactgcaccgtgtgggctgagtttaactcaggtgtcacctttgactgggactacccagggaagcaggcagagcggggtaagtgggtgcccgagcgacgctcccagcagacccacacagaactctccagcatcctgaccatccacaacgtcagccagcacgacctgggctcgtatgtgtgcaaggccaacaacggcatccagcgatttcgggagagcaccgaggtcattgtgcatgaaaatcccttcatcagcgtcgagtggctcaaaggacccatcctggaggccacggcaggagacgagctggtgaagctgcccgtgaagctggcagcgtaccccccgcccgagttccagtggtacaaggatggaaaggcactgtccgggcgccacagtccacatgccctggtgctcaaggaggtgacagaggccagcacaggcacctacaccctcgccctgtggaactccgctgctggcctgaggcgcaacatcagcctggagctggtggtgaatgtgcccccccagatacatgagaaggaggcctcctcccccagcatctactcgcgtcacagccgccaggccctcacctgcacggcctacggggtgcccctgcctctcagcatccagtggcactggcggccctggacaccctgcaagatgtttgcccagcgtagtctccggcggcggcagcagcaagacctcatgccacagtgccgtgactggagggcggtgaccacgcaggatgccgtgaaccccatcgagagcctggacacctggaccgagtttgtggagggaaagaataagactgtgagcaagctggtgatccagaatgccaacgtgtctgccatgtacaagtgtgtggtctccaacaaggtgggccaggatgagcggctcatctacttctatgtgaccaccatccccgacggcttcaccatcgaatccaagccatccgaggagctactagagggccagccggtgctcctgagctgccaagccgacagctacaagtacgagcatctgcgctggtaccgcctcaacctgtccacgctgcacgatgcgcacgggaacccgcttctgctcgactgcaagaacgtgcatctgttcgccacccctctggccgccagcctggaggaggtggcacctggggcgcgccacgccacgctcagcctgagtatcccccgcgtcgcgcccgagcacgagggccactatgtgtgcgaagtgcaagaccggcgcagccatgacaagcactgccacaagaagtacctgtcggtgcaggccctggaagcccctcggctcacgcagaacttgaccgacctcctggtgaacgtgagcgactcgctggagatgcagtgcttggtggccggagcgcacgcgcccagcatcgtgtggtacaaagacgagaggctgctggaggaaaagtctggagtcgacttggcggactccaaccagaagctgagcatccagcgcgtgcgcgaggaggatgcgggacgctatctgtgcagcgtgtgcaacgccaagggctgcgtcaactcctccgccagcgtggccgtggaaggctccgaggataagggcagcatggagatcgtgatccttgtcggtaccggcgtcatcgctgtcttcttctgggtcctcctcctcctcatcttctgtaacatgaggaggccggcccacgcagacatcaagacgggctacctgtccatcatcatggaccccggggaggtgcctctggaggagcaatgcgaatacctgtcctacgatgccagccagtgggaattcccccgagagcggctgcacctggggagagtgctcggctacggcgccttcgggaaggtggtggaagcctccgctttcggcatccacaagggcagcagctgtgacaccgtggccgtgaaaatgctgaaagagggcgccacggccagcgagcaccgcgcgctgatgtcggagctcaagatcctcattcacatcggcaaccacctcaacgtggtcaacctcctcggggcgtgcaccaagccgcagggccccctcatggtgatcgtggagttctgcaagtacggcaacctctccaacttcctgcgcgccaagcgggacgccttcagcccctgcgcggagaagtctcccgagcagcgcggacgcttccgcgccatggtggagctcgccaggctggatcggaggcggccggggagcagcgacagggtcctcttcgcgcggttctcgaagaccgagggcggagcgaggcgggcttctccagaccaagaagctgaggacctgtggctgagcccgctgaccatggaagatcttgtctgctacagcttccaggtggccagagggatggagttcctggcttcccgaaagtgcatccacagagacctggctgctcggaacattctgctgtcggaaagcgacgtggtgaagatctgtgactttggccttgcccgggacatctacaaagaccctgactacgtccgcaagggcagtgcccggctgcccctgaagtggatggcccctgaaagcatcttcgacaaggtgtacaccacgcagagtgacgtgtggtcctttggggtgcttctctgggagatcttctctctgggggcctccccgtaccctggggtgcagatcaatgaggagttctgccagcggctgagagacggcacaaggatgagggccccggagctggccactcccgccatacgccgcatcatgctgaactgctggtccggagaccccaaggcgagacctgcattctcggagctggtggagatcctgggggacctgctccagggcaggggcctgcaagaggaagaggaggtctgcatggccccgcgcagctctcagagctcagaagagggcagcttctcgcaggtgtccaccatggccctacacatcgcccaggctgacgctgaggacagcccgccaagcctgcagcgccacagcctggccgccaggtattacaactgggtgtcctttcccgggtgcctggccagaggggctgagacccgtggttcctccaggatgaagacatttgaggaattccccatgaccccaacgacctacaaaggctctgtggacaaccagacagacagtgggatggtgctggcctcggaggagtttgagcagatagagagcaggcatagacaagaaagcggcttcaggtagctgaagcagagagagagaaggcagcatacgtcagcattttcttctctgcacttataagaaagatcaaagactttaagactttcgctatttcttctactgctatctactacaaacttcaaagaggaaccaggaggacaagaggagcatgaaagtggacaaggagtgtgaccactgaagcaccacagggaaggggttaggcctccggatgactgcgggcaggcctggataatatccagcctcccacaagaagctggtggagcagagtgttccctgactcct

SEQ ID NO: 12 - protein, 1298 aa, Homo sapiens
MQRGAALCLRLWLCLGLLDGLVSGYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHENPFISVEWLKGPILEATAGDELVKLPVKLAAYPPPEFQWYKDGKALSGRHSPHALVLKEVTEASTGTYTLALWNSAAGLRRNISLELVVNVPPQIHEKEASSPSIYSRHSRQALTCTAYGVPLPLSIQWHWRPWTPCKMFAQRSLRRRQQQDLMPQCRDWRAVTTQDAVNPIESLDTWTEFVEGKNKTVSKLVIQNANVSAMYKCVVSNKVGQDERLIYFYVTTIPDGFTIESKPSEELLEGQPVLLSCQADSYKYEHLRWYRLNLSTLHDAHGNPLLLDCKNVHLFATPLAASLEEVAPGARHATLSLSIPRVAPEHEGHYVCEVQDRRSHDKHCHKKYLSVQALEAPRLTQNLTDLLVNVSDSLEMQCLVAGAHAPSIVWYKDERLLEEKSGVDLADSNQKLSIQRVREEDAGRYLCSVCNAKGCVNSSASVAVEGSEDKGSMEIVILVGTGVIAVFFWVLLLLIFCNMRRPAHADIKTGYLSIIMDPGEVPLEEQCEYLSYDASQWEFPRERLHLGRVLGYGAFGKVVEASAFGIHKGSSCDTVAVKMLKEGATASEHRALMSELKILIHIGNHLNVVNLLGACTKPQGPLMVIVEFCKYGNLSNFLRAKRDAFSPCAEKSPEQRGRFRAMVELARLDRRRPGSSDRVLFARFSKTEGGARRASPDQEAEDLWLSPLTMEDLVCYSFQVARGMEFLASRKCIHRDLAARNILLSESDVVKICDFGLARDIYKDPDYVRKGSARLPLKWMAPESIFDKVYTTQSDVWSFGVLLWEIFSLGASPYPGVQINEEFCQRLRDGTRMRAPELATPAIRRIMLNCWSGDPKARPAFSELVEILGDLLQGRGLQEEEEVCMAPRSSQSSEEGSFSQVSTMALHIAQADAEDSPPSLQRHSLAARYYNWVSFPGCLARGAETRGSSRMKTFEEFPMTPTTYKGSVDNQTDSGMVLASEEFEQIESRHRQESGFR

SEQ ID NO: 13 - DNA, 6375 bp, Homo sapiens
  CDS (129)..(3398)
  misc_feature (4476)..(4476): n is a, c, g, or t
ttggagctacagggagagaaacagaggaggagactgcaagagatcattggaggccgtgggcacgctctttactccatgtgtgggacattcattgcggaataacatcggaggagaagtttcccagagctatggggacttcccatccggcgttcctggtcttaggctgtcttctcacagggctgagcctaatcctctgccagctttcattaccctctatccttccaaatgaaaatgaaaaggttgtgcagctgaattcatccttttctctgagatgctttggggagagtgaagtgagctggcagtaccccatgtctgaagaagagagctccgatgtggaaatcagaaatgaagaaaacaacagcggcctttttgtgacggtcttggaagtgagcagtgcctcggcggcccacacagggttgtacacttgctattacaaccacactcagacagaagagaatgagcttgaaggcaggcacatttacatctatgtgccagacccagatgtagcctttgtacctctaggaatgacggattatttagtcatcgtggaggatgatgattctgccattataccttgtcgcacaactgatcccgagactcctgtaaccttacacaacagtgagggggtggtacctgcctcctacgacagcagacagggctttaatgggaccttcactgtagggccctatatctgtgaggccaccgtcaaaggaaagaagttccagaccatcccatttaatgtttatgctttaaaagcaacatcagagctggatctagaaatggaagctcttaaaaccgtgtataagtcaggggaaacgattgtggtcacctgtgctgtttttaacaatgaggtggttgaccttcaatggacttaccctggagaagtgaaaggcaaaggcatcacaatgctggaagaaatcaaagtcccatccatcaaattggtgtacactttgacggtccccgaggccacggtgaaagacagtggagattacgaatgtgctgcccgccaggctaccagggaggtcaaagaaatgaagaaagtcactatttctgtccatgagaaaggtttcattgaaatcaaacccaccttcagccagttggaagctgtcaacctgcatgaagtcaaacattttgttgtagaggtgcgggcctacccacctcccaggatatcctggctgaaaaacaatctgactctgattgaaaatctcactgagatcaccactgatgtggaaaagattcaggaaataaggtatcgaagcaaattaaagctgatccgtgctaaggaagaagacagtggccattatactattgtagctcaaaatgaagatgctgtgaagagctatacttttgaactgttaactcaagttccttcatccattctggacttggtcgatgatcaccatggctcaactgggggacagacggtgaggtgcacagctgaaggcacgccgcttcctgatattgagtggatgatatgcaaagatattaagaaatgtaataatgaaacttcctggactattttggccaacaatgtctcaaacatcatcacggagatccactcccgagacaggagtaccgtggagggccgtgtgactttcgccaaagtggaggagaccatcgccgtgcgatgcctggctaagaatctccttggagctgagaaccgagagctgaagctggtggctcccaccctgcgttctgaactcacggtggctgctgcagtcctggtgctgttggtgattgtgatcatctcacttattgtcctggttgtcatttggaaacagaaaccgaggtatgaaattcgctggagggtcattgaatcaatcagcccagatggacatgaatatatttatgtggacccgatgcagctgccttatgactcaagatgggagtttccaagagatggactagtgcttggtcgggtcttggggtctggagcgtttgggaaggtggttgaaggaacagcctatggattaagccggtcccaacctgtcatgaaagttgcagtgaagatgctaaaacccacggccagatccagtgaaaaacaagctctcatgtctgaactgaagataatgactcacctggggccacatttgaacattgtaaacttgctgggagcctgcaccaagtcaggccccatttacatcatcacagagtattgcttctatggagatttggtcaactatttgcataagaatagggatagcttcctgagccaccacccagagaagccaaagaaagagctggatatctttggattgaaccctgctgatgaaagcacacggagctatgttattttatcttttgaaaacaatggtgactacatggacatgaagcaggctgatactacacagtatgtccccatgctagaaaggaaagaggtttctaaatattccgacatccagagatcactctatgatcgtccagcctcatataagaagaaatctatgttagactcagaagtcaaaaacctcctttcagatgataactcagaaggccttactttattggatttgttgagcttcacctatcaagttgcccgaggaatggagtttttggcttcaaaaaattgtgtccaccgtgatctggctgctcgcaacgttctcctggcacaaggaaaaattgtgaagatctgtgactttggcctggccagagacatcatgcatgattcgaactatgtgtcgaaaggcagtacctttctgcccgtgaagtggatggctcctgagagcatctttgacaacctctacaccacactgagtgatgtctggtcttatggcattctgctctgggagatcttttcccttggtggcaccccttaccccggcatgatggtggattctactttctacaataagatcaagagtgggtaccggatggccaagcctgaccacgctaccagtgaagtctacgagatcatggtgaaatgctggaacagtgagccggagaagagaccctccttttaccacctgagtgagattgtggagaatctgctgcctggacaatataaaaagagttatgaaaaaattcacctggacttcctgaagagtgaccatcctgctgtggcacgcatgcgtgtggactcagacaatgcatacattggtgtcacctacaaaaacgaggaagacaagctgaaggactgggagggtggtctggatgagcagagactgagcgctgacagtggctacatcattcctctgcctgacattgaccctgtccctgaggaggaggacctgggcaagaggaacagacacagctcgcagacctctgaagagagtgccattgagacgggttccagcagttccaccttcatcaagagagaggacgagaccattgaagacatcgacatgatggacgacatcggcatagactcttcagacctggtggaagacagcttcctgtaactggcggattcgaggggttccttccacttctggggccacctctggatcccgttcagaaaaccactttattgcaatgcggaggttgagaggaggacttggttgatgtttaaagagaagttcccagccaagggcctcggggagcctttctaaatatgaatgaatgggatattttgaaatgaactttgtcagtgttgcctcttgcaatgcctcagtagcatctcagtggtgtgtgaagtttggagatagatggataagggaataataggccacagaaggtgaactttctgcttcaaggacattggtgagagtccaacagacacaatttatactgcgacagaacttcagcattgtaattatgtaaataactctaaccacggctgtgtttagattgtattaactatcttctttggacttctgaagagaccactcaatccatccatgtacttccctcttgaaacctgatgtcagctgctgttgaactttttaaagaagtgcatgaaaaaccatttttgaccttaaaaggtactggtactatagcattttgctatcttttttagtgttaaagagataaagaataataattaaccaaccttgtttaatagatttgggtcatttagaagcctgacaactcattttcatattgtaatctatgtttataatactactactgttatcagtaatgctaaatgtgtaataatgtaacatgatttccctccacacaaagcacaatttaaaaacaatccttactaagtaggtgatgagtttgacagtttttgacatttatattaaataacatgtttctctataaagtatggtaatagctttagtgaattaaatttagttgagcatagagaacaaagtaaaagtagtgttgtccaggaagtcagaatttttaactgtactgaataggttccccaatccatcgtattaaaaaacaattaactgccctctgaaataatgggattagaaacaaacaaaactcttaagtcctaaaagttctcaatgtagaggcataaacctgtgctgaacataacttctcatgtatattacccaatggaaaatataatgatcagcgcanaaagactggatttgcagaagttnttttttttttttcttcttgcctgatgaaagctttggcgaccccaatatatgtattttttgaatctatgaacctgaaaagggtcacaaaggatgcccagacatcagcctccttctttcaccccttaccccaaagagaaagagtttgaaactcgagaccataaagatattctttagtggaggctggaagtgcattagcctgatcctcagttctcaaatgtgtgtggcagccaggtagactagtacctgggtttccatccttgagattctgaagtatgaagtctgagggaaaccagagtctgtatttttctaaactccctggctgttctgatcggccaggtttcggaaacactgacttaggtttcaggaagttgccatgggaaacaaataatttgaactttggaacagggttcttaagttggtgcgtccttcggatgataaatttaggaaccgaagtccaatcactgtaaattacggtagatcgatcgttaacgctggaattaaattgaaaggtcagaatcgactccgactctttcgatttcaaaccaaaactgtccaaaaggttttcatttctacgatgaagggtgacataccccctctaacttgaaaggggcagagggcagaagagcggagggtgaggtatggggcggttcctttccgtacatgtttttaatacgttaagtcacaaggttcagagacacattggtcgagtcacaaaaccaccttttttgtaaaattcaaaatgactattaaactccaatctaccctcctacttaacagtgtagataggtgtgacagtttgtccaaccacacccaagtaaccgtaagaaacgttatgacgaattaacgactatggtatacttactttgtacccgacactaatgacgttagtgacacgatagccgtctactacgaaaccttctacgtcttcgttattatttcatgaactgatggatgaccacattagagttacgttcggggttgaaagaataggttgaaaaagtatcattcacgcttctgactcggtctaaccggttaatttttcttttggactgatccaagacatctcggttaatctgaactttatgcaaacacaaagatcttagtgtcgagttcgtaagacaaatagcgagtgagagggaacatgtcggaataaaacaaccacgaaacgtaaaactataacgacactcggaacgtactgtagtactccggcctactttgaagagtcaggtcgtcaaaggtcaggattgtttacgagggtggacttaaacatatactgacgtaaacacccacacacacacaaaagtcgtttaaggtctaaacaaaggaaaaccggaggacgtttcagaggtcttcttttaaacggttagaaaggatgaaagataaaaatactactgttagtttcggccggactctttgtgataaacactgaaaaatttgctaatcactacaggaattttacaccagacggttagacatgttttaccaggataaaaacacttctccctgtattctattttactacaatatgtagttatacatatatacataaagatatatctgaacctcttatgacggttttgtaaatactgttcgacatagtgacggaagcaaatataaaaaaattgacactattaggggtgtccgtgtaattgacaacgtgaaaacttacaggttttaaatataaaatctttattatttttctttctatgaatgtacaagggttttgttaccacaccacttacacactctttttgattgaactatcccagatggttatgttttacataatgcttacggggacaagtacaaaaacaaaattttgcacatttacttctagaaatataaagttatttactatatattaaatttccttaag

SEQ ID NO: 14 - protein, 1089 aa, Homo sapiens
MGTSHPAFLVLGCLLTGLSLILCQLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKATSELDLEMEALKTVYKSGETIVVTCAVFNNEVVDLQWTYPGEVKGKGITMLEEIKVPSIKLVYTLTVPEATVKDSGDYECAARQATREVKEMKKVTISVHEKGFIEIKPTFSQLEAVNLHEVKHFVVEVRAYPPPRISWLKNNLTLIENLTEITTDVEKIQEIRYRSKLKLIRAKEEDSGHYTIVAQNEDAVKSYTFELLTQVPSSILDLVDDHHGSTGGQTVRCTAEGTPLPDIEWMICKDIKKCNNETSWTILANNVSNIITEIHSRDRSTVEGRVTFAKVEETIAVRCLAKNLLGAENRELKLVAPTLRSELTVAAAVLVLLVIVIISLIVLVVIWKQKPRYEIRWRVIESISPDGHEYIYVDPMQLPYDSRWEFPRDGLVLGRVLGSGAFGKVVEGTAYGLSRSQPVMKVAVKMLKPTARSSEKQALMSELKIMTHLGPHLNIVNLLGACTKSGPIYIITEYCFYGDLVNYLHKNRDSFLSHHPEKPKKELDIFGLNPADESTRSYVILSFENNGDYMDMKQADTTQYVPMLERKEVSKYSDIQRSLYDRPASYKKKSMLDSEVKNLLSDDNSEGLTLLDLLSFTYQVARGMEFLASKNCVHRDLAARNVLLAQGKIVKICDFGLARDIMHDSNYVSKGSTFLPVKWMAPESIFDNLYTTLSDVWSYGILLWEIFSLGGTPYPGMMVDSTFYNKIKSGYRMAKPDHATSEVYEIMVKCWNSEPEKRPSFYHLSEIVENLLPGQYKKSYEKIHLDFLKSDHPAVARMRVDSDNAYIGVTYKNEEDKLKDWEGGLDEQRLSADSGYIIPLPDIDPVPEEEDLGKRNRHSSQTSEESAIETGSSSSTFIKREDETIEDIDMMDDIGIDSSDLVEDSFL

SEQ ID NO: 15 - DNA, 5427 bp, Homo sapiens
  CDS (187)..(3507)
tgttctcctgagccttcaggagcctgcaccagtcctgcctgtccttctactcagctgttacccactctgggaccagcagtctttctgataactgggagagggcagtaaggaggacttcctggagggggtgactgtccagagcctggaactgtgcccacaccagaagccatcagcagcaaggacaccatgcggcttccgggtgcgatgccagctctggccctcaaaggcgagctgctgttgctgtctctcctgttacttctggaaccacagatctctcagggcctggtcgtcacacccccggggccagagcttgtcctcaatgtctccagcaccttcgttctgacctgctcgggttcagctccggtggtgtgggaacggatgtcccaggagcccccacaggaaatggccaaggcccaggatggcaccttctccagcgtgctcacactgaccaacctcactgggctagacacgggagaatacttttgcacccacaatgactcccgtggactggagaccgatgagcggaaacggctctacatctttgtgccagatcccaccgtgggcttcctccctaatgatgccgaggaactattcatctttctcacggaaataactgagatcaccattccatgccgagtaacagacccacagctggtggtgacactgcacgagaagaaaggggacgttgcactgcctgtcccctatgatcaccaacgtggcttttctggtatctttgaggacagaagctacatctgcaaaaccaccattggggacagggaggtggattctgatgcctactatgtctacagactccaggtgtcatccatcaacgtctctgtgaacgcagtgcagactgtggtccgccagggtgagaacatcaccctcatgtgcattgtgatcgggaatgatgtggtcaacttcgagtggacatacccccgcaaagaaagtgggcggctggtggagccggtgactgacttcctcttggatatgccttaccacatccgctccatcctgcacatccccagtgccgagttagaagactcggggacctacacctgcaatgtgacggagagtgtgaatgaccatcaggatgaaaaggccatcaacatcaccgtggttgagagcggctacgtgcggctcctgggagaggtgggcacactacaatttgctgagctgcatcggagccggacactgcaggtagtgttcgaggcctacccaccgcccactgtcctgtggttcaaagacaaccgcaccctgggcgactccagcgctggcgaaatcgccctgtccacgcgcaacgtgtcggagacccggtatgtgtcagagctgacactggttcgcgtgaaggtggcagaggctggccactacaccatgcgggccttccatgaggatgctgaggtccagctctccttccagctacagatcaatgtccctgtccgagtgctggagctaagtgagagccaccctgacagtggggaacagacagtccgctgtcgtggccggggcatgccgcagccgaacatcatctggtctgcctgcagagacctcaaaaggtgtccacgtgagctgccgcccacgctgctggggaacagttccgaagaggagagccagctggagactaacgtgacgtactgggaggaggagcaggagtttgaggtggtgagcacactgcgtctgcagcacgtggatcggccactgtcggtgcgctgcacgctgcgcaacgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttacgagatccgatggaaggtgattgagtctgtgagctctgacggccatgagtacatctacgtggaccccatgcagctgccctatgactccacgtgggagctgccgcgggaccagcttgtgctgggacgcaccctcggctctggggcctttgggcaggtggtggaggccacagctcatggtctgagccattctcaggccacgatgaaagtggccgtcaagatgcttaaatccacagcccgcagcagtgagaagcaagcccttatgtcggagctgaagatcatgagtcaccttgggccccacctgaacgtggtcaacctgttgggggcctgcaccaaaggaggacccatctatatcatcactgagtactgccgctacggagacctggtggactacctgcaccgcaacaaacacaccttcctgcagcaccactccgacaagcgccgcccgcccagcgcggagctctacagcaatgctctgcccgttgggctccccctgcccagccatgtgtccttgaccggggagagcgacggtggctacatggacatgagcaaggacgagtcggtggactatgtgcccatgctggacatgaaaggagacgtcaaatatgcagacatcgagtcctccaactacatggccccttacgataactacgttccctctgcccctgagaggacctgccgagcaactttgatcaacgagtctccagtgctaagctacatggacctcgtgggcttcagctaccaggtggccaatggcatggagtttctggcctccaagaactgcgtccacagagacctggcggctaggaacgtgctcatctgtgaaggcaagctggtcaagatctgtgactttggcctggctcgagacatcatgcgggactcgaattacatctccaaaggcagcacctttttgcctttaaagtggatggctccggagagcatcttcaacagcctctacaccaccctgagcgacgtgtggtccttcgggatcctgctctgggagatcttcaccttgggtggcaccccttacccagagctgcccatgaacgagcagttctacaatgccatcaaacggggttaccgcatggcccagcctgcccatgcctccgacgagatctatgagatcatgcagaagtgctgggaagagaagtttgagattcggccccccttctcccagctggtgctgcttctcgagagactgttgggcgaaggttacaaaaagaagtaccagcaggtggatgaggagtttctgaggagtgaccacccagccatccttcggtcccaggcccgcttgcctgggttccatggcctccgatctcccctggacaccagctccgtcctctatactgccgtgcagcccaatgagggtgacaacgactatatcatccccctgcctgaccccaaacctgaggttgctgacgagggcccactggagggttcccccagcctagccagctccaccctgaatgaagtcaacacctcctcaaccatctcctgtgacagccccctggagccccaggacgaaccagagccagagccccagcttgagctccaggtggagccggagccggagctggaacagttgccggattcggggtgccctgcgcctcgggcggaagcagaggatagcttcctgtagggggctggcccctaccctgccctgcctgaagctcccccgctgccagcacccagcatctcctggcctggcctggccgggcttcctgtcagccaggctgcccttatcagctgtccccttctggaagctttctgctcctgacgtgttgtgccccaaaccctggggctggcttaggaggcaagaaaactgcaggggccgtgaccagccctctgcctccagggaggccaactgactctgagccagggttcccccagggaactcagttttcccatatgtaagatgggaaagttaggcttgatgacccagaatctaggattctctccctggctgacaggtggggagaccgaatccctccctgggaagattcttggagttactgaggtggtaaattaacttttttctgttcagccagctacccctcaaggaatcatagctctctcctcgcacttttatccacccaggagctagggaagagaccctagcctccctggctgctggctgagctagggcctagccttgagcagtgttgcctcatccagaagaaagccagtctcctccctatgatgccagtccctgcgttccctggcccgagctggtctggggccattaggcagcctaattaatgctggaggctgagccaagtacaggacacccccagcctgcagcccttgcccagggcacttggagcacacgcagccatagcaagtgcctgtgtccctgtccttcaggcccatcagtcctggggctttttctttatcaccctcagtcttaatccatccaccagagtctagaaggccagacgggccccgcatctgtgatgagaatgtaaatgtgccagtgtggagtggccacgtgtgtgtgccagatatggccctggctctgcattggacctgctatgaggctttggaggaatccctcaccctctctgggcctcagtttccccttcaaaaaatgaataagtcggacttattaactctgagtgccttgccagcactaacattctagagtatccaggtggttgcacatttgtccagatgaagcaaggccatataccctaaacttccatcctgggggtcagctgggctcctgggagattccagatcacacatcacactctggggactcaggaaccatgccccttccccaggcccccagcaagtctcaagaacacagctgcacaggccttgacttagagtgacagccggtgtcctggaaagcccccagcagctgccccagggacatgggaagaccacgggacctctttcactacccacgatgacctccgggggtatcctgggcaaaagggacaaagagggcaaatgagatcacctcctgcagcccaccactccagcacctgtgccgaggtctgcgtcgaagacagaatggacagtgaggacagttatgtcttgtaaaagacaagaagcttcagatgggtaccccaagaaggatgtgagaggtgggcgctttggaggtttgcccctcacccaccagctgccccatccctgaggcagcgctccatgggggtatggttttgtcactgcccagacctagcagtgacatctcattgtccccagcccagtgggcattggaggtgccaggggagtcagggttgtagccaagacgcccccgcacggggagggttgggaagggggtgcaggaagctcaacccctctgggcaccaaccctgcattgcaggttggcaccttacttccctgggatcccagagttggtccaaggagggagagtgggttctcaatacggtaccaaagatataatcacctaggtttacaaatatttttaggactcacgttaactcacatttatacagcagaaatgctattttgtatgctgttaagtttttctatctgtgtacttttttttaagggaaagattttaatattaaacctggtgcttctcactcac

SEQ ID NO: 16 - protein, 1106 aa, Homo sapiens [sequence omitted]

SEQ ID NO: 17 - DNA, 2305 bp, Homo sapiens
  CDS (404)..(991)
ttcttggggctgatgtccgcaaatatgcagaattaccggccgggtcgctcctgaagccagcgcggggagcgagcgcggcggcggccagcaccgggaacgcaccgaggaagaagcccagcccccgccctccgccccttccgtccccaccccctacccggcggcccaggaggctccccggctgcggcgcgcactccctgtttctcctcctcctggctggcgctgcctgcctctccgcactcactgctcgccgggcgccgtccgccagctccgtgctccccgcgccaccctcctccgggccgcgctccctaagggatggtactgaatttcgccgccacaggagaccggctggagcgcccgccccgcgcctcgcctctcctccgagcagccagcgcctcgggacgcgatgaggaccttggcttgcctgctgctcctcggctgcggatacctcgcccatgttctggccgaggaagccgagatcccccgcgaggtgatcgagaggctggcccgcagtcagatccacagcatccgggacctccagcgactcctggagatagactccgtagggagtgaggattctttggacaccagcctgagagctcacggggtccacgccactaagcatgtgcccgagaagcggcccctgcccattcggaggaagagaagcatcgaggaagctgtccccgctgtctgcaagaccaggacggtcatttacgagattcctcggagtcaggtcgaccccacgtccgccaacttcctgatctggcccccgtgcgtggaggtgaaacgctgcaccggctgctgcaacacgagcagtgtcaagtgccagccctcccgcgtccaccaccgcagcgtcaaggtggccaaggtggaatacgtcaggaagaagccaaaattaaaagaagtccaggtgaggttagaggagcatttggagtgcgcctgcgcgaccacaagcctgaatccggattatcgggaagaggacacggatgtgaggtgaggatgagccgcagccctttcctgggacatggatgtacatggcgtgttacattcctgaacctactatgtacggtgctttattgccagtgtgcggtctttgttctcctccgtgaaaaactgtgtccgagaacactcgggagaacaaagagacagtgcacatttgtttaatgtgacatcaaagcaagtattgtagcactcggtgaagcagtaagaagcttccttgtcaaaaagagagagagagagagagagagagaaaacaaaaccacaaatgacaaaaacaaaacggactcacaaaaatatctaaactcgatgagatggagggtcgccccgtgggatggaagtgcagaggtctcagcagactggatttctgtccgggtggtcacaggtgcttttttgccgaggatgcagagcctgctttgggaacgactccagaggggtgctggtgggctctgcagggcccgcaggaagcaggaatgtcttggaaaccgccacgcgaactttagaaaccacacctcctcgctgtagtatttaagcccatacagaaaccttcctgagagccttaagtggtttttttttttgtttttgttttgttttttttttttttgttttttttttttttttttttttttacaccataaagtgattattaagcttccttttactctttggctagctttttttttttttttttttttttttttttttaattatctcttggatgacatttacaccgataacacacaggctgctgtaactgtcaggacagtgcgacggtatttttcctagcaagatgcaaactaatgagatgtattaaaataaacatggtatacctacctatgcatcatttcctaaatgtttctggctttgtgtttctcccttaccctgctttatttgttaatttaagccattttgaaagaactatgcgtcaaccaatcgtacgccgtccctgcggcacctgccccagagcccgtttgtggctgagtgacaacttgttccccgcagtgcacacctagaatgctgtgttcccacgcggcacgtgagatgcattgccgcttctgtctgtgttgttggtgtgccctggtgccgtggtggcggtcactccctctgctgccagtgtttggacagaacccaaattctttatttttggtaagatattgtgctttacctgtattaacagaaatgtgtgtgtgtggtttgtttttttgtaaaggtgaagtttgtatgtttacctaatattacctgttttgtatacctgagagcctgctatgttcttcttttgttgatccaaaattaaaaaaaaaataccaccaac

SEQ ID NO: 18 - protein, 196 aa, Homo sapiens
MRTLACLLLLGCGYLAHVLAEEAEIPREVIERLARSQIHSIRDLQRLLEIDSVGSEDSLDTSLRAHGVHATKHVPEKRPLPIRRKRSIEEAVPAVCKTRTVIYEIPRSQVDPTSANFLIWPPCVEVKRCTGCCNTSSVKCQPSRVHHRSVKVAKVEYVRKKPKLKEVQVRLEEHLECACATTSLNPDYREEDTDVR

SEQ ID NO: 19 - DNA, 2137 bp, Homo sapiens
  CDS (983)..(1705)
ccctgcctgcctccctgcgcacccgcagcctcccccgctgcctccctagggctcccctccggccgccagcgcccatttttcattccctagatagagatactttgcgcgcacacacatacatacgcgcgcaaaaaggaaaaaaaaaaaaaaaagcccaccctccagcctcgctgcaaagagaaaaccggagcagccgcagctcgcagctcgcagcccgcagcccgcagaggacgcccagagcggcgagcgggcgggcagacggaccgacggactcgcgccgcgtccacctgtcggccgggcccagccgagcgcgcagcgggcacgccgcgcgcgcggagcagccgtgcccgccgcccgggcccgccgccagggcgcacacgctcccgcccccctacccggcccgggcgggagtttgcacctctccctgcccgggtgctcgagctgccgttgcaaagccaactttggaaaaagttttttgggggagacttgggccttgaggtgcccagctccgcgctttccgattttgggggcctttccagaaaatgttgcaaaaaagctaagccggcgggcagaggaaaacgcctgtagccggcgagtgaagacgaaccatcgactgccgtgttccttttcctcttggaggttggagtcccctgggcgcccccacacggctagacgcctcggctggttcgcgacgcagccccccggccgtggatgctgcactcgggctcgggatccgcccaggtagcggcctcggacccaggtcctgcgcccaggtcctcccctgccccccagcgacggagccggggccgggggcggcggcgccgggggcatgcgggtgagccgcggctgcagaggcctgagcgcctgatcgccgcggacccgagccgagcccacccccctccccagccccccaccctggccgcgggggcggcgcgctcgatctacgcgttcggggccccgcggggccgggcccggagtcggcatgaatcgctgctgggcgctcttcctgtctctctgctgctacctgcgtctggtcagcgccgagggggaccccattcccgaggagctttatgagatgctgagtgaccactcgatccgctcctttgatgatctccaacgcctgctgcacggagaccccggagaggaagatggggccgagttggacctgaacatgacccgctcccactctggaggcgagctggagagcttggctcgtggaagaaggagcctgggttccctgaccattgctgagccggccatgatcgccgagtgcaagacgcgcaccgaggtgttcgagatctcccggcgcctcatagaccgcaccaacgccaacttcctggtgtggccgccctgtgtggaggtgcagcgctgctccggctgctgcaacaaccgcaacgtgcagtgccgccccacccaggtgcagctgcgacctgtccaggtgagaaagatcgagattgtgcggaagaagccaatctttaagaaggccacggtgacgctggaagaccacctggcatgcaagtgtgagacagtggcagctgcacggcctgtgacccgaagcccggggggttcccaggagcagcgagccaaaacgccccaaactcgggtgaccattcggacggtgcgagtccgccggccccccaagggcaagcaccggaaattcaagcacacgcatgacaagacggcactgaaggagacccttggagcctaggggcatcggcaggagagtgtgtgggcagggttatttaatatggtatttgctgtattgcccccatggggccttggagtagataatattgtttccctcgtccgtctgtctcgatgcctgattcggacggccaatggtgcctcccccacccctccacgtgtccgtccacccttccatcagcgggtctcctcccagcggcctccggctcttgcccagcagctcaagaagaaaaagaaggactgaactccatcgccatcttcttcccttaactccaagaacttgggataagagtgtgagagagactgatggggtcgctctttgggggaaacgggttccttcccctgcacctggcctgggccacacctgagcgctgtggactgtcctgaggagccctgaggacctctcagcatagcctgcctgatccctgaaccc

SEQ ID NO: 20 - protein, 241 aa, Homo sapiens
MNRCWALFLSLCCYLRLVSAEGDPIPEELYEMLSDHSIRSFDDLQRLLHGDPGEEDGAELDLNMTRSHSGGELESLARGRRSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA

SEQ ID NO: 21 - DNA, 2108 bp, Homo sapiens
  misc_feature (2002)..(2002): n is a, c, g, or t
ccccgccgtgagtgagctctcaccccagtcagccaaatgagcctcttcgggcttctcctggtgacatctgccctggccggccagagacgagggactcaggcggaatccaacctgagtagtaaattccagttttccagcaacaaggaacagaacggagtacaagatcctcagcatgagagaattattactgtgtctactaatggaagtattcacagcccaaggtttcctcatacttatccaagaaatacggtcttggtatggagattagtagcagtagaggaaaatgtatggatacaacttacgtttgatgaaagatttgggcttgaagacccagaagatgacatatgcaagtatgattttgtagaagttgaggaacccagtgatggaactatattagggcgctggtgtggttctggtactgtaccaggaaaacagatttctaaaggaaatcaaattaggataagatttgtatctgatgaatattttccttctgaaccagggttctgcatccactacaacattgtcatgccacaattcacagaagctgtgagtccttcagtgctacccccttcagctttgccactggacctgcttaataatgctataactgcctttagtaccttggaagaccttattcgatatcttgaaccagagagatggcagttggacttagaagatctatataggccaacttggcaacttcttggcaaggcttttgtttttggaagaaaatccagagtggtggatctgaaccttctaacagaggaggtaagattatacagctgcacacctcgtaacttctcagtgtccataagggaagaactaaagagaaccgataccattttctggccaggttgtctcctggttaaacgctgtggtgggaactgtgcctgttgtctccacaattgcaatgaatgtcaatgtgtcccaagcaaagttactaaaaaataccacgaggtccttcagttgagaccaaagaccggtgtcaggggattgcacaaatcactcaccgacgtggccctggagcaccatgaggagtgtgactgtgtgtgcagagggagcacaggaggatagccgcatcaccaccagcagctcttgcccagagctgtgcagtgcagtggctgattctattagagaacgtatgcgttatctccatccttaatctcagttgtttgcttcaaggacctttcatcttcaggatttacagtgcattctgaaagaggagacatcaaacagaattaggagttgtgcaacagctcttttgagaggaggcctaaaggacaggagaaaaggtcttcaatcgtggaaagaaaattaaatgttgtattaaatagatcaccagctagtttcagagttaccatgtacgtattccactagctgggttctgtatttcagttctttcgatacggcttagggtaatgtcagtacaggaaaaaaactgtgcaagtgagcacctgattccgttgccttgcttaactctaaagctccatgtcctgggcctaaaatcgtataaaatctggatttttttttttttttttgctcatattcacatatgtaaaccagaacattctatgtactacaaacctggtttttaaaaaggaactatgttgctatgaattaaacttgtgtcrtgctgataggacagactggatttttcatatttcttattaaaatttctgccatttagaagaagagaactacattcatggtttggaagagataaacctgaaaagaagagtggccttatcttcactttatcgataagtcagtttatttgtttcattgtgtacatttttatattctccttttgacattataactgttggcttttctaatcttgttaaatatatctatttttaccaaaggtatttaatattcttttttatgacaacttagatcaactatttttagcttggtaaatttttctaaacacaattgttatagccagaggaacaaagatggatataaaaatattgttgccctggacaaaaatacatgtatntccatcccggaatggtgctagagttggattaaacctgcattttaaaaaacctgaattgggaanggaanttggtaaggttggccaaancttttttgaaaataattaa

SEQ ID NO: 22 - protein, 345 aa, Homo sapiens [sequence omitted]

SEQ ID NO: 23 - DNA, 2253 bp, Homo sapiens
cgctcggaaagttcagcatgcaggaagtttggggagagctcggcgattagcacagcgacccgggccagcgcagggcgagcgcaggcggcgagagcgcagggcggcgcggcgtcggtcccgggagcagaacccggctttttcttggagcgacgctgtctctagtcgctgatcccaaatgcaccggctcatctttgtctacactctaatctgcgcaaacttttgcagctgtcgggacacttctgcaaccccgcagagcgcatccatcaaagctttgcgcaacgccaacctcaggcgagatgagagcaatcacctcacagacttgtaccgaagagatgagaccatccaggtgaaaggaaacggctacgtgcagagtcctagattcccgaacagctaccccaggaacctgctcctgacatggcggcttcactctcaggagaatacacggatacagctagtgtttgacaatcagtttggattagaggaagcagaaaatgatatctgtaggtatgattttgtggaagttgaagatatatccgaaaccagtaccattattagaggacgatggtgtggacacaaggaagttcctccaaggataaaatcaagaacgaaccaaattaaaatcacattcaagtccgatgactactttgtggctaaacctggattcaagatttattattctttgctggaagatttccaacccgcagcagcttcagagaccaactgggaatctgtcacaagctctatttcaggggtatcctataactctccatcagtaacggatcccactctgattgcggatgctctggacaaaaaaattgcagaatttgatacagtggaagatctgctcaagtacttcaatccagagtcatggcaagaagatcttgagaatatgtatctggacacccctcggtatcgaggcaggtcataccatgaccggaagtcaaaagttgacctggataggctcaatgatgatgccaagcgttacagttgcactcccaggaattactcggtcaatataagagaagagctgaagttggccaatgtggtcttctttccacgttgcctcctcgtgcagcgctgtggaggaaattgtggctgtggaactgtcaactggaggtcctgcacatgcaattcagggaaaaccgtgaaaaagtatcatgaggtattacagtttgagcctggccacatcaagaggaggggtagagctaagaccatggctctagttgacatccagttggatcaccatgaacgatgcgattgtatctgcagctcaagaccacctcgataagagaatgtgcacatccttacattaagcctgaaagaacctttagtttaaggagggtgagataagagacccttttcctaccagcaaccaaacttactactagcctgcaatgcaatgaacacaagtggttgctgagtctcagccttgctttgttaatgccatggcaagtagaaaggtatatcatcaacttctatacctaagaatataggattgcatttaataatagtgtttgaggttatatatgcacaaacacacacagaaatatattcatgtctatgtgtatatagatcaaatgttttttttggtatatataaccaggtacaccagagcttacatatgtttgagttagactcttaaaatcctttgccaaaataagggatggtcaaatatatgaaacatgtctttagaaaatttaggagataaatttatttttaaattttgaaacacaaaacaattttgaatcttgctctcttaaagaaagcatcttgtatattaaaaatcaaaagatgaggctttcttacatatacatcttagttgattattaaaaaaggaaaaaggtttccagagaaaaggccaatacctaagcattttttccatgagaagcactgcatacttacctatgtggactgtaataacctgtctccaaaaccatgccataataatataagtgctttagaaattaaatcattgtgttttttatgcattttgctgaggcatccttattcatttaacacctatctcaaaaacttacttagaaggttttttattatagtcctacaaaagacaatgtataagctgtaacagaattttgaattgtttttctttgcaaaacccctccacaaaagcaaatcctttcaagaatggcatgggcattctgtatgaacctttccagatggtgttcagtgaaagatgtgggtagttgagaacttaaaaagtgaacattgaaacatcgacgtaactggaaaccg

SEQ ID NO: 24 - protein, 116 aa, Homo sapiens
GRKSRVVDLNLLTEEVRLYSCTPRNFSVSIREELKRTDTIFWPGCLLVKRCGGNCACCLHNCNECQCVPSKVTKKYHEVLQLRPKTGVRGLHKSLTDVALEHHEECDCVCRGSTGG

SEQ ID NO: 25 - DNA, 54 bp, Artificial sequence
  Synthetic primer
gatccttaagtaagtaaacaaacaagatccttaagtaagtaaacaaacaagatc

SEQ ID NO: 26 - DNA, 53 bp, Artificial sequence
  Synthetic primer
gatcttgtttgtttacttacttaaggatcttgtttgtttacttacttaaggat

SEQ ID NO: 27 - DNA, 1571 bp, Homo sapiens
ctgcaccaagctggccctgcacggctgcaagggaggctcctgtggacaggccaggcaggtgggcctcaggaggtgcctccaggcggccagtgggcctgaggccccagcaagggctagggtccatctccagtcccaggacacagcagcggccaccatggccacgcctgggctccagcagcatcagcccccaggaccggggaggcacaggtggcccccaccacccggaggagcagctcctgcccctgtccgggggatgactgattctcctccgccaggccacccagaggagaaggccaccccgcctggaggcacaggccatgaggggctctcaggaggtgctgctgatgtggcttctggtgttggcagtgggcggcacagagcacgcctaccggcccggccgtagggtgtgtgctgtccgggctcacggggaccctgtctccgagtcgttcgtgcagcgtgtgtaccagcccttcctcaccacctgcgacgggcaccgggcctgcagcacctaccgaaccatctataggaccgcctaccgccgcagccctgggctggcccctgccaggcctcgctacgcgtgctgccccggctggaagaggaccagcgggcttcctggggcctgtggagcagcaatatgccagccgccatgccggaacggagggagctgtgtccagcctggccgctgccgctgccctgcaggatggcggggtgacacttgccagtcagatgtggatgaatgcagtgctaggaggggcggctgtccccagcgctgcgtcaacaccgccggcagttactggtgccagtgttgggaggggcacagcctgtctgcagacggtacactctgtgtgcccaagggagggccccccagggtggcccccaacccgacaggagtggacagtgcaatgaaggaagaagtgcagaggctgcagtccagggtggacctgctggaggagaagctgcagctggtgctggccccactgcacagcctggcctcgcaggcactggagcatgggctcccggaccccggcagcctcctggtgcactccttccagcagctcggccgcatcgactccctgagcgagcagatttccttcctggaggagcagctggggtcctgctcctgcaagaaagactcgtgactgcccagcgccccaggctggactgagcccctcacgccgccctgcagcccccatgcccctgcccaacatgctgggggtccagaagccacctcggggtgactgagcggaaggccaggcagggccttcctcctcttcctcctccccttcctcgggaggctccccagaccctggcatgggatgggctgggatcttctctgtgaatccacccctggctacccccaccctggctaccccaacggcatcccaaggccaggtgggccctcagctgagggaaggtacgagctccctgctggagcctgggacccatggcacaggccaggcagcccggaggctgggtggggcctcagtgggggctgctgcctgacccccagcacaataaaaatgaaacgtgaaaaaaaaaaaaaaaaaaaaaaaaaaaaaaaaaaaaaaaaa

SEQ ID NO: 28 - protein, 273 aa, Homo sapiens
MRGSQEVLLMWLLVLAVGGTEHAYRPGRRVCAVRAHGDPVSESFVQRVYQPFLTTCDGHRACSTYRTIYRTAYRRSPGLAPARPRYACCPGWKRTSGLPGACGAAICQPPCRNGGSCVQPGRCRCPAGWRGDTCQSDVDECSARRGGCPQRCVNTAGSYWCQCWEGHSLSADGTLCVPKGGPPRVAPNPTGVDSAMKEEVQRLQSRVDLLEEKLQLVLAPLHSLASQALEHGLPDPGSLLVHSFQQLGRIDSLSEQISFLEEQLGSCSCKKDS

SEQ ID NO: 29 - DNA, 1569 bp, Homo sapiens
ccaagctggccctgcacggctgcaagggaggctcctgtggacaggccaggcaggtgggcctcaggaggtgcctccaggcggccagtgggcctgaggccccagcaagggctagggtccatctccagtcccaggacacagcagcggccaccatggccacgcctgggctccagcagcatcagcagcccccaggaccggggaggcacaggtggcccccaccacccggaggagcagctcctgcccctgtccgggggatgactgattctcctccgccaggccacccagaggagaaggccaccccgcctggaggcacaggccatgaggggctctcaggaggtgctgctgatgtggcttctggtgttggcagtgggcggcacagagcacgcctaccggcccggccgtagggtgtgtgctgtccgggctcacggggaccctgtctccgagtcgttcgtgcagcgtgtgtaccagcccttcctcaccacctgcgacgggcaccgggcctgcagcacctaccgaaccatctataggaccgcctaccgccgcagccctgggctggcccctgccaggcctcgctacgcgtgctgccccggctggaagaggaccagcgggcttcctggggcctgtggagcagcaatatgccagccgccatgccggaacggagggagctgtgtccagcctggccgctgccgctgccctgcaggatggcggggtgacacttgccagtcagatgtggatgaatgcagtgctaggaggggcggctgtccccagcgctgcgtcaacaccgccggcagttactggtgccagtgttgggaggggcacagcctgtctgcagacggtacactctgtgtgcccaagggagggccccccagggtggcccccaacccgacaggagtggacagtgcaatgaaggaagaagtgcagaggctgcagtccagggtggacctgctggaggagaagctgcagctggtgctggccccactgcacagcctggcctcgcaggcactggagcatgggctcccggaccccggcagcctcctggtgcactccttccagcagctcggccgcatcgactccctgagcgagcagatttccttcctggaggagcagctggggtcctgctcctgcaagaaagactcgtgactgcccagcgccccaggctggactgagcccctcacgccgccctgcagcccccatgcccctgcccaacatgctgggggtccagaagccacctcggggtgactgagcggaaggccaggcagggccttcctcctcttcctcctccccttcctcgggaggctccccagaccctggcatgggatgggctgggatcttctctgtgaatccacccctggctacccccaccctggctaccccaacggcatcccaaggccaggtgggccctcagctgagggaaggtacgagctccctgctggagcctgggacccatggcacaggccaggcagcccggaggctgggtggggcctcagtgggggctgctgcctgacccccagcacaataaaaatgaaacgtgaaaaaaaaaaaaaaaaaaaaaaaaaaaaaaaaaaaaaaaaa

SEQ ID NO: 30 - protein, 273 aa, Homo sapiens; identical to SEQ ID NO: 28

SEQ ID NO: 31 - DNA, 14896 bp, Homo sapiens
cagcggtgcgagctccaggcccatgcactgaggaggcggaaacaaggggagcccccagagctccatcaagccccctccaaaggctcccctacccggtccacgccccccaccccccctccccgcctcctcccaattgtgcatttttgcagccggaggcggctccgagatggggctgtgagcttcgcccggggagggggaaagagcagcgaggagtgaagcgggggggtggggtgaagggtttggatttcggggcagggggcgcacccccgtcagcaggccctccccaaggggctcggaactctacctcttcacccacgcccctggtgcgctttgccgaaggaaagaataagaacagagaaggaggagggggaaaggaggaaaagggggaccccccaactggggggggtgaaggagagaagtagcaggaccagaggggaaggggctgctgcttgcatcagcccacaccatgctgaccccgccgttgctcctgctgctgcccctgctctcagctctggtcgcggcggctatcgacgcccctaagacttgcagccccaagcagtttgcctgcagagatcaaataacctgtatctcaaagggctggcggtgcgacggtgagagggactgcccagacggatctgacgaggcccctgagatttgtccacagagtaaggcccagcgatgccagccaaacgagcataactgcctgggtactgagctgtgtgttcccatgtcccgcctctgcaatggggtccaggactgcatggacggctcagatgaggggccccactgccgagagctccaaggcaactgctctcgcctgggctgccagcaccattgtgtccccacactcgatgggcccacctgctactgcaacagcagctttcagcttcaggcagatggcaagacctgcaaagattttgatgagtgctcagtgtacggcacctgcagccagctatgcaccaacacagacggctccttcatatgtggctgtgttgaaggatacctcctgcagccggataaccgctcctgcaaggccaagaacgagccagtagaccggccccctgtgctgttgatagccaactcccagaacatcttggccacgtacctgagtggggcccaggtgtctaccatcacacctacgagcacgcggcagaccacagccatggacttcagctatgccaacgagaccgtatgctgggtgcatgttggggacagtgctgctcagacgcagctcaagtgtgcccgcatgcctggcctaaagggcttcgtggatgagcacaccatcaacatctccctcagtctgcaccacgtggaacagatggccatcgactggctgacaggcaacttctactttgtggatgacatcgatgataggatctttgtctgcaacagaaatggggacacatgtgtcacattgctagacctggaactctacaaccccaagggcattgccctggaccctgccatggggaaggtgtttttcactgactatgggcagatcccaaaggtggaacgctgtgacatggatgggcagaaccgcaccaagctcgtcgacagcaagattgtgtttcctcatggcatcacgctggacctggtcagccgccttgtctactgggcagatgcctatctggactatattgaagtggtggactatgagggcaagggccgccagaccatcatccagggcatcctgattgagcacctgtacggcctgactgtgtttgagaattatctctatgccaccaactcggacaatgccaatgcccagcagaagacgagtgtgatccgtgtgaaccgctttaacagcaccgagtaccaggttgtcacccgggtggacaagggtggtgccctccacatctaccaccagaggcgtcagccccgagtgaggagccatgcctgtgaaaacgaccagtatgggaagccgggtggctgctctgacatctgcctgctggccaacagccacaaggcgcggacctgccgctgccgttccggcttcagcctgggcagtgacgggaagtcatgcaagaagccggagcatgagctgttcctcgtgtatggcaagggccggccaggcatcatccggggcatggatatgggggccaaggtcccggatgagcacatgatccccattgaaaacctcatgaacccccgagccctggacttccacgctgagaccggcttcatctactttgccgacaccaccagctacctcattggccgccagaagattgatggcactgagcgggagaccatcctgaaggacggcatccacaatgtggagggtgtggccgtggactggatgggagacaatctgtactggacggacgatgggcccaaaaagacaatcagcgtggccaggctggagaaagctgctcagacccgcaagactttaatcgagggcaaaatgacacaccccagggctattgtggtggatccactcaatgggtggatgtactggacagactgggaggaggaccccaaggacagtcggcgtgggcggctggagagggcgtggatggatggctcacaccgagacatctttgtcacctccaagacagtgctttggcccaatgggctaagcctggacatcccggctgggcgcctctactgggtggatgccttctacgaccgcatcgagacgatactgctcaatggcacagaccggaagattgtgtatgaaggtcctgagctgaaccacgcctttggcctgtgtcaccatggcaactacctcttctggactgagtatcggagtggcagtgtctaccgcttggaacggggtgtaggaggcgcaccccccactgtgacccttctgcgcagtgagcggccccccatctttgagatccgaatgtatgatgcccagcagcagcaagttggcaccaacaaatgccgggtgaacaatggcggctgcagcagcctgtgcttggccacccctgggagccgccagtgcgcctgtgctgaggaccaggtgttggacgcagacggcgtcacttgcttggcgaacccatcctacgtgcctccaccccagtgccagccaggcgagtttgcctgtgccaacagccgctgcatccaggagcgctggaagtgtgacggagacaacgattgcctggacaacagtgatgaggccccagccctctgccatcagcacacctgcccctcggaccgattcaagtgcgagaacaaccggtgcatccccaaccgctggctctgcgacggggacaatgactgtgggaacagtgaagatgagtccaatgccacttgttcagcccgcacctgcccccccaaccagttctcctgtgccagtggccgctgcatccccatctcctggacgtgtgatctggatgacgactgtggggaccgctctgatgagtctgcttcgtgtgcctatcccacctgcttccccctgactcagtttacctgcaacaatggcagatgtatcaacatcaactggagatgcgacaatgacaatgactgtggggacaacagtgacgaagccggctgcagccactcctgttctagcacccagttcaagtgcaacagcgggcgttgcatccccgagcactggacctgcgatggggacaatgactgcggagactacagtgatgagacacacgccaactgcaccaaccaggccacgaggccccctggtggctgccacactgatgagttccagtgccggctggatggactatgcatccccctgcggtggcgctgcgatggggacactgactgcatggactccagcgatgagaagagctgtgagggagtgacccacgtctgcgatcccagtgtcaagtttggctgcaaggactcagctcggtgcatcagcaaagcgtgggtgtgtgatggcgacaatgactgtgaggataactcggacgaggagaactgcgagtccctggcctgcaggccaccctcgcacccttgtgccaacaacacctcagtctgcctgccccctgacaagctgtgtgatggcaacgacgactgtggcgacggctcagatgagggcgagctctgcgaccagtgctctctgaataacggtggctgcagccacaactgctcagtggcacctggcgaaggcattgtgtgttcctgccctctgggcatggagctggggcccgacaaccacacctgccagatccagagctactgtgccaagcatctcaaatgcagccaaaagtgcgaccagaacaagttcagcgtgaagtgctcctgctacgagggctgggtcctggaacctgacggcgagagctgccgcagcctggaccccttcaagccgttcatcattttctccaaccgccatgaaatccggcgcatcgatcttcacaaaggagactacagcgtcctggtgcccggcctgcgcaacaccatcgccctggacttccacctcagccagagcgccctctactggaccgacgtggtggaggacaagatctaccgcgggaagctgctggacaacggagccctgactagtttcgaggtggtgattcagtatggcctggccacacccgagggcctggctgtagactggattgcaggcaacatctactgggtggagagtaacctggatcagatcgaggtggccaagctggatgggaccctccggaccaccctgctggccggtgacattgagcacccaagggcaatcgcactggatccccgggatgggatcctgttttggacagactgggatgccagcctgccccgcattgaggcagcctccatgagtggggctgggcgccgcaccgtgcaccgggagaccggctctgggggctggcccaacgggctcaccgtggactacctggagaagcgcatcctttggattgacgccaggtcagatgccatttactcagcccgttacgacggctctggccacatggaggtgcttcggggacacgagttcctgtcgcacccgtttgcagtgacgctgtacgggggggaggtctactggactgactggcgaacaaacacactggctaaggccaacaagtggaccggccacaatgtcaccgtggtacagaggaccaacacccagccctttgacctgcaggtgtaccacccctcccgccagcccatggctcccaatccctgtgaggccaatgggggccagggcccctgctcccacctgtgtctcatcaactacaaccggaccgtgtcctgcgcctgcccccacctcatgaagctccacaaggacaacaccacctgctatgagtttaagaagttcctgctgtacgcacgtcagatggagatccgaggtgtggacctggatgctccctactacaactacatcatctccttcacggtgcccgacatcgacaacgtcacagtgctagactacgatgcccgcgagcagcgtgtgtactggtctgacgtgcggacacaggccatcaagcgggccttcatcaacggcacaggcgtggagacagtcgtctctgcagacttgccaaatgcccacgggctggctgtggactgggtctcccgaaacctgttctggacaagctatgacaccaataagaagcagatcaatgtggcccggctggatggctccttcaagaacgcagtggtgcagggcctggagcagccccatggccttgtcgtccaccctctgcgtgggaagctctactggaccgatggtgacaacatcagcatggccaacatggatggcagcaatcgcaccctgctcttcagtggccagaagggccccgtgggcctggctattgacttccctgaaagcaaactctactggatcagctccgggaaccataccatcaaccgctgcaacctggatgggagtgggctggaggtcatcgatgccatgcggagccagctgggcaaggccaccgccctggccatcatgggggacaagctgtggtgggctgatcaggtgtcggaaaagatgggcacatgcagcaaggctgacggctcgggctccgtggtccttcggaacagcaccaccctggtgatgcacatgaaggtctatgacgagagcatccagctggaccataagggcaccaacccctgcagtgtcaacaacggtgactgctcccagctctgcctgcccacgtcagagacgacccgctcctgcatgtgcacagccggctatagcctccggagtggccagcaggcctgcgagggcgtaggttcctttctcctgtactctgtgcatgagggaatcaggggaattcccctggatcccaatgacaagtcagatgccctggtcccagtgtccgggacctcgctggctgtcggcatcgacttccacgctgaaaatgacaccatctactgggtggacatgggcctgagcacgatcagccgggccaagcgggaccagacgtggcgtgaagacgtggtgaccaatggcattggccgtgtggagggcattgcagtggactggatcgcaggcaacatctactggacagaccagggctttgatgtcatcgaggtcgcccggctcaatggctccttccgctacgtggtgatctcccagggtctagacaagccccgggccatcaccgtccacccggagaaagggtacttgttctggactgagtggggtcagtatccgcgtattgagcggtctcggctagatggcacggagcgtgtggtgctggtcaacgtcagcatcagctggcccaacggcatctcagtggactaccaggatgggaagctgtactggtgcgatgcacggacagacaagattgaacggatcgacctggagacaggtgagaaccgcgaggtggttctgtccagcaacaacatggacatgttttcagtgtctgtgtttgaggatttcatctactggagtgacaggactcatgccaacggctctatcaagcgcgggagcaaagacaatgccacagactccgtgcccctgcgaaccggcatcggcgtccagcttaaagacatcaaagtcttcaaccgggaccggcagaaaggcaccaacgtgtgcgcggtggccaatggcgggtgccagcagctgtgcctgtaccggggccgtgggcagcgggcctgcgcctgtgcccacgggatgctggctgaagacggagcatcgtgccgcgagtatgccggctacctgctctactcagagcgcaccattctcaagagtatccacctgtcggatgagcgcaacctcaatgcgcccgtgcagcccttcgaggaccctgagcacatgaagaacgtcatcgccctggcctttgactaccgggcaggcacctctccgggcacccccaatcgcatcttcttcagcgacatccactttgggaacatccaacagatcaacgacgatggctccaggaggatcaccattgtggaaaacgtgggctccgtggaaggcctggcctatcaccgtggctgggacactctctattggacaagctacacgacatccaccatcacgcgccacacagtggaccagacccgcccaggggccttcgagcgtgagaccgtcatcactatgtctggagatgaccacccacgggccttcgttttggacgagtgccagaacctcatgttctggaccaactggaatgagcagcatcccagcatcatgcgggcggcgctctcgggagccaatgtcctgacccttatcgagaaggacatccgtacccccaatggcctggccatcgaccaccgtgccgagaagctctacttctctgacgccaccctggacaagatcgagcggtgcgagtatgacggctcccaccgctatgtgatcctaaagtcagagcctgtccaccccttcgggctggccgtgtatggggagcacattttctggactgactgggtgcggcgggcagtgcagcgggccaacaagcacgtgggcagcaacatgaagctgctgcgcgtggacatcccccagcagcccatgggcatcatcgccgtggccaacgacaccaacagctgtgaactctctccatgccgaatcaacaacggtggctgccaggacctgtgtctgctcactcaccagggccatgtcaactgctcatgccgagggggccgaatcctccaggatgacctcacctgccgagcggtgaattcctcttgccgagcacaagatgagtttgagtgtgccaatggcgagtgcatcaacttcagcctgacctgcgacggcgtcccccactgcaaggacaagtccgatgagaagccatcctactgcaactcccgccgctgcaagaagactttccggcagtgcagcaatgggcgctgtgtgtccaacatgctgtggtgcaacggggccgacgactgtggggatggctctgacgagatcccttgcaacaagacagcctgtggtgtgggcgagttccgctgccgggacgggacctgcatcgggaactccagccgctgcaaccagtttgtggattgtgaggacgcctcagatgagatgaactgcagtgccaccgactgcagcagctacttccgcctgggcgtgaagggcgtgctcttccagccctgcgagcggacctcactctgctacgcacccagctgggtgtgtgatggcgccaatgactgtggggactacagtgatgagcgcgactgcccaggtgtgaaacgccccagatgccctctgaattacttcgcctgccctagtgggcgctgcatccccatgagctggacgtgtgacaaagaggatgactgtgaacatggcgaggacgagacccactgcaacaagttctgctcagaggcccagtttgagtgccagaaccatcgctgcatctccaagcagtggctgtgtgacggcagcgatgactgtggggatggctcagacgaggctgctcactgtgaaggcaagacgtgcggcccctcctccttctcctgccctggcacccacgtgtgcgtccccgagcgctggctctgtgacggtgacaaagactgtgctgatggtgcagacgagagcatcgcagctggttgcttgtacaacagcacttgtgacgaccgtgagttcatgtgccagaaccgccagtgcatccccaagcacttcgtgtgtgaccacgaccgtgactgtgcagatggctctgatgagtcccccgagtgtgagtacccgacctgcggccccagtgagttccgctgtgccaatgggcgctgtctgagctcccgccagtgggagtgtgatggcgagaatgactgccacgaccagagtgacgaggctcccaagaacccacactgcaccagcccagagcacaagtgcaatgcctcgtcacagttcctgtgcagcagtgggcgctgtgtggctgaggcactgctctgcaacggccaggatgactgtggcgacagctcggacgagcgtggctgccacatcaatgagtgtctcagccgcaagctcagtggctgcagccaggactgtgaggacctcaagatcggcttcaagtgccgctgtcgccctggcttccggctgaaggatgacggccggacgtgtgctgatgtggacgagtgcagcaccaccttcccctgcagccagcgctgcatcaacacccatggcagctataagtgtctgtgtgtggagggctatgcaccccgcggcggcgacccccacagctgcaaggctgtgactgacgaggaaccgtttctgatcttcgccaaccggtactacctgcgcaagctcaacctggacgggtccaactacacgttacttaagcagggcctgaacaacgccgttgccttggattttgactaccgagagcagatgatctactggacagatgtgaccacccagggcagcatgatccgaaggatgcaccttaacgggagcaatgtgcaggtcctacaccgtacaggcctcagcaaccccgatgggctggctgtggactgggtgggtggcaacctgtactggtgcgacaaaggccgggacaccatcgaggtgtccaagctcaatggggcctatcggacggtgctggtcagctctggcctccgtgagcccagggctctggtggtggatgtgcagaatgggtacctgtactggacagactggggtgaccattcactgatcggccgcatcggcatggatgggtccagccgcagcgtcatcgtggacaccaagatcacatggcccaatggcctgacgctggactatgtcactgagcgcatctactgggccgacgcccgcgaggactacattgaatttgccagcctggatggctccaatcgccacgttgtgctgagccaggacatcccgcacatctttgcactgaccctgtttgaggactacgtctactggaccgactgggaaacaaagtccattaaccgagcccacaagaccacgggcaccaacaaaacgctcctcatcagcacgctgcaccggcccatggacctgcatgtcttccatgccctgcgccagccagacgtgcccaatcacccctgcaaggtcaacaatggtggctgcagcaacctgtgcctgctgtcccccgggggagggcacaaatgtgcctgccccaccaacttctacctgggcagcgatgggcgcacctgtgtgtccaactgcacggctagccagtttgtatgcaagaacgacaagtgcatccccttctggtggaagtgtgacaccgaggacgactgcggggaccactcagacgagcccccggactgccctgagttcaagtgccggcccggacagttccagtgctccacaggtatctgcacaaaccctgccttcatctgcgatggcgacaatgactgccaggacaacagtgacgaggccaactgtgacatccacgtctgcttgcccagtcagttcaaatgcaccaacaccaaccgctgtattcccggcatcttccgctgcaatgggcaggacaactgcggagatggggaggatgagagggactgccccgaggtgacctgcgcccccaaccagttccagtgctccattaccaaacggtgcatcccccgggtctgggtctgcgaccgggacaatgactgtgtggatggcagtgatgagcccgccaactgcacccagatgacctgtggtgtggacgagttccgctgcaaggattcgggccgctgcatcccagcgcgttggaagtgtgacggagaggatgactgtggggatggctcggatgagcccaaggaagagtgtgatgaacgcacctgtgagccataccagttccgctgcaagaacaaccgctgcgtgcccggccgctggcagtgcgactacgacaacgattgcggtgacaactccgatgaagagagctgcacccctcggccctgctccgagagtgagttctcctgtgccaacggccgctgcatcgcggggcgctggaaatgcgatggagaccacgactgcgcggacggctcggacgagaaagactgcaccccccgctgtgacatggaccagttccagtgcaagagcggccactgcatccccctgcgctggcgctgtgacgcagacgccgactgcatggacggcagcgacgaggaggcctgcggcactggcgtgcggacctgccccctggacgagttccagtgcaacaacaccttgtgcaagccgctggcctggaagtgcgatggcgaggatgactgtggggacaactcagatgagaaccccgaggagtgtgcccggttcgtgtgccctcccaaccggcccttccgttgcaagaatgaccgcgtctgtctgtggatcgggcgccaatgcgatggcacggacaactgtggggatgggactgatgaagaggactgtgagccccccacagcccacaccacccactgcaaagacaagaaggagtttctgtgccggaaccagcgctgcctctcctcctccctgcgctgcaacatgttcgatgactgcggggacggctctgacgaggaggactgcagcatcgaccccaagctgaccagctgcgccaccaatgccagcatctgtggggacgaggcacgctgcgtgcgcaccgagaaagcggcctactgtgcctgccgctcgggcttccacaccgtgcccggccagcccggatgccaagacatcaacgagtgcctgcgcttcggcacctgctcccagctctgcaacaacaccaagggcggccacctctgcagctgcgctcggaacttcatgaagacgcacaacacctgcaaggccgaaggctctgagtaccaggtcctgtacatcgctgatgacaatgagatccgcagcctgttccccggccacccccattcggcttacgagcaggcattccagggtgacgagagtgtccgcattgatgctatggatgtccatgtcaaggctggccgtgtctattggaccaactggcacacgggcaccatctcctaccgcagcctgccacctgctgcgcctcctaccacttccaaccgccaccggcgacagattgaccggggtgtcacccacctcaacatttcagggctgaagatgcccagaggcatcgccatcgactgggtggccggaaacgtgtactggaccgactcgggccgagatgtgattgaggtggcgcagatgaagggcgagaaccgcaagacgctcatctcgggcatgattgacgagccccacgccattgtggtggacccactgagggggaccatgtactggtcagactggggcaaccaccccaagattgagacggcagcgatggatgggacgcttcgggagacactggtgcaggacaacattcagtggcccacaggcctggccgtggattatcacaatgagcggctgtactgggcagacgccaagctttcagtcatcggcagcatccggctcaatggcacggaccccattgtggctgctgacagcaaacgaggcctaagtcaccccttcagcatcgacgtctttgaggattacatctatggtgtcacctacatcaataatcgtgtcttcaagatccataagtttggccacagccccttggtcaacctgacagggggcctgagccacgcctctgacgtggtcctttaccatcagcacaagcagcccgaagtgaccaacccatgtgaccgcaagaaatgcgagtggctctgcctgctgagccccagtgggcctgtctgcacctgtcccaatgggaagcggctggacaacggcacatgcgtgcctgtgccctctccaacgccccccccagatgctccccggcctggaacctgtaacctgcagtgcttcaacggtggcagctgtttcctcaatgcacggaggcagcccaagtgccgctgccaaccccgctacacgggtgacaagtgtgaactggaccagtgctgggagcactgtcgcaatgggggcacctgtgctgcctccccctctggcatgcccacgtgccggtgccccacgggcttcacgggccccaaatgcacccagcaggtgtgtgcgggctactgtgccaacaacagcacctgcactgtcaaccagggcaaccagccccagtgccgatgcctacccggcttcctgggcgaccgctgccagtaccggcagtgctctggctactgtgagaactttggcacatgccagatggctgctgatggctcccgacaatgccgctgcactgcctactttgagggatcgaggtgtgaggtgaacaagtgcagccgctgtctcgaaggggcctgtgtggtcaacaagcagagtggggatgtcacctgcaactgcacggatggccgggtggcccccagctgtctgacctgcgtcggccactgcagcaatggcggctcctgtaccatgaacagcaaaatgatgcctgagtgccagtgcccaccccacatgacagggccccggtgtgaggagcacgtcttcagccagcagcagccaggacatatagcctccatcctaatccctctgctgttgctgctgctgctggttctggtggccggagtggtattctggtataagcggcgagtccaaggggctaagggcttccagcaccaacggatgaccaacggggccatgaacgtggagattggaaaccccacctacaagatgtacgaaggcggagagcctgatgatgtgggaggcctactggacgctgactttgccctggaccctgacaagcccaccaacttcaccaaccccgtgtatgccacactctacatggggggccatggcagtcgccactccctggccagcacggacgagaagcgagaactcctgggccggggccctgaggacgagataggggaccccttggcatagggccctgccccgtcggactgcccccagaaagcctcctgccccctgccggtgaagtccttcagtgagcccctccccagccagcccttccctggccccgccggatgtataaatgtaaaaatgaaggaattacattttatatgtgagcgagcaagccggcaagcgagcacagtattatttctccatcccctccctgcctgctccttggcacccccatgctgccttcagggagacaggcagggagggcttggggctgcacctcctaccctcccaccagaacgcaccccactgggagagctggtggtgcagccttcccctccctgtataagacactttgccaaggctctcccctctcgccccatccctgcttgcccgctcccacagcttcctgagggctaattctgggaagggagagttctttgctgcccctgtctggaagacgtggctctgggtgaggtaggcgggaaaggatggagtgttttagttcttgggggaggccaccccaaaccccagccccaactccaggggcacctatgagatggccatgctcaacccccctcccagacaggccctccctgtctccagggcccccaccgaggttcccagggctggagacttcctctggtaaacattcctccagcctcccctcccctggggacgccaaggaggtgggccacacccaggaagggaaagcgggcagccccgttttggggacgtgaacgttttaataatttttgctgaattctttacaactaaataacacagatattcttataaataaaattgtaaaaaaaaaaa

SEQ ID NO: 32 - protein, 4544 aa, Homo sapiens
MLTPPLLLLLPLLSALVAAAIDAPKTCSPKQFACRDQITCISKGWRCDGERDCPDGSDEAPEICPQSKAQRCQPNEHNCLGTELCVPMSRLCNGVQDCMDGSDEGPHCRELQGNCSRLGCQHHCVPTLDGPTCYCNSSFQLQADGKTCKDFDECSVYGTCSQLCTNTDGSFICGCVEGYLLQPDNRSCKAKNEPVDRPPVLLIANSQNILATYLSGAQVSTITPTSTRQTTAMDFSYANETVCWVHVGDSAAQTQLKCARMPGLKGFVDEHTINISLSLHHVEQMAIDWLTGNFYFVDDIDDRIFVCNRNGDTCVTLLDLELYNPKGIALDPAMGKVFFTDYGQIPKVERCDMDGQNRTKLVDSKIVFPHGITLDLVSRLVYWADAYLDYIEVVDYEGKGRQTIIQGILIEHLYGLTVFENYLYATNSDNANAQQKTSVIRVNRFNSTEYQVVTRVDKGGALHIYHQRRQPRVRSHACENDQYGKPGGCSDICLLANSHKARTCRCRSGFSLGSDGKSCKKPEHELFLVYGKGRPGIIRGMDMGAKVPDEHMIPIENLMNPRALDFHAETGFIYFADTTSYLIGRQKIDGTERETILKDGIHNVEGVAVDWMGDNLYWTDDGPKKTISVARLEKAAQTRKTLIEGKMTHPRAIVVDPLNGWMYWTDWEEDPKDSRRGRLERAWMDGSHRDIFVTSKTVLWPNGLSLDIPAGRLYWVDAFYDRIETILLNGTDRKIVYEGPELNHAFGLCHHGNYLFWTEYRSGSVYRLERGVGGAPPTVTLLRSERPPIFEIRMYDAQQQQVGTNKCRVNNGGCSSLCLATPGSRQCACAEDQVLDADGVTCLANPSYVPPPQCQPGEFACANSRCIQERWKCDGDNDCLDNSDEAPALCHQHTCPSDRFKCENNRCIPNRWLCDGDNDCGNSEDESNATCSARTCPPNQFSCASGRCIPISWTCDLDDDCGDRSDESASCAYPTCFPLTQFTCNNGRCININWRCDNDNDCGDNSDEAGCSHSCSSTQFKCNSGRCIPEHWTCDGDNDCGDYSDETHANCTNQATRPPGGCHTDEFQCRLDGLCIPLRWRCDGDTDCMDSSDEKSCEGVTHVCDPSVKFGCKDSARCISKAWVCDGDNDCEDNSDEENCESLACRPPSHPCANNTSVCLPPDKLCDGNDDCGDGSDEGELCDQCSLNNGGCSHNCSVAPGEGIVCSCPLGMELGPDNHTCQIQSYCAKHLKCSQKCDQNKFSVKCSCYEGWVLEPDGESCRSLDPFKPFIIFSNRHEIRRIDLHKGDYSVLVPGLRNTIALDFHLSQSALYWTDVVEDKIYRGKLLDNGALTSFEVVIQYGLATPEGLAVDWIAGNIYWVESNLDQIEVAKLDGTLRTTLLAGDIEHPRAIALDPRDGILFWTDWDASLPRIEAASMSGAGRRTVHRETGSGGWPNGLTVDYLEKRILWIDARSDAIYSARYDGSGHMEVLRGHEFLSHPFAVTLYGGEVYWTDWRTNTLAKANKWTGHNVTVVQRTNTQPFDLQVYHPSRQPMAPNPCEANGGQGPCSHLCLINYNRTVSCACPHLMKLHKDNTTCYEFKKFLLYARQMEIRGVDLDAPYYNYIISFTVPDIDNVTVLDYDAREQRVYWSDVRTQAIKRAFINGTGVETVVSADLPNAHGLAVDWVSRNLFWTSYDTNKKQINVARLDGSFKNAVVQGLEQPHGLVVHPLRGKLYWTDGDNISMANMDGSNRTLLFSGQKGPVGLAIDFPESKLYWISSGNHTINRCNLDGSGLEVIDAMRSQLGKATALAIMGDKLWWADQVSEKMGTCSKADGSGSVVLRNSTTLVMHMKVYDESIQLDHKGTNPCSVNNGDCSQLCLPTSETTRSCMCTAGYSLRSGQQACEGVGSFLLYSVHEGIRGIPLDPNDKSDALVPVSGTSLAVGIDFHAENDTIYWVDMGLSTISRAKRDQTWREDVVTNGIGRVEGIAVDWIAGNIYWTDQGFDVIEVARLNGSFRYVVISQGLDKPRAITVHPEKGYLFWTEWGQYPRIERSRLDGTERVVLVNVSISWPNGISVDYQDGKLYWCDARTDKIERIDLETGENREVVLSSNNMDMFSVSVFEDFIYWSDRTHANGSIKRGSKDNATDSVPLRTGIGVQLKDIKVFNRDRQKGTNVCAVANGGCQQLCLYRGRGQRACACAHGMLAEDGASCREYAGYLLYSERTILKSIHLSDERNLNAPVQPFEDPEHMKNVIALAFDYRAGTSPGTPNRIFFSDIHFGNIQQINDDGSRRITIVENVGSVEGLAYHRGWDTLYWTSYTTSTITRHTVDQTRPGAFERETVITMSGDDHPRAFVLDECQNLMFWTNWNEQHPSIMRAALSGANVLTLIEKDIRTPNGLAIDHRAEKLYFSDATLDKIERCEYDGSHRYVILKSEPVHPFGLAVYGEHIFWTDWVRRAVQRANKHVGSNMKLLRVDIPQQPMGIIAVANDTNSCELSPCRINNGGCQDLCLLTHQGHVNCSCRGGRILQDDLTCRAVNSSCRAQDEFECANGECINFSLTCDGVPHCKDKSDEKPSYCNSRRCKKTFRQCSNGRCVSNMLWCNGADDCGDGSDEIPCNKTACGVGEFRCRDGTCIGNSSRCNQFVDCEDASDEMNCSATDCSSYFRLGVKGVLFQPCERTSLCYAPSWVCDGANDCGDYSDERDCPGVKRPRCPLNYFACPSGRCIPMSWTCDKEDDCEHGEDETHCNKFCSEAQFECQNHRCISKQWLCDGSDDCGDGSDEAAHCEGKTCGPSSFSCPGTHVCVPERWLCDGDKDCADGADESIAAGCLYNSTCDDREFMCQNRQCIPKHFVCDHDRDCADGSDESPECEYPTCGPSEFRCANGRCLSSRQWECDGENDCHDQSDEAPKNPHCTSPEHKCNASSQFLCSSGRCVAEALLCNGQDDCGDSSDERGCHINECLSRKLSGCSQDCEDLKIGFKCRCRPGFRLKDDGRTCADVDECSTTFPCSQRCINTHGSYKCLCVEGYAPRGGDPHSCKAVTDEEPFLIFANRYYLRKLNLDGSNYTLLKQGLNNAVALDFDYREQMIYWTDVTTQGSMIRRMHLNGSNVQVLHRTGLSNPDGLAVDWVGGNLYWCDKGRDTIEVSKLNGAYRTVLVSSGLREPRALVVDVQNGYLYWTDWGDHSLIGRIGMDGSSRSVIVDTKITWPNGLTLDYVTERIYWADAREDYIEFASLDGSNRHVVLSQDIPHIFALTLFEDYVYWTDWETKSINRAHKTTGTNKTLLISTLHRPMDLHVFHALRQPDVPNHPCKVNNGGCSNLCLLSPGGGHKCACPTNFYLGSDGRTCVSNCTASQFVCKNDKCIPFWWKCDTEDDCGDHSDEPPDCPEFKCRPGQFQCSTGICTNPAFICDGDNDCQDNSDEANCDIHVCLPSQFKCTNTNRCIPGIFRCNGQDNCGDGEDERDCPEVTCAPNQFQCSITKRCIPRVWVCDRDNDCVDGSDEPANCTQMTCGVDEFRCKDSGRCIPARWKCDGEDDCGDGSDEPKEECDERTCEPYQFRCKNNRCVPGRWQCDYDNDCGDNSDEESCTPRPCSESEFSCANGRCIAGRWKCDGDHDCADGSDEKDCTPRCDMDQFQCKSGHCIPLRWRCDADADCMDGSDEEACGTGVRTCPLDEFQCNNTLCKPLAWKCDGEDDCGDNSDENPEECARFVCPPNRPFRCKNDRVCLWIGRQCDGTDNCGDGTDEEDCEPPTAHTTHCKDKKEFLCRNQRCLSSSLRCNMFDDCGDGSDEEDCSIDPKLTSCATNASICGDEARCVRTEKAAYCACRSGFHTVPGQPGCQDINECLRFGTCSQLCNNTKGGHLCSCARNFMKTHNTCKAEGSEYQVLYIADDNEIRSLFPGHPHSAYEQAFQGDESVRIDAMDVHVKAGRVYWTNWHTGTISYRSLPPAAPPTTSNRHRRQIDRGVTHLNISGLKMPRGIAIDWVAGNVYWTDSGRDVIEVAQMKGENRKTLISGMIDEPHAIVVDPLRGTMYWSDWGNHPKIETAAMDGTLRETLVQDNIQWPTGLAVDYHNERLYWADAKLSVIGSIRLNGTDPIVAADSKRGLSHPFSIDVFEDYIYGVTYINNRVFKIHKFGHSPLVNLTGGLSHASDVVLYHQHKQPEVTNPCDRKKCEWLCLLSPSGPVCTCPNGKRLDNGTCVPVPSPTPPPDAPRPGTCNLQCFNGGSCFLNARRQPKCRCQPRYTGDKCELDQCWEHCRNGGTCAASPSGMPTCRCPTGFTGPKCTQQVCAGYCANNSTCTVNQGNQPQCRCLPGFLGDRCQYRQCSGYCENFGTCQMAADGSRQCRCTAYFEGSRCEVNKCSRCLEGACVVNKQSGDVTCNCTDGRVAPSCLTCVGHCSNGGSCTMNSKMMPECQCPPHMTGPRCEEHVFSQQQPGHIASILIPLLLLLLLVLVAGVVFWYKRRVQGAKGFQHQRMTNGAMNVEIGNPTYKMYEGGEPDDVGGLLDADFALDPDKPTNFTNPVYATLYMGGHGSRHSLASTDEKRELLGRGPEDEIGDPLA

SEQ ID NO: 33 - DNA, 538 bp, Artificial sequence
  Promoter sequence (CMV)
gcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtct

SEQ ID NO: 34 - DNA, 3088 bp, Homo sapiens
ccccttttccagaatcacttgcactgtcttgttcttgaatgagaaaggaagaaaagagcctcccattactcagacccgtgtaaacattattccccccaggagaaaatggtgttattcaaatgaatcataataaaatagcctctaaacagtttctaagcgggagcctccgtggaactcagcgctccgctcctcccagttcctaagaggtcccgggattcttgagctgtgcccagctgacgagcttttgaagatggcacaataaccgtccagtgatgcctgaccatgacagcacagccctcttaagccggcaaaccaagaggagaagagttgacattggagtgaaaaggacggtagggacagcatctgcattttttgctaaggcaagagcaacgttttttagtgccatgaatccccaaggttctgagcaggatgttgagtattcagtggtgcagcatgcagatggggaaaagtcaaatgtactccgcaagctgctgaagagggcgaactcgtatgaagatgccatgatgccttttccaggagcaaccataatttcccagctgttgaaaaataacatgaacaaaaatggtggcacggagcccagtttccaagccagcggtctctctagtacaggctccgaagtacatcaggaggatatatgcagcaactcttcaagagacagccccccagagtgtctttccccttttggcaggcctactatgagccagtttgatatggatcgcttatgtgatgagcacctgagagcaaagcgcgcccgggttgagaatataattcggggtatgagccattcccccagtgtggcattaaggggcaatgaaaatgaaagagagatggccccgcagtctgtgagtccccgagaaagttacagagaaaacaaacgcaagcaaaagcttccccagcagcagcaacagagtttccagcagctggtttcagcccgaaaagaacagaagcgagaggagcgccgacagctgaaacagcagctggaggacatgcagaaacagctgcgccagctgcaggaaaagttctaccaaatctatgacagcactgattcggaaaatgatgaagatggtaacctgtctgaagacagcatgcgctcggagatcctggatgccagggcccaggactctgtcggaaggtcagataatgagatgtgcgagctagacccaggacagtttattgaccgagctcgagccctgatcagagagcaggaaatggctgaaaacaagccgaagcgagaaggcaacaacaaagaaagagaccatgggccaaactccttacaaccggaaggcaaacatttggctgagaccttgaaacaggaactgaacactgccatgtcgcaagttgtggacactgtggtcaaagtcttttcggccaagccctcccgccaggttcctcaggtcttcccacctctccagatcccccaggccagatttgcagtcaatggggaaaaccacaatttccacaccgccaaccagcgcctgcagtgctttggcgacgtcatcattccgaaccccctggacacctttggcaatgtgcagatggccagttccactgaccagacagaagcactgcccctggttgtccgcaaaaactcctctgaccagtctgcctccggccctgccgctggcggccaccaccagcccctgcaccagtcgcctctctctgccaccacgggcttcaccacgtccaccttccgccaccccttcccccttcccttgatggcctatccatttcagagcccattaggtgctccctccggctccttctctggaaaagacagagcctctcctgaatccttagacttaactagggataccacgagtctgaggaccaagatgtcatctcaccacctgagccaccacccttgttcaccagcacacccgcccagcaccgccgaagggctctccttgtcgctcataaagtccgagtgcggcgatcttcaagatatgtctgaaatatcaccttattcgggaagtgcaatgcaggaaggattgtcacccaatcacttgaaaaaagcaaagctcatgtttttttatacccgttatcccagctccaatatgctgaagacctacttctccgacgtaaagttcaacagatgcattacctctcagctcatcaagtggtttagcaatttccgtgagttttactacattcagatggagaagtacgcacgtcaagccatcaacgatggggtcaccagtactgaagagctgtctataaccagagactgtgagctgtacagggctctgaacatgcactacaataaagcaaatgactttgaggttccagagagattcctggaagttgctcagatcacattacgggagtttttcaatgccattatcgcaggcaaagatgttgatccttcctggaagaaggccatatacaaggtcatctgcaagctggatagtgaagtccctgagattttcaaatccccgaactgcctacaagagctgcttcatgagtagaaatttcaacaactctttttgaatgtatgaagagtagcagtcccctttggatgtccaagttatatgtgtctagattttgatttcatatatatgtgtatgggaggcatggatatgttatgaaatcagctggtaattcctcctcatcacgtttctctcattttcttttgttttccattgcaaggggatggttgttttctttctgcctttagtttgcttttgcccaaggcccttaacatttggacacttaaaatagggttaattttcagggaaaaagaatgttggcgtgtgtaaagtctctattagcaatgaagggaatttgttaacgatgcatccacttgattgatgacttattgcaaatggcggttggctgaggaaaacccatgacacagcacaactctacagacagtgatgtgtctcttgtttctactgctaagaaggtctgaaaatttaatgaaaccacttcatacatttaagtattttgtttggtttgaactcaatcagtagcttttccttacatgtttaaaaataattccaatgacagatgagcagctcacttttccaaagtaccccaaaaggccaaattaaaaaaaaaaaaaaaaaaa

SEQ ID NO: 35 - protein, 737 aa, Homo sapiens
MPDHDSTALLSRQTKRRRVDIGVKRTVGTASAFFAKARATFFSAMNPQGSEQDVEYSVVQHADGEKSNVLRKLLKRANSYEDAMMPFPGATIISQLLKNNMNKNGGTEPSFQASGLSSTGSEVHQEDICSNSSRDSPPECLSPFGRPTMSQFDMDRLCDEHLRAKRARVENIIRGMSHSPSVALRGNENEREMAPQSVSPRESYRENKRKQKLPQQQQQSFQQLVSARKEQKREERRQLKQQLEDMQKQLRQLQEKFYQIYDSTDSENDEDGNLSEDSMRSEILDARAQDSVGRSDNEMCELDPGQFIDRARALIREQEMAENKPKREGNNKERDHGPNSLQPEGKHLAETLKQELNTAMSQVVDTVVKVFSAKPSRQVPQVFPPLQIPQARFAVNGENHNFHTANQRLQCFGDVIIPNPLDTFGNVQMASSTDQTEALPLVVRKNSSDQSASGPAAGGHHQPLHQSPLSATTGFTTSTFRHPFPLPLMAYPFQSPLGAPSGSFSGKDRASPESLDLTRDTTSLRTKMSSHHLSHHPCSPAHPPSTAEGLSLSLIKSECGDLQDMSEISPYSGSAMQEGLSPNHLKKAKLMFFYTRYPSSNMLKTYFSDVKFNRCITSQLIKWFSNFREFYYIQMEKYARQAINDGVTSTEELSITRDCELYRALNMHYNKANDFEVPERFLEVAQITLREFFNAIIAGKDVDPSWKKAIYKVICKLDSEVPEIFKSPNCLQELLHE